(19) United States
(12) Patent Application Publication
(10) Pub. No.: US 2024/0197756 A1
(43) Pub. Date: Jun. 20, 2024

(54) METHODS OF TREATMENT WITH NEUROACTIVE STEROIDS

(71) Applicant: Praxis Precision Medicines, Inc., Boston, MA (US)

(72) Inventors: Carlos LOYA, Cambridge, MA (US); Nicholas DEMARTINIS, Cambridge, MA (US); Gabriel BELFORT, Cambridge, MA (US)

(21) Appl. No.: 18/555,222

(22) PCT Filed: Apr. 26, 2022
(86) PCT No.: PCT/US2022/026289

§ 371 Date: Oct. 12, 2023

Related U.S. Application Data
(60) Provisional application No. 63/180,003, filed on Apr. 26, 2021.

(51) Int. Cl. A61K 31/58; A61K 31/445; A61P 25/14; A61P 25/24

(52) U.S. Cl. CPC A61K 31/58; A61K 31/445; A61P 25/14; A61P 25/24

(57) ABSTRACT

The disclosure relates to methods of treating depression, pain, a musculoskeletal disorder or a motor disorder such as essential tremor with Compound 1 or pharmaceutically acceptable salts thereof.

### CROSS-REFERENCE TO RELATED APPLICATION

This application claims the benefit of, and relies on the filing date of, U.S. provisional patent application No. 63/180,003, filed 26 Apr. 2021, the entire disclosure of which is incorporated herein by reference.

### FIELD OF THE DISCLOSURE

The present disclosure relates to methods of treatment using neurosteroids such as 3α-hydroxy-3β-methoxymethyl-21-(1′-imidazolyl)-5α-pregnan-20-one and salts or deuterated forms thereof.

### BACKGROUND OF THE DISCLOSURE

3α-Hydroxy-3β-methoxymethyl-21-(1′-imidazolyl)-5α-pregnan-20-one (Compound 1) is a synthetic neuroactive steroid. Its primary molecular target is the γ-aminobutyric acid type A (GABA_{A}) receptor, where it acts as a positive allosteric modulator (PAM) of channel function. The structural formula of Compound 1 appears below.

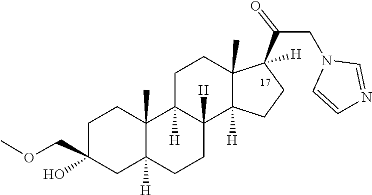

Neuroactive steroid GABA_{A} PAMs have demonstrated clinical efficacy in anesthesia, epilepsy, post-partum depression, and major depression.

### SUMMARY OF THE DISCLOSURE

The present disclosure, among other things, provides methods of treating depression by administering a therapeutically effective amount of a neurosteroid, such as Compound 1 or a pharmaceutically acceptable salt or an isotopically labeled form thereof (e.g., deuterated forms such as those described in PCT/US2021/018568 (herein incorporated by reference in its entirety), to a patient in need thereof. In all instances of the present disclosure, “Compound 1” refers to both a non-deuterated form of Compound 1 and a deuterated form of Compound 1 as described herein. In another aspect, the present disclosure provides methods of treating a mood or affective disorder selected from perimenopause, generalized anxiety disorder, panic disorder, social anxiety disorder, post-traumatic stress disorder, acute stress disorder, specific phobia, and selective mutism by administering a therapeutically effective amount of a neurosteroid such as Compound 1 or a pharmaceutically acceptable salt to a patient in need of thereof. In still other embodiments, the present disclosure provides methods of treating various movement disorders including various forms of tremors by administering a therapeutically effective amount of a neurosteroid, such as Compound 1 or a pharmaceutically acceptable salt thereof, to a patient in need thereof. In yet other embodiments, the present disclosure provides methods of treating various tremors by administering a therapeutically effective amount of a neurosteroid, such as Compound 1 or a pharmaceutically acceptable salt thereof, in combination with a T-type calcium channel blocker to a patient in need thereof. In yet other embodiments, the present disclosure provides methods of treating musculoskeletal disorders, including fibromyalgia, polymyalgia, chronic fatigue syndrome, systemic exertion intolerance disease, chronic pain, osteoarthritis, rheumatoid arthritis, psoriatic arthritis, gout, ankylosing spondylitis, osteoporosis, and similar conditions. In yet other embodiments, the present disclosure provides methods of treating pain disorders including chronic pain, peripheral neuropathy, diabetic peripheral neuropathy, acute pain, post-operative pain, low back pain, visceral pain, carpal tunnel syndrome, trigeminal neuralgia, trigeminal autonomic cephalgias, cluster headache, paroxysmal hemicrania, short-lasting unilateral neuralgiform headache attacks with conjunctival injection and tearing, short-lasting unilateral neuralgiform headache attacks with cranial autonomic symptoms, and similar conditions. In some embodiments, the present disclosure provides methods of treating acute stress disorder. In some embodiments, the present disclosure provides methods of treating post-traumatic stress disorder. In another aspect, the present disclosure provides methods of treating a substance abuse disorder by administering a therapeutically effective amount of a neurosteroid, such as Compound 1 or a pharmaceutically acceptable salt thereof, to a patient in need of thereof. In some embodiments, the method comprises orally administering a daily dose of about 5 mg to about 120 mg of Compound 1 or a pharmaceutically acceptable salt thereof to a patient in need thereof.

In some embodiments, the patient in need of a treatment of depression is a patient with major depressive disorder (MDD). In certain embodiments, the patient has moderate MDD. In certain embodiments, the patient has severe MDD. In certain embodiments, the patient in need of a treatment of depression is a patient with MDD and insomnia. In certain embodiments, the patient in need of a treatment of depression is a patient with anxious MDD and insomnia. In certain embodiments, the patient in need of treatment of depression is a patient with MDD with anxious distress.

In some embodiments, the patient in need of a treatment of a movement disorder, such as a tremor, is a patient with essential tremor. In certain embodiments, the patient has a tremor selected from the group consisting of cerebellar tremor or intention tremor, dystonic tremor, essential tremor, orthostatic tremor, Parkinsonian tremor, physiological tremor, psychogenic tremor, or rubral tremor. Tremor includes hereditary, degenerative, and idiopathic disorders such as Wilson's disease, Parkinson's disease, and essential tremor, respectively; metabolic diseases (e.g., thyroid disorders, parathyroid disorders, liver disease, and hypoglycemia); peripheral neuropathies (associated with Charcot-Marie-Tooth syndrome, Roussy Levy syndrome, diabetes mellitus, complex regional pain syndrome); toxins (nicotine, mercury, lead, cobalt, manganese, arsenic, toluene); drug-induced (narcoleptics, tricyclics, lithium, cocaine, alcohol, adrenaline, bronchodilators, theophylline, caffeine, steroids, valproate, amiodarone, thyroid hormones, vincristine); and psychogenic disorders. Clinical tremor can be classified into physiologic tremor, enhanced physiologic tremor, essential tremor syndromes (including classical essential tremor, primary orthostatic tremor, and task- and position-specific tremor), dystonic tremor, parkinsonian tremor, cerebellar tremor, Holmes' tremor (i.e., rubral tremor), palatal tremor, neuropathic tremor, toxic or drug-induced tremor, and psychogenic tremor.

In some embodiments, the patient in need of treatment has a musculoskeletal disorder such as fibromyalgia or related musculoskeletal disorders. Related musculoskeletal disorders include polymyalgia, chronic fatigue syndrome, systemic exertion intolerance disease, chronic pain, osteoarthritis, rheumatoid arthritis, psoriatic arthritis, gout, ankylosing spondylitis, osteoporosis, and similar conditions. In yet other embodiments, the present disclosure provides methods of treating pain disorders, including chronic pain, peripheral neuropathy, diabetic peripheral neuropathy, acute pain, post-operative pain, low back pain, visceral pain, carpal tunnel syndrome, trigeminal neuralgia, trigeminal autonomic cephalgias, cluster headache, paroxysmal hemicrania, short-lasting unilateral neuralgiform headache attacks with conjunctival injection and tearing, short-lasting unilateral neuralgiform headache attacks with cranial autonomic symptoms, and similar conditions.

In some embodiments, the present disclosure provides adjunctive treatment for major depression comprising administering an effective amount of a neurosteroid, such as Compound 1 or a pharmaceutically acceptable salt thereof. In some embodiments, the patient in need of a treatment of depression is a patient that is partially responsive to other antidepressant therapies. In certain embodiments, the patient in need of a treatment of depression is a patient that is partially responsive to treatment with selective serotonin reuptake inhibitors (SSRIs). In some embodiments, the patient in need of a treatment of depression is a patient with depression that is refractory to other therapies (i.e., treatment-resistant depression).

In some embodiments, the present disclosure provides methods of treating depression by administering a neurosteroid, such as Compound 1 or a pharmaceutically acceptable salt thereof, in combination with at least one additional antidepressant to a patient in need thereof. In certain embodiments, the additional antidepressant is selected from SSRIs, serotonin norepinephrine reuptake inhibitors, tricyclic antidepressants, monoamine oxidase inhibitors, mirtazapine, bupropion, lamotrigine, and atypical antipsychotics.

In some embodiments, the present disclosure provides methods of treating movement disorders, such as tremors (including essential tremor and any of the tremor conditions disclosed herein), by administering a neurosteroid, such as Compound 1 or a pharmaceutically acceptable salt thereof, in combination with a T-type calcium channel blocker to a patient in need thereof. In certain embodiments, the additional T-type calcium channel blocker is Compound 2:

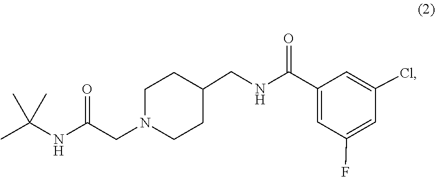 [SMILES: CC(C)(C)NC(=O)CN1CCC(CNC(=O)c2cc(F)cc(Cl)c2)CC1]

having the chemical name N-((1-(2-(tert-butylamino)-2-oxoethyl)piperidine-4-yl)methyl)-3chloro-5-fluorobenzamide, including pharmaceutically acceptable salts thereof (such as the HCl salt).

The present disclosure, among other things, also provides methods of treating not only movement disorders such as tremor, including essential tremor, but also comorbidities of tremor, such as depression, anxiety, and sleep disturbances and abnormalities (such as those discussed in “The Essential Tremors: Evolving Concepts of a Family of Diseases”, Elan D. Louis; “Sleep Disturbances in Essential Tremor and Parkinson Disease: A Polysomnographic Study”, Banu Ozen Barut, MD; Nida Tascilar, MD; Armagan Varo, MD) by administering a therapeutically effective amount of a neurosteroid, such as Compound 1 or a pharmaceutically acceptable salt thereof, to a patient in need thereof. In some embodiments, Compound 1 or a pharmaceutically acceptable salt thereof, is administered in combination with a therapeutically effective amount of Compound 2 or pharmaceutically acceptable salt thereof. In all instances of the present disclosure, “Compound 2” refers to both a non-deuterated form of Compound 2 and a deuterated form of Compound 2 as described herein. In some embodiments, disclosed herein is a composition comprising a therapeutically effective amount of Compound 1 or a pharmaceutically acceptable salt thereof and a therapeutically effective amount of Compound 2 or a pharmaceutically acceptable salt thereof.

The method comprises orally administering a daily dose of about 5 mg to about 120 mg of Compound 1 or a pharmaceutically acceptable salt thereof, optionally in combination with about 10 to about 150 mg of Compound 2 or a pharmaceutically acceptable salt thereof to a patient in need thereof.

In certain embodiments of the methods disclosed herein, including treating major depressive disorder (MDD), motor disorders, and/or musculoskeletal conditions in a patient in need thereof, the method comprises administering a therapeutically effective amount of Compound 1:

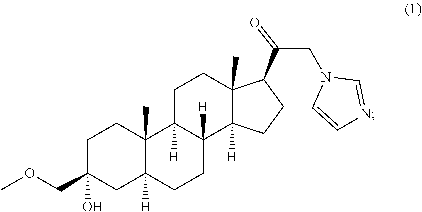

or a pharmaceutically acceptable salt thereof to a patient in need thereof.

Also disclosed herein are methods of reducing at least one side effect associated with administering a therapeutically effective amount of Compound 1 or a pharmaceutically acceptable salt thereof to a patient in need thereof, comprising administering Compound 1 in the evening, wherein the at least one side effect reduced is chosen from somnolence, sedation, dizziness, lightheadedness, fatigue, euphoria, feeling drunk, feeling abnormal, headache, cognitive disorder, disturbance of attention, memory impairment, slowed cognition, decreased alertness, constipation, or diarrhea. In certain embodiments, the at least one side effect associated with the administering of Compound 1 or a pharmaceutically acceptable salt are determined by the guidelines set forth in Guidance for Industry and Investigators: Safety Reporting Requirements for INDs and BA/BE Studies (2012), described in the ‘NIH-FDA Phase 2 and 3 IND/IDE Clinical Trial Protocol Template’, and reported with standardized terms according to the Medical Dictionary for Regulatory Activities.

Also disclosed herein are methods of treating a motor disorder in a patient in need thereof comprising orally administering a therapeutically effective amount of Compound 1 or a pharmaceutically acceptable salt thereof to a patient in need thereof. In certain embodiments, the motor disorder is chosen from cerebellar tremor, intention tremor, dystonic tremor, essential tremor, orthostatic tremor, Parkinsonian tremor, physiological tremor, psychogenic tremor, or rubral tremor, and in certain embodiments, the motor disorder is essential tremor.

Also disclosed herein are methods of treating a musculoskeletal condition in a patient in need thereof comprising orally administering a therapeutically effective amount of Compound 1 or a pharmaceutically acceptable salt thereof to a patient in need thereof. In certain embodiments, the musculoskeletal condition is chosen from fibromyalgia, polymyalgia, chronic fatigue syndrome, or systemic exertion intolerance disease, and in certain embodiments, the musculoskeletal condition is fibromyalgia.

In certain embodiments of all aspects, the methods disclosed herein further comprise co-administering Compound 2 or a pharmaceutically acceptable salt thereof.

In certain embodiments of all aspects, the administration is during the day, and in certain embodiments, the administration is in the evening. In certain embodiments wherein administration is in the evening, the evening is about two hours after the evening meal or about four hours after the evening meal. In certain embodiments, the evening is between about 5 p.m. and about 12 a.m., and in certain embodiments, the evening is about two hours prior to bedtime.

### BRIEF DESCRIPTION OF THE FIGURES

FIG. 1 is a graphical representation of the mean Compound 1 plasma concentration versus time for 15.0 mg daily administration of Compound 1 (Cohort 1), 30.0 mg daily administration of Compound 1 (Cohort 2), and a 60.0 mg daily administration of Compound 1 (Cohort 3), as described in Example 1.

FIG. 2 is a graphical representation of the mean Compound 1 steady state plasma concentration versus time for 15.0 mg daily administration of Compound 1 (Cohort 1), 30.0 mg daily administration of Compound 1 (Cohort 2), and a 60.0 mg daily administration of Compound 1 (Cohort 3), as described in Example 1.

FIG. 3 is a graphical representation of the Phase 2 clinical study protocol described in Example 2.

FIG. 4 is a graphical representation of the Compound 1 concentration over time following administration of an oral suspension of 45 mg or 60 mg of Compound 1 at 4 p.m. (four hours post-lunch) and at 8 p.m. (four hours post-dinner), as described in Example 3.

FIG. 5 is a graphical representation of the Compound 1 concentration over time following administration of 40 mg, 60 mg, and 80 mg of Compound 1 in the morning.

FIG. 6 is a graphical representation of the effect of administration time of 60 mg of Compound 1 on the mean (±standard deviation (SD)) Compound 1 concentration over time.

FIG. 7 shows a comparison of the GABA_{A} PAM quantitative EEG signals in the alpha-band and beta-band frequencies for Compound 1, allopregnanolone, and lorazepam.

FIG. 8 is a graphical representation of the effect of administering 30 mg or 60 mg of Compound 1 on normalized qEEG alpha and beta power over time.

FIG. 9 shows that Compound 1 dose-dependently inhibits harmaline-induced tremor in rats.

FIG. 10 shows the effects of administering Compound 1 and Compound 2 in combination on inhibiting tremor in the rat harmaline-induced tremor (rHIT) model.

FIG. 11 compares the effects of treatment with Compound 1 alone and the combination of Compound 1 and Compound 2 on the rHIT model.

FIG. 12A is a graphical representation of the Phase 2 clinical study protocol for Part A, described in Example 7.

FIG. 12B is a graphical representation of the Phase 2 clinical study protocol for Part B, described in Example 7.

### DETAILED DESCRIPTION OF THE DISCLOSURE

### Definitions

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The terms “administer,” “administering” or “administration” as used herein refer to either directly administering a compound or pharmaceutically acceptable salt or ester of the compound or a composition comprising the compound or pharmaceutically acceptable salt or ester of the compound to a patient.

The term “anxious distress” is used in this disclosure to mean the presence of at least two of the following symptoms during the majority of days of a major depressive episode or persistent depressive disorder (dysthymia): (1) Feeling keyed up or tense, (2) Feeling unusually restless, (3) Difficulty concentrating because of worry, (4) Fear that something awful may happen, and (5) Feeling that the individual might lose control of himself or herself. There are four severity categories of anxious distress: mild, moderate, moderate-severe and severe. “Mild anxious distress” is characterized by the presence of two of the five anxious distress symptoms. “Moderate anxious distress” is characterized by the presence of three of the five anxious distress symptoms. “Moderate-severe anxious distress” is characterized by the presence of four or five of the five anxious distress symptoms. “Severe anxious distress” is characterized by the presence of four or five of the five anxious distress symptoms and with motor agitation.

The term “child and adolescent depression” is used in this disclosure to mean child and adolescent depression as defined in the Diagnostic and Statistical Manual of Mental Disorders (DSM-5).

The term “child and adolescent suicidal ideation and behavior” is used in this disclosure to mean a child and adolescent with suicidal ideation and behavior as assessed by the Columbia-suicide severity rating scale (C-SSRS) and defined in the DSM-5.

The phrase “puberty” as used herein refers to puberty as defined by a validated staging system. In some embodiments, “puberty” refers to puberty as defined by the Stages of Reproductive Aging Workshop 10 Staging System (for female patients). In some embodiments, “puberty” refers to puberty as defined by the Tanner Stages Staging System.

The phrase “spermarche” and “spermarche transition” as used herein refers to spermarche and spermarche transition, respectively, as defined by a validated staging system. In some embodiments, “spermarche” and “spermarche transition” as used herein refers to spermarche and spermarche transition, respectively, as defined by the Tanner Stages Staging System.

The phrase “menarche” and “menarche transition” as used herein refers to menarche and menarche transition, respectively, as defined by a validated staging system. In some embodiments, “menarche” and “menarche transition” as used herein refers to menarche and menarche transition, respectively, as defined by the Stages of Reproductive Aging Workshop 10 Staging System. In some embodiments, “menarche” and “menarche transition” as used herein refers to menarche and menarche transition, respectively, as defined by the Tanner Stages Staging System.

The term “anxious major depressive disorder” (or anxious MDD) is used in this disclosure to mean a baseline 17-item Hamilton Depression Rating Scale (HAM-D) score ≥14 (excluding insomnia items) and a HAM-D anxiety/somatization score of ≥7.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical agent from one organ, or portion of the body, to another organ or portion of the body.

The term “insomnia” is used in this disclosure to mean insomnia as defined in the DSM-5.

The term “major depressive disorder” is used in this disclosure to mean major depressive disorder as defined in DSM-5. The term “moderate major depressive disorder” is used in this disclosure to mean major depressive disorder where the number of symptoms, intensity of symptoms, and/or functional impairment are between those specified in the DSM-5 for “mild” and “severe.” The term “severe major depressive disorder” is used in this disclosure to mean major depressive disorder where the number of symptoms is substantially in excess of that required to make the diagnosis, the intensity of the symptoms is seriously distressing and unmanageable, and the symptoms markedly interfere with social and occupational functioning.

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound, or a salt, solvate, or ester thereof, that, when administered to a patient, is capable of performing the intended result. For example, an effective amount of a salt of Compound 1 is that amount that is required to reduce at least one symptom of depression in a patient. The actual amount that comprises the “effective amount” or “therapeutically effective amount” will vary depending on several conditions including, but not limited to, the severity of the disorder, the size and health of the patient, and the route of administration.

The phrase “perimenopause” as used herein refers to early and late menopause transition stages as well as the early postmenopause.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salts” as used herein embraces pharmaceutically acceptable salts commonly used to form addition salts of free bases that are pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid.

The term “treating” as used herein regarding a patient, refers to improving at least one symptom of the patient's disorder. Treating can be improving, or at least partially ameliorating, a disorder. The term “therapeutic effect” as used herein refers to a desired or beneficial effect provided by the method and/or the composition. For example, the method for treating depression provides a therapeutic effect when the method reduces at least one symptom of depression in a patient.

The term “adjustment disorder” is used herein to mean adjustment disorder as defined by a validated system for diagnosing mental disorders. In some embodiments, the term “adjustment disorder” is used to mean adjustment disorder as defined by the DSM-5. In some embodiments, the term “adjustment disorder” is used to mean adjustment disorder as defined by code F43.2 of the 10th revision of the International Statistical Classification of Diseases and Related Health Problems (ICD-10). Similarly, the terms “adjustment disorder with depressed mood”, “adjustment disorder with anxiety”, and “adjustment disorder with mixed anxiety and depressed mood” are used in this disclosure to mean adjustment disorder with depressed mood, adjustment disorder with anxiety, and adjustment disorder with mixed anxiety and depressed mood, respectively, as defined in the DSM-5 or in the ICD-10.

Unless explicitly stated otherwise, a reference to Compound 1 means Compound 1, pharmaceutically acceptable salts of Compound 1, deuterated forms of Compound 1 (as described herein) and pharmaceutically acceptable salts of deuterated forms of Compound 1. A recitation of Compound 1, or pharmaceutically acceptable salts or deuterated forms thereof, or the like, similarly means Compound 1, pharmaceutically acceptable salts of Compound 1, deuterated forms of Compound 1 (as described herein) and pharmaceutically acceptable salts of deuterated forms of Compound 1.

Unless explicitly stated otherwise, a reference to Compound 2 means Compound 2, pharmaceutically acceptable salts of Compound 2, deuterated forms of Compound 2 (as described herein) and pharmaceutically acceptable salts of deuterated forms of Compound 2. A recitation of Compound 2, or pharmaceutically acceptable salts or deuterated forms thereof, or the like, similarly means Compound 2, pharmaceutically acceptable salts of Compound 2, deuterated forms of Compound 2 (as described herein) and pharmaceutically acceptable salts of deuterated forms of Compound 2.

Major Depressive Disorder (MDD) is a common psychiatric illness that causes disability and diminishes quality of life, depletes limited health care resources, increases morbidity and mortality, and increases the rates of substance abuse and suicide. The incidence of MDD in the United States and Australia is about 7% (American Psychiatric Association. (2013). Diagnostic and statistical manual of mental disorders (5th Ed.). Arlington, VA: American Psychiatric Publishing) and 5% (Australian Bureau of Statistics. (2008). National Survey of Mental Health and Wellbeing: Summary of results, 2007, cat no. 4326.0. Canberra: ABS), respectively. Current treatment options for MDD patients are limited and include SSRIs and serotonin norepinephrine uptake inhibitors (SNRIs). However, SSRIs and SNRIs may have liabilities in the context of treating MDD, such as a substantial delay in the onset of efficacy (several weeks) and high rates of treatment failure, e.g., about 33% of MDD patients fail to achieve full symptomatic remission despite multiple treatment regimens (Rush A J, et al. Acute and longer-term outcomes in depressed outpatients requiring one or several treatment steps: a STAR*D report. Am J Psychiatry. 2006; 163:1905-1917.).

Tremor is a term used to describe involuntary, at times rhythmic, muscle contractions and relaxation that can involve oscillations or twitching of one or more body parts (e.g., hands, arms, eyes, face, head, vocal folds, trunk, legs). Tremor can be divided into different classes, including cerebellar or intentional tremor, dystonic tremor, essential tremor, orthostatic tremor, Parkinsonian tremor, physiological tremor, psychogenic tremor, and rubral tremor. Neurophysiological rhythm generation may, in certain embodiments, be common in both seizure disorders, such as epilepsy, and movement disorders.

Cerebellar tremor or intentional tremor is characterized by a slow, broad tremor of the extremities that occurs after purposeful movement and can be caused by lesions in or damage to the cerebellum resulting from, for example, tumor, stroke, or a disease such as multiple sclerosis or an inherited degenerative disorder.

Dystonic tremors can affect any muscle in the body and occur in individuals affected by dystonia, which is a movement disorder characterized by irregular but sustained involuntary muscle contractions causing twisting and repetitive motions and/or painful and abnormal postures or positions.

Essential tremor or benign essential tremor is the most common type of tremor. Essential tremor can vary in severity (e.g., mild and nonprogressive in some patients and slowly progressive in others, starting on one side of the body but affecting both sides within a few years). Typically, the hands are most often affected, but the head, voice, tongue, legs, and trunk may also be involved. Tremor frequency may decrease as the person ages, but severity may increase. Tremors may be triggered and/or increased in severity by elevated emotional states, stress, fever, physical exhaustion, or low blood sugar. Tremor symptoms often evolve over time and can be both visible and persistent following onset.

Orthostatic tremor is characterized by fast (e.g., greater than 12 Hz) rhythmic muscle contractions in the legs and trunk immediately after standing. Cramps in the thighs and legs may make the patient shake uncontrollably if required to stand in one spot. Orthostatic tremor may also occur in patients with essential tremor.

Parkinsonian tremor is caused by damage to parts of the brain that control movement. Parkinsonian tremor is often a precursor to Parkinson's disease and typically manifests as a “pill-rolling” motion of the hands, but can also affect other parts of the body, such as the chin, lips, legs, and trunk. Onset of Parkinsonian tremors typically begins after age 60, and manifests as movements starting in one limb or on one side of the body and can progress to the other side of the body as well.

Physiological tremor can occur in “normal” individuals and can manifest in any voluntary muscle group. Physiological tremor can be caused by the use of certain drugs, alcohol withdrawal, or medical conditions, such as overactive thyroid or hypoglycemia. Physiological tremor typically has a frequency of about 10 Hz.

Psychogenic tremor or hysterical tremor can occur at rest or during postural or kinetic movement. Patients with psychogenic tremor may have a conversion disorder or another psychiatric disease.

Rubral tremor is characterized by coarse slow tremor which can be present at rest, at posture, and with intention, and is associated with conditions that affect the red nucleus in the midbrain, as well as classical unusual strokes.

Disturbances of the GABAergic system have been implicated in the development of depression and anxiety. A growing body of preclinical and clinical evidence supports the hypothesis that GABA_{A} hypofunction plays a role in the pathophysiology of depression and anxiety (Luscher B, Shen Q, Sahir N. The GABAergic deficit hypothesis of major depressive disorder. Mol Psychiatry. 2011; 16 (4):383-406). Supporting this hypothesis, drugs that enhance GABAergic function have shown some clinical benefit in the treatment of mood disorders. For example, benzodiazepines, which are positive allosteric modulators (PAMs) of the GABA_{A} receptor, are highly efficacious in the treatment of anxiety disorders. However, due to lack of efficacy, significant side effects (e.g., sedation), tolerance development, withdrawal symptoms upon cessation and a significant abuse potential, benzodiazepines are not recommended for the treatment of MDD. Therefore, there is a need for new treatment options for patients suffering from depression, such as MDD.

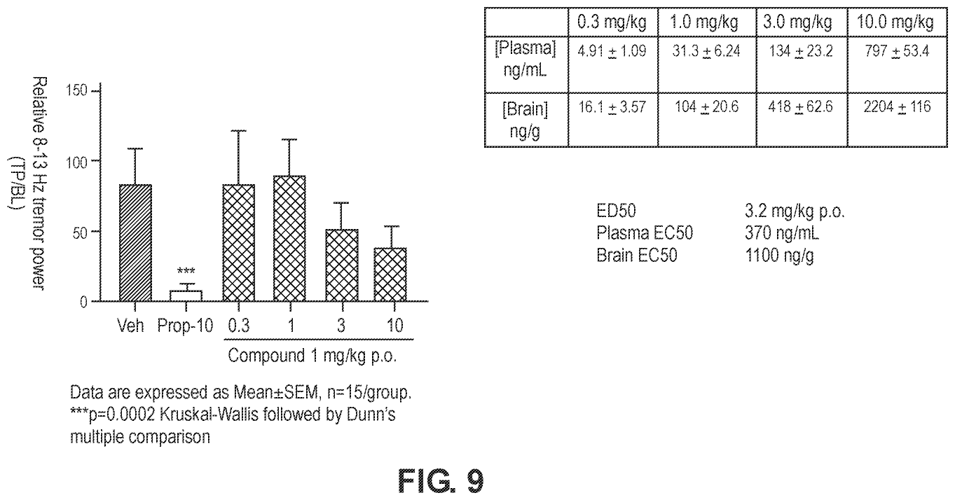

Neuroactive steroids (NASs) are a family of compounds (synthetic and naturally occurring) that affect neurophysiological functions through allosteric modulation of GABA_{A} receptors. The endogenous NASs allopregnanolone and pregnanolone are GABA_{A} PAMs that are dysregulated in mood disorders and show preclinical efficacy in animal models of anxiety and depression. NASs bind to a different binding site on the GABA_{A} receptor than benzodiazepines or the endogenous agonist GABA (Hosie A M, Wilkins M E, Da Silva H M A, Smart T G. Endogenous neurosteroids regulate GABA_{A} receptors through two discrete transmembrane sites. Nature. 2006; 444(7118):486-489.). Benzodiazepines exclusively potentiate GABA_{A} receptors that contain a gamma subunit, which are primarily localized at synapses. In contrast, NASs bind to alpha and beta subunits, which are present in a larger proportion of GABA_{A} receptors, resulting in broad activity at both synaptic and extrasynaptic sites. This differentiating pharmacology supports the utility of NAS for indications where benzodiazepines have not exhibited significant utility, such as MDD. NASs, such as Compound 1 (and physiologically acceptable salts or deuterated forms thereof), can also improve the symptoms of tremor, as shown in FIGS. 9-11.

In one aspect, the present disclosure provides a method of treating depression comprising administering an effective amount of a neuroactive steroid (or “neurosteroids”), for example Compound 1 or a pharmaceutically acceptable salt thereof, to a patient in need of such treatment. In another aspect, the present disclosure provides a method of treating a mood or affective disorder selected from perimenopause, generalized anxiety disorder, panic disorder, social anxiety disorder, acute stress disorder, post-traumatic stress disorder, specific phobia, and selective mutism comprising administering an effective amount of a neurosteroid, for example Compound 1 or a pharmaceutically acceptable salt thereof, to a patient in need of such treatment. In accordance with some embodiments of the present disclosure, at least about 15 mg, about 30 mg, about 45 mg, about 60 mg, about 75 mg, about 80 mg, about 85 mg, about 90 mg, about 95 mg, about 100 mg, about 105 mg, about 110 mg, about 115 mg or about 120 mg of a neurosteroid, for example Compound 1 or a pharmaceutically acceptable salt thereof, is administered.

In one aspect, the present disclosure provides a method of treating tremor comprising administering an effective amount of a neuroactive steroid (or “neurosteroids”), for example Compound 1 or a pharmaceutically acceptable salt thereof, to a patient in need of such treatment. In another aspect, the present disclosure provides a method of treating a mood or affective disorder, such as depression or anxiety associated with or comorbid with motor disorders, such as tremor, comprising administering an effective amount of a neurosteroid, for example Compound 1, a pharmaceutically acceptable salt thereof, optionally in combination with a T-type calcium channel blocker such as Compound 2 (or pharmaceutically acceptable salts thereof) to a patient in need of such treatment. In accordance with some embodiments of the present disclosure, at least about 5 mg, about 10 mg, about 15 mg, about 30 mg, about 45 mg, about 60 mg, about 75 mg, about 80 mg, about 85 mg, about 90 mg, about 95 mg, about 100 mg, about 105 mg, about 110 mg, about 115 mg or about 120 mg of a neurosteroid, for example Compound 1 or a pharmaceutically acceptable salt thereof. In other embodiments of the present disclosure, at least about 5 mg, about 10 mg, about 15 mg, about 30 mg, about 45 mg, about 60 mg, about 75 mg, about 80 mg, about 85 mg, about 90 mg, about 95 mg, about 100 mg, about 105 mg, about 110 mg, about 115 mg or about 120 mg of a neurosteroid, for example Compound 1 or a pharmaceutically acceptable salt thereof, is administered in combination with at least about 10 mg to about 200 mg of Compound 2 or a pharmaceutically acceptable salt thereof, including about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg of Compound 2 or a pharmaceutically acceptable salt thereof.

In another aspect, the present disclosure provides methods of administering a neurosteroid, for example Compound 1 or a pharmaceutically acceptable salt thereof, that reduces the adverse events (AEs) associated with administration of Compound 1 (e.g., somnolence, sedation, dizziness, lightheadedness, fatigue, euphoria, feeling drunk, feeling abnormal, headache, cognitive disorder, disturbance of attention, memory impairment, slowed cognition, decreased alertness, constipation, and diarrhea) compared to existing treatment methods. In some embodiments, the methods of the present disclosure reduce the incidence of AEs associated with administration of Compound 1. In some embodiments, the methods of the present disclosure reduce the severity of AEs associated with administration of Compound 1. As used herein, the incidence and severity of an AE may be determined according to guidelines set forth in Guidance for Industry and Investigators: Safety Reporting Requirements for INDs and BA/BE Studies (2012), described in the ‘NIH-FDA Phase 2 and 3 IND/IDE Clinical Trial Protocol Template’, and reported with standardized terms according to the Medical Dictionary for Regulatory Activities (MedDRA). Unexpectedly, it is disclosed herein that the administration of Compound 1 in the evening provides pharmacokinetic parameters that are associated with reduced AEs without decreasing the therapeutic effectiveness of Compound 1. Specifically, evening administration of Compound 1 reduces the maximum observed plasma concentration (Cₘₐₓ) and increases the time to maximum plasma concentration (tₘₐₓ) compared to morning administration of Compound 1 while providing a similar overall drug exposure (as determined by AUC_{inf}). The fact that evening administration of Compound 1 does not change the AUC_{inf} enables a patient to receive a similar total drug exposure over time while maintaining a lower Cₘₐₓ, which reduces AEs.

It is difficult to predict how the time of dosing impacts a particular drug's pharmacokinetics because multiple factors that impact drug pharmacokinetics are related to circadian variations, including protein binding, absorption from the gastrointestinal tract, the blood flow fraction an organ receives, and liver enzyme activity, as well as the physicochemical properties of the particular drug.

The difficulty in predicting the effect of dosing time on the pharmacokinetics and therapeutic effect of a therapeutic is exemplified by amlodipine besylate. Like Compound 1, amlodipine is lipophilic. Unlike Compound 1, evening administration of amlodipine besylate is associated with a reduced Tₘₐₓ and increased Cₘₐₓ compared to morning dosing. Khodadoustan et al. (2017). Clinical and Experimental Hypertension, 39:6, 520-526. In contrast, and unexpectedly, evening dosing of Compound 1 provides an increase in Tₘₐₓ and decrease in Cₘₐₓ, which is associated with reduced side effects. Thus, patients treated with Compound 1 according to the methods described herein experience a therapeutic benefit with reduced AEs.

In some embodiments, the present methods employ a neuroactive steroid. The neuroactive steroid as employed in the present methods can form a part of a pharmaceutical composition by combining a neuroactive steroid, or a pharmaceutically acceptable salt thereof, with a pharmaceutically acceptable carrier. In certain embodiments, the neuroactive steroid is selected from the group consisting of pregnanolone, allopregnanolone, allotetrahydrodeoxycorticosterone, ganaxolone, alphaxolone, alphadolone, hydroxydione, minaxolone, Althesin, Renanolone, SAGE-324, SAGE-217 (3α-hydroxy-30-methyl-21-(4-cyano-1H-pyrazol-1′-yl)-19-nor-50-pregnan-20-one), and any neuroactive steroid as described in U.S. Publication No. 2017/0240589, U.S. Pat. No. 10,857,163, U.S. Publication No. 2021/0093648, WO 2019/154247, WO 2019/154257, and WO 2021/023213, which are hereby incorporated by reference in their entirety for all purposes. In other embodiments, the preceding neuroactive steroids are administered in combination with a therapeutically effective amount of Compound 2.

### Compound 1

In some embodiments, the neuroactive steroid is Compound 1. Compound 1 as employed in the present methods can form a part of a pharmaceutical composition by combining Compound 1, or a pharmaceutically acceptable salt thereof, with a pharmaceutically acceptable carrier. Additionally, the compositions can include an additive selected from the group consisting of adjuvants, excipients, diluents, release-modifying agents, and stabilizers. The composition can be an immediate release formulation, a delayed release formulation, a sustained release formulation, or an extended-release formulation.

3α-Hydroxy-3β-methoxymethyl-21-(1′-imidazolyl)-5α-pregnan-20-one (Compound 1) is a synthetic neuroactive steroid. The structural formula of Compound 1 appears below.

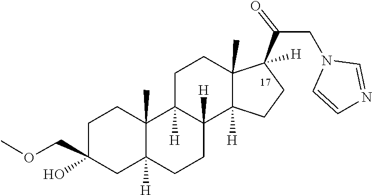

Compound 1 is a neuroactive steroid GABA-A positive allosteric modulator (PAM) with high potency similar to clinical stage neuroactive steroids (allopregnanolone, ganaxolone, SAGE-217, alphaxolone).

The synthesis of Compound 1 is described in U.S. Publication Nos. 2004/034002 and 2009/0118248, which are hereby incorporated by reference in their entirety for all purposes. A crystalline polymorph of Compound 1 free base is described in U.S. Publication No. 2006/0074059, and pharmaceutical compositions containing Compound 1 are described in U.S. Publication No. 2009/0131383 and PCT Application No. PCT/US2022/016612, which are hereby incorporated by reference in their entirety for all purposes.

In some embodiments, the Compound 1 used in the formulations and methods of the present disclosure is a pharmaceutically acceptable salt of Compound 1. Salts of Compound 1 and polymorphs thereof are described in U.S. Pat. No. 10,562,930, which is hereby incorporated by reference in its entirety. In some embodiments, the pharmaceutically acceptable salt of Compound 1 used in the formulations and methods of the present disclosure is selected from the group consisting of hydrobromide, citrate, hemicitrate, malate, mesylate, phosphate, tartrate, hydrochloride, tosylate, glucuronate, ethanesulfonate, fumarate, sulfate, napthalene-2-sulfonate, ascorbate, oxalate, napthalene-1,5-disulfonate, malonate, aminosalicylate, benzenesulfonate, isethionate, gentistate, 1-hydroxy-2-napthoate, dichloroacetate, cyclamate, and ethane-1,2-disulfonate salts. In certain embodiments, the salt of Compound 1 is Compound 1 Hydrobromide. In certain embodiments, the salt of Compound 1 is Compound 1 Citrate. In certain embodiments, the salt of Compound 1 is Compound 1 L-Malate. In certain embodiments, the salt of Compound 1 is Compound 1 Mesylate. In certain embodiments, the salt of Compound 1 is Compound 1 Phosphate. In certain embodiments, the salt of Compound 1 is Compound 1 L(+)-Tartrate. In certain embodiments, the salt of Compound 1 is Compound 1 Hydrochloride. In certain embodiments, the salt of Compound 1 is Compound 1 Tosylate. In certain embodiments, the salt of Compound 1 is Compound 1 Glucuronate. In certain embodiments, the salt of Compound 1 is Compound 1 Ethanesulfonate. In certain embodiments, the salt of Compound 1 is Compound 1 hemicitrate.

The structural formula of Compound 2 appears below.

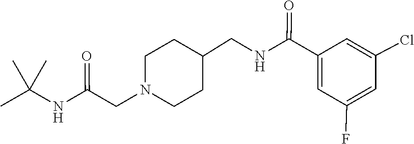 [SMILES: CC(C)(C)NC(=O)CN1CCC(CNC(=O)c2cc(F)cc(Cl)c2)CC1]

The synthesis of Compound 2, salts thereof, and pharmaceutical compositions thereof are described in U.S. Pat. No. 8,377,968, which is incorporated herein by reference in its entirety for all purposes. The safety profile, efficacy, tolerability, and pharmacokinetics of Compound 2 in modified release formulations with and without titration has been assessed, as described in PCT Publication WO 2021/222342, which is incorporated by reference herein its entirety.

### Formulations

The methods of the present disclosure can employ various formulations for administration to patients, e.g., humans in unit dosage forms, such as tablets, capsules, pills, powders, granules, sterile parenteral solutions or suspensions (e.g., intramuscular (IM), subcutaneous (SC) and intravenous (IV)), transdermal patches, and oral solutions or suspensions, and oil-water emulsions containing suitable quantities of Compound 1 or a pharmaceutically acceptable salt thereof.

Oral pharmaceutical dosage forms can be either solid or liquid. The solid dosage forms can be tablets, capsules, granules, films (e.g., buccal films) and bulk powders. Types of oral tablets include compressed, chewable lozenges and tablets, which can be enteric-coated, sugar-coated or film-coated. Capsules can be hard or soft gelatin capsules, while granules and powders can be provided in non-effervescent or effervescent form with the combination of other ingredients known to those skilled in the art. In some embodiments, the present oral dosage forms may include orally disintegrating tablets.

Pharmaceutically acceptable carriers utilized in tablets include binders, lubricants, diluents, disintegrating agents, coloring agents, flavoring agents, and wetting agents.

Liquid oral dosage forms include aqueous solutions, emulsions, suspensions, solutions and/or suspensions reconstituted from non-effervescent granules and effervescent preparations reconstituted from effervescent granules.

Aqueous solutions include, for example, elixirs and syrups. Emulsions can be either oil-in-water or water-in-oil. Elixirs are clear, sweetened, hydroalcoholic preparations. Pharmaceutically acceptable carriers used in elixirs include solvents. Syrups can be concentrated aqueous solutions of a sugar, for example, sucrose, and can contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions can use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substance used in effervescent granules, to be reconstituted into a liquid oral dosage form, can include organic acids and a source of carbon dioxide. Coloring and flavoring agents can be used in all of the above dosage forms.

In some embodiments, the present disclosure provides a pharmaceutical composition comprising a salt of Compound 1. In some embodiments, the salt of Compound 1 is Compound 1 Hydrobromide, Compound 1 Citrate, Compound 1 L-Malate, Compound 1 Mesylate, Compound 1 Phosphate, Compound 1 L(+)-Tartrate, Compound 1 Hydrochloride, Compound 1 Tosylate, Compound 1 Glucuronate, Compound 1 Ethanesulfonate, or Compound 1 hemicitrate.

In some embodiments, the present disclosure provides a pharmaceutical composition comprising Compound 1 or a pharmaceutically acceptable salt thereof and Compound 2 or a pharmaceutically acceptable salt thereof.

### Co-Therapy

While the compositions can be administered as the sole active pharmaceutical ingredient (i.e., Compound 1 or a pharmaceutically acceptable salt thereof) or sole active anti-depressant ingredient in the methods described herein, in some embodiments they can also be used in combination with one or more ingredients which are known to be therapeutically effective against depression and/or compliment the antidepressant effect of the Compound 1 ingredient. In some embodiments Compound 1 or a pharmaceutically acceptable salt thereof can also be used in combination with one or more ingredients which are known to be therapeutically effective against tremor and/or compliment the anti-tremor effect of the Compound 1 ingredient.

For example, in some embodiments, the present methods can employ Compound 1 or a pharmaceutically acceptable salt thereof in conjunction with one or more additional active agents such as T-type calcium channel blockers. In a particular embodiment, the T-type calcium channel blocker is Compound 2 or a pharmaceutically acceptable salt thereof.

In some embodiments, Compound 1 or a pharmaceutically acceptable salt thereof is administered in combination with a T-type calcium channel blocker, e.g., co-formulated or administered separately. In some embodiments, Compound 1 or a pharmaceutically acceptable salt thereof is administered in conjunction with one or more T-type calcium channel blockers including Compound 2 or pharmaceutically acceptable salts thereof, or one or more of ethosuximide, trimethadione, zonisamide, amlodipine, aranidipine, azelnidipine, barnidipine, benidipine, efonidipine, mibefradil, nicardipine, nimodipine, lomerizine, and pimozide.

For example, in some embodiments, the present methods can employ Compound 1, or a pharmaceutically acceptable salt thereof, in conjunction with one or more additional anti-antidepressants agents.

In some embodiments, Compound 1 or a pharmaceutically acceptable salt thereof is administered in combination with an additional anti-depressant agent, e.g., co-formulated or administered separately. In some embodiments, Compound 1 or a pharmaceutically acceptable salt thereof is administered in conjunction with one or more SSRIs, SNRIs, tricyclic antidepressants, monoamine oxidase inhibitors, mirtazapine, bupropion, lamotrigine atypical antipsychotics, or combinations thereof. In some embodiments, Compound 1 or a pharmaceutically acceptable salt thereof is administered in combination with electroconvulsive therapy (ECT). In some embodiments, Compound 1 or a pharmaceutically acceptable salt thereof is administered in combination with transcranial magnetic stimulation (TMS).

In some embodiments, Compound 1 or a pharmaceutically acceptable salt thereof is administered in conjunction with one or more SSRIs. In certain embodiments, the one or more SSRIs is selected from the group consisting of fluoxetine, escitalopram, citalopram, sertraline, and paroxetine.

In some embodiments, Compound 1 or a pharmaceutically acceptable salt thereof is administered in conjunction with one or more SNRIs. In certain embodiments, the one or more serotonin norepinephrine reuptake inhibitors is selected from the group consisting of venlafaxine and duloxetine.

In some embodiments, Compound 1 or a pharmaceutically acceptable salt thereof is administered in conjunction with one or more tricyclic antidepressants. In certain embodiments, the one or more tricyclic antidepressants is selected from the group consisting of amitriptyline, imipramine, and nortriptyline.

In some embodiments, Compound 1 or a pharmaceutically acceptable salt thereof is administered in conjunction with one or more monoamine oxidase inhibitors. In certain embodiments, the one or more monoamine oxidase inhibitors is selected from the group consisting of phenelzine and tranylcypromine.

In some embodiments, Compound 1 or a pharmaceutically acceptable salt thereof is administered in conjunction with one or more atypical antipsychotics. In certain embodiments, the one or more atypical antipsychotics is selected from the group consisting of lurasidone, aripiprazole, risperidone, olanzapine, quetiapine, ziprasidone, clozapine, iloperidone, paliperidone, asenapine, and olanzapine/fluoxetine.

### Dosing

In the following embodiments, the dose, dosing frequency, and dosing schedule of a neuroactive steroid is expressed in terms of the dose (e.g., dose of Compound 1, or equivalent dose of a pharmaceutically acceptable salt thereof) administered. Also disclosed herein are embodiments where the neurosteroid is selected from the group consisting of pregnanolone, allopregnanolone, allotetrahydrodeoxycorticosterone, ganaxolone, alphaxolone, alphadolone, hydroxydione, minaxolone, Althesin, Renanolone, SAGE-324, SAGE-217 (3α-hydroxy-30-methyl-21-(4-cyano-1H-pyrazol-1′-yl)-19-nor-50-pregnan-20-one), and any neuroactive steroid as described in U.S. Publication No. 2017/0240589. In the embodiments disclosed herein, any neurosteroid disclosed herein can be substituted for Compound 1 or pharmaceutically acceptable salts thereof.

The present disclosure provides methods for treating depression by administering an effective amount of a neurosteroid, such as Compound 1 or a pharmaceutically acceptable salt thereof, to a patient in need thereof. An effective amount is an amount sufficient to eliminate or significantly reduce depression symptoms or to alleviate those symptoms (e.g., reduce the symptoms, such as depressed mood, compared to the symptoms present prior to treatment).

The present disclosure provides methods for treating tremor by administering an effective amount of a neurosteroid, such as Compound 1 or a pharmaceutically acceptable salt thereof, to a patient in need thereof. An effective amount is an amount sufficient to eliminate or significantly reduce tremor symptoms or to alleviate those symptoms (e.g., reduce the symptoms, such as frequency, severity, or magnitude of tremor, compared to the symptoms present prior to treatment).

According to some embodiments of the present disclosure, administering a neurosteroid such as Compound 1 or a pharmaceutically acceptable salt thereof provides a statistically significant therapeutic effect. In one embodiment, the statistically significant therapeutic effect is determined based on one or more standards or criteria provided by one or more regulatory agencies in the United States, e.g., FDA or other countries (such as Australia). In some embodiments, the statistically significant therapeutic effect is determined based on results obtained from regulatory agency approved clinical trial set up and/or procedure.

In some embodiments, the statistically significant therapeutic effect is determined based on a patient population of at least 20, 50, 60, 100, 200, 300, 400, 500, 600, 700, 800, 900, 1000, or 2000. In some embodiments, the statistically significant therapeutic effect is determined based on data obtained from randomized and double-blinded clinical trial set up. In some embodiments, the statistically significant therapeutic effect is determined based on data with a p-value of less than or equal to about 0.05, 0.04, 0.03, 0.02, or 0.01. In some embodiments, the statistically significant therapeutic effect is determined based on data with a confidence interval greater than or equal to 95%, 96%, 97%, 98%, or 99%.

In some embodiments, the statistically significant therapeutic effect is determined by a randomized double-blind clinical trial of patients treated with a neurosteroid, such as Compound 1 or a pharmaceutically acceptable salt thereof, and optionally in combination with standard care. In some embodiments, the statistically significant therapeutic effect is determined by a randomized clinical trial and using Hamilton Depression Rating Scale (HAM-D) as primary efficacy parameter and optionally in combination with any other commonly accepted criteria for depression assessment.

In some embodiments, the statistically significant therapeutic effect is determined by a randomized double-blind clinical trial of patients treated with a neurosteroid, such as Compound 1 or a pharmaceutically acceptable salt thereof and optionally in combination with standard care. In some embodiments, the statistically significant therapeutic effect is determined by a randomized clinical trial and using the Tremor Rating Assessment Scale (TETRAS) as primary efficacy parameter(s) and optionally in combination with any other commonly accepted criteria for tremor assessment.

In general, statistical analysis can include any suitable method permitted by a regulatory agency, e.g., FDA in the US or Europe or any other country. In some embodiments, statistical analysis includes non-stratified analysis, log-rank analysis, e.g., from Kaplan-Meier, Jacobson-Truax, Gulliken-Lord-Novick, Edwards-Nunnally, Hageman-Arrindel and Hierarchical Linear Modeling (HLM), and Cox regression analysis.

According to the present disclosure, a neurosteroid, such as Compound 1 or a pharmaceutically acceptable salt thereof, is administered on a once-a-day or twice-a-day basis to provide effective relief of the symptoms of depression (for example, major depressive disorder, major depressive disorder with suicidal risk, clinical depression, postnatal or postpartum depression, treatment resistant postpartum depression, perimenopausal depression, child and adolescent depression, premenstrual dysphoric disorder (PMDD), atypical depression, melancholic depression, Psychotic Major Depression (PMD), catatonic depression, Seasonal Affective Disorder (SAD), persistent depressive disorder (dysthymia), double depression, Depressive Personality Disorder (DPD), Recurrent Brief Depression (RBD), minor depressive disorder, bipolar disorder or manic depressive disorder, bipolar depression with suicidal risk, post-traumatic stress disorders, depression caused by chronic medical conditions, depressive disorder due to another medical condition, treatment-resistant depression, refractory depression, substance/medication induced depressive disorder, depression with anxiety, suicidality, suicidal ideation, or suicidal behavior) or a mood or affective disorder selected from perimenopause, generalized anxiety disorder, panic disorder, social anxiety disorder, acute stress disorder, post-traumatic stress disorder, specific phobia, and selective mutism. In some embodiments, the neurosteroid. such as Compound 1 or a pharmaceutically acceptable salt thereof, is administered on a once-a-day or twice-a-day basis to provide effective relief of the symptoms of acute stress disorder. In some embodiments, Compound 1 or a pharmaceutically acceptable salt thereof is administered on a once-a-day or twice-a-day basis to provide effective relief of the symptoms of post-traumatic stress disorder.

According to the present disclosure, a neurosteroid, such as Compound 1 or a pharmaceutically acceptable salt thereof, is administered on a once-a-day or twice-a-day basis to provide effective relief of the symptoms of tremor (for example, essential tremor, medication-induced postural tremor, postural tremor, rest tremor, intentional tremor, orthostatic tremor, cerebellar tremor, Parkinsonian tremor, physiological tremor, psychogenic tremor, rubral tremor, and other tremors described herein). In some embodiments, the neurosteroid, such as Compound 1 or a pharmaceutically acceptable salt thereof, is administered on a once-a-day or twice-a-day basis to provide effective relief of the symptoms of essential tremor.

In some embodiments, a total daily dose of any neurosteroid described herein, for example Compound 1 or an equivalent amount of a pharmaceutically acceptable salt thereof is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, and about 120 mg. In certain embodiments, the total daily dose of SAGE-217 is from about 10 mg to about 60 mg, including about 10 mg, about 15 mg, about 20 mg, about 25 mg, about 30 mg, about 35 mg, about 40 mg, about 45 mg, about 50 mg, about 55 mg, and about 60 mg, including all ranges there between. In some embodiments, the total daily dose of SAGE-217 is from about 20 mg to about 60 mg, including about 20 mg, about 25 mg, about 30 mg, about 35 mg, about 40 mg, about 45 mg, about 50 mg, about 55 mg, or about 60 mg, including all doses and ranges there between. In some embodiments, the total daily dose of SAGE-217 is about 20 mg. In some embodiments, the total daily dose of SAGE-217 is about 30 mg. In some embodiments, the total daily dose of SAGE-217 is about 50 mg.

In certain embodiments, the total daily dose of allopregnanolone is from about 25 mg to about 400 mg, including about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, and about 400 mg.

In certain embodiments, the total daily dose of Compound 1 or a pharmaceutically acceptable salt thereof is from about 5 mg to about 120 mg, including about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, and about 120 mg, including all ranges there between. In certain embodiments, the total daily dose of Compound 1 or a pharmaceutically acceptable salt thereof is from about 15 mg to about 60 mg. In certain embodiments, the total daily dose of Compound 1 or a pharmaceutically acceptable salt thereof is from about 15 mg to about 80 mg. In certain embodiments, the total daily dose of Compound 1 or a pharmaceutically acceptable salt thereof is from about 15 mg to about 100 mg. In certain embodiments, the total daily dose of Compound 1 or a pharmaceutically acceptable salt thereof is from about 45 mg to about 60 mg. In certain embodiments, the total daily dose of Compound 1 or a pharmaceutically acceptable salt thereof is from about 45 mg to about 80 mg.

In the embodiments described herein, reference is made to the dose of Compound 1 or a pharmaceutically acceptable salt thereof for the treatment of tremor (which includes essential tremor or comorbid depression or anxiety associated with tremor).

In the embodiments described herein, reference is made to the dose of Compound 1 or a pharmaceutically acceptable salt thereof for the treatment of depression (which includes acute treatment of depression and chronic treatment of depression). However, the present disclosure contemplates the disclosed doses of any of the neurosteroids disclosed herein for the treatment of a mood or affective disorder selected from perimenopause, generalized anxiety disorder, panic disorder, social anxiety disorder, acute stress disorder, post-traumatic stress disorder, specific phobia, and selective mutism.

In some embodiments, the total daily dose of Compound 1 or a pharmaceutically acceptable salt thereof is at least about 5 mg a day for the treatment of depression. In some embodiments, the total daily dose of Compound 1 or a pharmaceutically acceptable salt thereof is at least about 10 mg a day for the treatment of depression. In some embodiments, the total daily dose of Compound 1 or a pharmaceutically acceptable salt thereof is at least about 15 mg a day for the treatment of depression. In some embodiments, the total daily dose of Compound 1 or a pharmaceutically acceptable salt thereof is at least about 20 mg a day for the treatment of depression. In some embodiments, the total daily dose of Compound 1 or a pharmaceutically acceptable salt thereof is at least about 25 mg a day for the treatment of depression. In some embodiments, the total daily dose of Compound 1 or a pharmaceutically acceptable salt thereof is at least about 30 mg a day for the treatment of depression. In some embodiments, the total daily dose of Compound 1 or a pharmaceutically acceptable salt thereof is at least about 35 mg a day for the treatment of depression. In some embodiments, the total daily dose of Compound 1 or a pharmaceutically acceptable salt thereof is at least about 40 mg a day for the treatment of depression. In some embodiments, the total daily dose of Compound 1 or a pharmaceutically acceptable salt thereof is at least about 45 mg a day for the treatment of depression. In some embodiments, the total daily dose of Compound 1 or a pharmaceutically acceptable salt thereof is at least about 50 mg a day for the treatment of depression. In some embodiments, the total daily dose of Compound 1 or a pharmaceutically acceptable salt thereof is at least about 55 mg a day for the treatment of depression. In some embodiments, the total daily dose of Compound 1 or a pharmaceutically acceptable salt thereof is at least about 60 mg a day for the treatment of depression. In some embodiments, the total daily dose of Compound 1 or a pharmaceutically acceptable salt thereof is at least about 65 mg a day for the treatment of depression. In some embodiments, the total daily dose of Compound 1 or a pharmaceutically acceptable salt thereof is at least about 70 mg a day for the treatment of depression. In some embodiments, the total daily dose of Compound 1 or a pharmaceutically acceptable salt thereof is at least about 75 mg a day for the treatment of depression. In some embodiments, the total daily dose of Compound 1 or a pharmaceutically acceptable salt thereof is at least about 80 mg a day for the treatment of depression. In some embodiments, the total daily dose of Compound 1 or a pharmaceutically acceptable salt thereof is at least about 85 mg a day for the treatment of depression. In some embodiments, the total daily dose of Compound 1 or a pharmaceutically acceptable salt thereof is at least about 90 mg a day for the treatment of depression. In some embodiments, the total daily dose of Compound 1 or a pharmaceutically acceptable salt thereof is at least about 95 mg a day for the treatment of depression. In some embodiments, the total daily dose of Compound 1 or a pharmaceutically acceptable salt thereof is at least about 100 mg a day for the treatment of depression. In some embodiments, the total daily dose of Compound 1 or a pharmaceutically acceptable salt thereof is at least about 105 mg a day for the treatment of depression. In some embodiments, the total daily dose of Compound 1 or a pharmaceutically acceptable salt thereof is at least about 110 mg a day for the treatment of depression. In some embodiments, the total daily dose of Compound 1 or a pharmaceutically acceptable salt thereof is at least about 115 mg a day for the treatment of depression. In some embodiments, the total daily dose of Compound 1 or a pharmaceutically acceptable salt thereof is at least about 120 mg a day for the treatment of depression.

In some embodiments, the total daily dose of Compound 1 or a pharmaceutically acceptable salt thereof is about 5 mg a day for the treatment of depression. In some embodiments, the total daily dose of Compound 1 or a pharmaceutically acceptable salt thereof is about 10 mg a day for the treatment of depression. In some embodiments, the total daily dose of Compound 1 or a pharmaceutically acceptable salt thereof is about 15 mg a day for the treatment of depression. In some embodiments, the total daily dose of Compound 1 or a pharmaceutically acceptable salt thereof is about 20 mg a day for the treatment of depression. In some embodiments, the total daily dose of Compound 1 or a pharmaceutically acceptable salt thereof is about 25 mg a day for the treatment of depression. In some embodiments, the total daily dose of Compound 1 or a pharmaceutically acceptable salt thereof is about 30 mg a day for the treatment of depression. In some embodiments, the total daily dose of Compound 1 or a pharmaceutically acceptable salt thereof is about 35 mg a day for the treatment of depression. In some embodiments, the total daily dose of Compound 1 or a pharmaceutically acceptable salt thereof is about 40 mg a day for the treatment of depression. In some embodiments, the total daily dose of Compound 1 or a pharmaceutically acceptable salt thereof is about 45 mg a day for the treatment of depression. In some embodiments, the total daily dose of Compound 1 or a pharmaceutically acceptable salt thereof is about 50 mg a day for the treatment of depression. In some embodiments, the total daily dose of Compound 1 or a pharmaceutically acceptable salt thereof is about 55 mg a day for the treatment of depression. In some embodiments, the total daily dose of Compound 1 or a pharmaceutically acceptable salt thereof is about 60 mg a day for the treatment of depression. In some embodiments, the total daily dose of Compound 1 or a pharmaceutically acceptable salt thereof is about 65 mg a day for the treatment of depression. In some embodiments, the total daily dose of Compound 1 or a pharmaceutically acceptable salt thereof is about 70 mg a day for the treatment of depression. In some embodiments, the total daily dose of Compound 1 or a pharmaceutically acceptable salt thereof is about 75 mg a day for the treatment of depression. In some embodiments, the total daily dose of Compound 1 or a pharmaceutically acceptable salt thereof is about 80 mg a day for the treatment of depression. In some embodiments, the total daily dose of Compound 1 or a pharmaceutically acceptable salt thereof is about 85 mg a day for the treatment of depression. In some embodiments, the total daily dose of Compound 1 or a pharmaceutically acceptable salt thereof is about 90 mg a day for the treatment of depression. In some embodiments, the total daily dose of Compound 1 or a pharmaceutically acceptable salt thereof is about 95 mg a day for the treatment of depression. In some embodiments, the total daily dose of Compound 1 or a pharmaceutically acceptable salt thereof is about 100 mg a day for the treatment of depression. In some embodiments, the total daily dose of Compound 1 or a pharmaceutically acceptable salt thereof is about 105 mg a day for the treatment of depression. In some embodiments, the total daily dose of Compound 1 or a pharmaceutically acceptable salt thereof is about 110 mg a day for the treatment of depression. In some embodiments, the total daily dose of Compound 1 or a pharmaceutically acceptable salt thereof is about 115 mg a day for the treatment of depression. In some embodiments, the total daily dose of Compound 1 or a pharmaceutically acceptable salt thereof is about 120 mg a day for the treatment of depression.

In some embodiments, about 5 mg of Compound 1 or a pharmaceutically acceptable salt thereof once a day is selected to provide a substantial reduction in depression. In some embodiments, about 5 mg of Compound 1 or a pharmaceutically acceptable salt thereof twice a day is selected to provide a substantial reduction in depression. In some embodiments, about 10 mg of Compound 1 or a pharmaceutically acceptable salt thereof once a day is selected to provide a substantial reduction in depression. In some embodiments, about 10 mg of Compound 1 or a pharmaceutically acceptable salt thereof twice a day is selected to provide a substantial reduction in depression. In some embodiments, about 15 mg of Compound 1 or a pharmaceutically acceptable salt thereof once a day is selected to provide a substantial reduction in depression. In some embodiments, about 15 mg of Compound 1 or a pharmaceutically acceptable salt thereof twice a day is selected to provide a substantial reduction in depression. In some embodiments, about 20 mg of Compound 1 or a pharmaceutically acceptable salt thereof once a day is selected to provide a substantial reduction in depression. In some embodiments, about 20 mg of Compound 1 or a pharmaceutically acceptable salt thereof twice a day is selected to provide a substantial reduction in depression. In some embodiments, about 25 mg of Compound 1 or a pharmaceutically acceptable salt thereof once a day is selected to provide a substantial reduction in depression. In some embodiments, about 25 mg of Compound 1 or a pharmaceutically acceptable salt thereof twice a day is selected to provide a substantial reduction in depression. In some embodiments, about 30 mg of Compound 1 or a pharmaceutically acceptable salt thereof once a day is selected to provide a substantial reduction in depression. In some embodiments, about 30 mg of Compound 1 or a pharmaceutically acceptable salt thereof twice a day is selected to provide a substantial reduction in depression. In some embodiments, about 30 mg of Compound 1 or a pharmaceutically acceptable salt thereof once a day is selected to provide a substantial reduction in depression. In some embodiments, about 30 mg of Compound 1 or a pharmaceutically acceptable salt thereof twice a day is selected to provide a substantial reduction in depression. In some embodiments, about 35 mg of Compound 1 or a pharmaceutically acceptable salt thereof once a day is selected to provide a substantial reduction in depression. In some embodiments, about 35 mg of Compound 1 or a pharmaceutically acceptable salt thereof twice a day is selected to provide a substantial reduction in depression. In some embodiments, about 40 mg of Compound 1 or a pharmaceutically acceptable salt thereof once a day is selected to provide a substantial reduction in depression. In some embodiments, about 40 mg of Compound 1 or a pharmaceutically acceptable salt thereof twice a day is selected to provide a substantial reduction in depression. In some embodiments, about 45 mg of Compound 1 or a pharmaceutically acceptable salt thereof once a day is selected to provide a substantial reduction in depression. In some embodiments, about 45 mg of Compound 1 or a pharmaceutically acceptable salt thereof twice a day is selected to provide a substantial reduction in depression. In some embodiments, about 50 mg of Compound 1 or a pharmaceutically acceptable salt thereof once a day is selected to provide a substantial reduction in depression. In some embodiments, about 50 mg of Compound 1 or a pharmaceutically acceptable salt thereof twice a day is selected to provide a substantial reduction in depression. In some embodiments, about 55 mg of Compound 1 or a pharmaceutically acceptable salt thereof once a day is selected to provide a substantial reduction in depression. In some embodiments, about 55 mg of Compound 1 or a pharmaceutically acceptable salt thereof twice a day is selected to provide a substantial reduction in depression. In some embodiments, about 60 mg of Compound 1 or a pharmaceutically acceptable salt thereof once a day is selected to provide a substantial reduction in depression. In some embodiments, about 60 mg of Compound 1 or a pharmaceutically acceptable salt thereof twice a day is selected to provide a substantial reduction in depression. In some embodiments, about 65 mg of Compound 1 or a pharmaceutically acceptable salt thereof once a day is selected to provide a substantial reduction in depression. In some embodiments, about 70 mg of Compound 1 or a pharmaceutically acceptable salt thereof once a day is selected to provide a substantial reduction in depression. In some embodiments, about 75 mg of Compound 1 or a pharmaceutically acceptable salt thereof once a day is selected to provide a substantial reduction in depression. In some embodiments, about 80 mg of Compound 1 or a pharmaceutically acceptable salt thereof once a day is selected to provide a substantial reduction in depression. In some embodiments, about 85 mg of Compound 1 or a pharmaceutically acceptable salt thereof once a day is selected to provide a substantial reduction in depression. In some embodiments, about 90 mg of Compound 1 or a pharmaceutically acceptable salt thereof once a day is selected to provide a substantial reduction in depression. In some embodiments, about 95 mg of Compound 1 or a pharmaceutically acceptable salt thereof once a day is selected to provide a substantial reduction in depression. In some embodiments, about 100 mg of Compound 1 or a pharmaceutically acceptable salt thereof once a day is selected to provide a substantial reduction in depression. In some embodiments, about 105 mg of Compound 1 or a pharmaceutically acceptable salt thereof once a day is selected to provide a substantial reduction in depression. In some embodiments, about 110 mg of Compound 1 or a pharmaceutically acceptable salt thereof once a day is selected to provide a substantial reduction in depression. In some embodiments, about 115 mg of Compound 1 or a pharmaceutically acceptable salt thereof once a day is selected to provide a substantial reduction in depression. In some embodiments, about 120 mg of Compound 1 or a pharmaceutically acceptable salt thereof once a day is selected to provide a substantial reduction in depression.

According to some embodiments of the present invention, the dosing frequency and dose amount per administration of Compound 1 or a pharmaceutically acceptable salt thereof are selected to provide therapeutic effects for the treatment of a mood or affective disorder selected from perimenopause, generalized anxiety disorder, panic disorder, social anxiety disorder, acute stress disorder, post-traumatic stress disorder, specific phobia, and selective mutism.

In some embodiments, the dosing frequency and dose amount per administration of Compound 1 or a pharmaceutically acceptable salt thereof are selected to provide therapeutic effects for the treatment of perimenopause. In some embodiments, the dosing frequency and dose amount per administration of Compound 1 or a pharmaceutically acceptable salt thereof are selected to provide therapeutic effects for the treatment of perimenopausal depression. Methods of diagnosing perimenopausal depression are described in the art, such as set forth in Pauline M. Maki, et al. Guidelines for the Evaluation and Treatment of Perimenopausal Depression: Summary and Recommendations. Journal of Women's Health (DOI: 10.1089/jwh.2018.27099.mensocrec). In some embodiments, the dosing frequency and dose amount per administration of Compound 1 or a pharmaceutically acceptable salt thereof are selected to provide therapeutic effects for the treatment of perimenopause anxiety. In some embodiments, the dosing frequency and dose amount per administration of Compound 1 or a pharmaceutically acceptable salt thereof are selected to provide therapeutic effects for the treatment of perimenopause agitation.

In some embodiments, the dosing frequency and dose amount per administration of Compound 1 or a pharmaceutically acceptable salt thereof are selected to provide therapeutic effects for the treatment of menopause anxiety or postmenopause anxiety.

In some embodiments, the dosing frequency and dose amount per administration of Compound 1 or a pharmaceutically acceptable salt thereof are selected to provide therapeutic effects for the treatment of menopause agitation or postmenopause agitation.

In some embodiments, the dosing frequency and dose amount per administration of Compound 1 or a pharmaceutically acceptable salt thereof are selected to provide therapeutic effects for the acute treatment of depression. In some embodiments, the dosing frequency and dose amount per administration of Compound 1 or a pharmaceutically acceptable salt thereof are selected to provide therapeutic effects for the acute treatment of depression and, after the acute treatment of depression, Compound 1 or a pharmaceutically acceptable salt thereof is no longer administered and a dosing frequency and dose amount of second anti-depressant agent is selected to provide therapeutic effects for the chronic treatment of depression.

In some embodiments, the dosing frequency and dose amount per administration of Compound 1 or a pharmaceutically acceptable salt thereof are selected to provide therapeutic effects for the acute treatment of depression and, after the acute treatment of depression, a dosing frequency and dose amount of Compound 1 or a pharmaceutically acceptable salt thereof is selected to provide therapeutic effects for the chronic treatment of depression. In certain embodiments, the daily dosing of Compound 1 or a pharmaceutically acceptable salt thereof for the acute treatment of depression is greater than the daily dosing of Compound 1 or a pharmaceutically acceptable salt thereof for the chronic treatment of depression.

In some embodiments, the dosing frequency and dose amount per administration of Compound 1 or a pharmaceutically acceptable salt thereof are selected to prevent the recurrence of depression. In some embodiments, the dosing frequency and dose amount per administration of Compound 1 or a pharmaceutically acceptable salt thereof are selected to maintain remission of depression.

In some embodiments, the dosing frequency, dose amount per administration of Compound 1 or a pharmaceutically acceptable salt thereof, and dosing time are selected to provide a therapeutic effect for the treatment of depression, perimenopause, generalized anxiety disorder, panic disorder, social anxiety disorder, acute stress disorder, post-traumatic stress disorder, specific phobia, or selective mutism and to reduce adverse effects associated with administration of Compound 1 or a pharmaceutically acceptable salt thereof. In some embodiments, the methods of the disclosure provide reduced somnolence compared to morning administration of Compound 1 or a pharmaceutically acceptable salt thereof. In some embodiments, the methods of the disclosure provide reduced dizziness compared to morning administration of Compound 1 or a pharmaceutically acceptable salt thereof. In some embodiments, the methods of the disclosure provide reduced lightheadedness, fatigue, euphoria, feeling drunk, feeling abnormal, headache, cognitive disorder, disturbance of attention, memory impairment, slowed cognition, decreased alertness, constipation, or diarrhea compared to morning administration of Compound 1 or a pharmaceutically acceptable salt thereof.

In certain embodiments, Compound 1 or a pharmaceutically acceptable salt thereof is administered on a once-a-day or twice-a-day basis for at least a week, for example, about a week, about 2 weeks, about 3 weeks, about 4 weeks, about 5 weeks, about 6 weeks, about 7 weeks, about 8 weeks, about 9 weeks, about 10 weeks, about 12 weeks, about 18 weeks, about 24 weeks, and about 50 weeks.

In certain embodiments, at least about 5 mg or about 5 mg of Compound 1 or a pharmaceutically acceptable salt thereof is administered on a once-a-day or twice-a-day basis for at least a week. In certain embodiments, at least about 10 mg or about 10 mg of Compound 1 or a pharmaceutically acceptable salt thereof is administered on a once-a-day or twice-a-day basis for at least a week. In certain embodiments, at least about 15 mg or about 15 mg of Compound 1 or a pharmaceutically acceptable salt thereof is administered on a once-a-day or twice-a-day basis for at least a week. In certain embodiments, at least about 20 mg or about 20 mg of Compound 1 or a pharmaceutically acceptable salt thereof is administered on a once-a-day or twice-a-day basis for at least a week. In certain embodiments, at least about 25 mg or about 25 mg of Compound 1 or a pharmaceutically acceptable salt thereof is administered on a once-a-day or twice-a-day basis for at least a week. In certain embodiments, at least about 30 mg or about 30 mg of Compound 1 or a pharmaceutically acceptable salt thereof is administered on a once-a-day or twice-a-day basis for at least a week. In certain embodiments, at least about 35 mg or about 35 mg of Compound 1 or a pharmaceutically acceptable salt thereof is administered on a once-a-day or twice-a-day basis for at least a week. In certain embodiments, at least about 40 mg or about 40 mg of Compound 1 or a pharmaceutically acceptable salt thereof is administered on a once-a-day or twice-a-day basis for at least a week. In certain embodiments, at least about 45 mg or about 45 mg of Compound 1 or a pharmaceutically acceptable salt thereof is administered on a once-a-day or twice-a-day basis for at least a week. In certain embodiments, at least about 50 mg or about 50 mg of Compound 1 or a pharmaceutically acceptable salt thereof is administered on a once-a-day or twice-a-day basis for at least a week. In certain embodiments, at least about 55 mg or about 55 mg of Compound 1 or a pharmaceutically acceptable salt thereof is administered on a once-a-day or twice-a-day basis for at least a week. In certain embodiments, at least about 60 mg or about 60 mg of Compound 1 or a pharmaceutically acceptable salt thereof is administered on a once-a-day or twice-a-day basis for at least a week. In certain embodiments, at least about 65 mg or about 65 mg of Compound 1 or a pharmaceutically acceptable salt thereof is administered on a once-a-day basis for at least a week. In certain embodiments, at least about 70 mg or about 70 mg of Compound 1 or a pharmaceutically acceptable salt thereof is administered on a once-a-day basis for at least a week. In certain embodiments, at least about 75 mg or about 75 mg of Compound 1 or a pharmaceutically acceptable salt thereof is administered on a once-a-day basis for at least a week. In certain embodiments, at least about 80 mg or about 80 mg of Compound 1 or a pharmaceutically acceptable salt thereof is administered on a once-a-day basis for at least a week. In certain embodiments, at least about 85 mg or about 85 mg of Compound 1 or a pharmaceutically acceptable salt thereof is administered on a once-a-day basis for at least a week. In certain embodiments, at least about 90 mg or about 90 mg of Compound 1 or a pharmaceutically acceptable salt thereof is administered on a once-a-day basis for at least a week. In certain embodiments, at least about 95 mg or about 95 mg of Compound 1 or a pharmaceutically acceptable salt thereof is administered on a once-a-day basis for at least a week. In certain embodiments, at least about 100 mg or about 100 mg of Compound 1 or a pharmaceutically acceptable salt thereof is administered on a once-a-day basis for at least a week. In certain embodiments, at least about 105 mg or about 105 mg of Compound 1 or a pharmaceutically acceptable salt thereof is administered on a once-a-day basis for at least a week. In certain embodiments, at least about 110 mg or about 0.110 mg of Compound 1 or a pharmaceutically acceptable salt thereof is administered on a once-a-day basis for at least a week. In certain embodiments, at least about 115 mg or about 115 mg of Compound 1 or a pharmaceutically acceptable salt thereof is administered on a once-a-day basis for at least a week. In certain embodiments, at least about 120 mg or about 120 mg of Compound 1 or a pharmaceutically acceptable salt thereof is administered on a once-a-day basis for at least a week.

In some embodiments, the method of treating depression further includes a step of titrating the dose of Compound 1 or a pharmaceutically acceptable salt thereof until a maintenance dose is achieved in the patient. In some embodiments, the titration is conducted for at least about one week until a maintenance dose is achieved in the patient. In one embodiment, the titration is conducted for about 2 weeks until a maintenance dose is achieved in the patient. In some embodiments, the titration is conducted for about 7 days to about 30 days until a maintenance dose is achieved in the patient. In some embodiments, the titration is conducted for about 12 days to about 20 days until a maintenance dose is achieved in the patient. In some embodiments, during the titration step, a constant daily dose of Compound 1 or a pharmaceutically acceptable salt thereof is provided. In further embodiments, the constant daily dose of Compound 1 or a pharmaceutically acceptable salt thereof is provided for at least two weeks.

The daily dose can be titrated in one or more steps. The daily dosage can be titrated by increasing a single daily dosage or each dose of a twice-daily dosing regimen. The amount a dosage is stepped, where there are multiple titration steps, can be the same or can be different.

In some embodiments, the titration is initiated with from about 10 mg to about 100 mg of Compound 1 or a pharmaceutically acceptable salt thereof, including about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, and about 100 mg, including all ranges there between once or twice daily. In some embodiments, the titration is initiated with about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, and about 100 mg of Compound 1 or a pharmaceutically acceptable salt thereof once or twice daily. In certain embodiments, doses can be adjusted in 5-30 mg increments every 1 to 4 days. In certain embodiments, doses can be adjusted in 5-30 mg increments every week. In some embodiments, the titration is conducted for at least about one week, about 2 weeks, about 3 weeks, about 4 weeks, about 5 weeks, about 6 weeks, about 7 weeks, about 8 weeks, about 9 weeks, or about 10 weeks prior to the maintenance dose.

In certain embodiments, ascending doses of Compound 1 or a pharmaceutically acceptable salt thereof are administered during the titration until a maintenance dose is achieved in the patient. In certain embodiments, ascending doses of the Compound 1 or a pharmaceutically acceptable salt thereof are administered during the titration until an effective amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg is achieved in the patient.

In certain embodiments, patients are initially administered about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg of Compound 1 or a pharmaceutically acceptable salt once or twice a day and are titrated to a maintenance dose of about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg once or twice a day. In certain embodiments, patients are initially administered from about 15 mg to about 100 mg of Compound 1 or a pharmaceutically acceptable salt, including about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, and about 100 mg, including all ranges there between once or twice a day and are titrated to a maintenance dose of from about 20 mg to about 120 mg, including about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg including all ranges therebetween, once or twice a day.

In some embodiments, the present disclosure provides a method of treating depression that includes the steps of: (a) administering an initial daily dose of Compound 1 or a pharmaceutically acceptable salt thereof for at least one week and (b) administering a maintenance daily dose for at least one week. In certain embodiments, the initial daily dose is greater than the maintenance daily dose. In certain embodiments, the initial daily dose is less than the maintenance daily dose. In certain embodiments, the initial daily dose is administered for two weeks, and the maintenance daily dose is administered for at least one month.

In some embodiments, the present disclosure provides a method of treating depression that includes the steps of:
(a) administering a loading dose of Compound 1 or a pharmaceutically acceptable salt thereof and
(b) administering a maintenance dose of Compound 1 or a pharmaceutically acceptable salt thereof.

In some embodiments, the loading dose is administered for about 1 day to about 14 days, including about 1 day, about 2 days, about 3 days, about 4 days, about 5 days, about 6 days, about 7 days, about 8 days, about 9 days, about 10 days, about 11 days, about 12 days, about 13 days and about 14 days, including all ranges therebetween. In some embodiments, the loading dose is administered for about 1 day, about 2 days, about 3 days, about 4 days, about 5 days, about 6 days, about 7 days, about 8 days, about 9 days, about 10 days, about 11 days, about 12 days, about 13 days or about 14 days.

In some embodiments, the methods comprise administering a loading dose of from about 5 mg to about 120 mg, including about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, and about 120 mg, including all ranges therebetween. In some embodiments, the methods comprise administering a loading dose of about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg.

In some embodiments, the maintenance dose is administered for from about 1 month to about 36 months, including about 1 month, about 2 months, about 3 months, about 4 months, about 5 months, about 6 months, about 7 months, about 8 months, about 9 months, about 10 months, about 11 months, about 12 months, about 18 months, about 24 months, about 30 months, or about 36 months, including all ranges there between. In some embodiments, the maintenance dose is administered for about 1 month, about 2 months, about 3 months, about 4 months, about 5 months, about 6 months, about 7 months, about 8 months, about 9 months, about 10 months, about 11 months, about 12 months, about 18 months, about 24 months, about 30 months, or about 36 months.

In some embodiments, the methods comprise administering a maintenance dose of from about 5 mg to about 120 mg, including about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, and about 120 mg, including all ranges therebetween, once or twice a day. In some embodiments, the methods comprise administering a maintenance dose of from about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg, once or twice a day.

In some embodiments, the loading dose administration methods further comprise a cessation period after administration of the loading dose and prior to administration of the maintenance dose.

In some embodiments, the cessation period is about one day, about two days, about three days, about four days, about five days, about six days, or about seven days. In some embodiments, the cessation period is from about one day to about seven days, including about one day, about two days, about three days, about four days, about five days, about six days, and about seven days, including all ranges there between.

In some embodiments, the cessation period is about one week, about two weeks, about three weeks, about four weeks, about five weeks, about six weeks, about seven weeks, or about eight weeks. In some embodiments, the cessation period is from about one week to about eight weeks, including about one week, about two weeks, about three weeks, about four weeks, about five weeks, about six weeks, about seven weeks, and about eight weeks, including all ranges there between.

In some embodiments, the methods of the present disclosure comprise continuous administration of Compound 1 or a pharmaceutically acceptable salt thereof for a specified interval (for example, one week) followed by a cessation period wherein the patient is not administered Compound 1 or a pharmaceutically acceptable salt thereof. In some embodiments, the methods of the present disclosure comprise continuous administration of Compound 1 or a pharmaceutically acceptable salt thereof for from about one week to about eight weeks, including about one week, about two weeks, about three weeks, about four weeks, about five weeks, about six weeks, about seven weeks, and about eight weeks, including all ranges there between. In some embodiments, the methods of the present disclosure comprise continuous administration of Compound 1 or a pharmaceutically acceptable salt thereof for about one week, about two weeks, about three weeks, about four weeks, about five weeks, about six weeks, about seven weeks, or about eight weeks.

In some embodiments, the methods of the present disclosure comprise continuous administration of Compound 1 or a pharmaceutically acceptable salt thereof for from about one month to about 36 months, including about one month, about two months, about three months, about four months, about five months, about six months, about seven months, about eight months, about 9 months, about 10 months, about 11 months, about 12 months, about 18 months, about 24 months, about 30 months, and about 36 months, including all ranges there between. In some embodiments, the methods of the present disclosure comprise continuous administration of Compound 1 or a pharmaceutically acceptable salt thereof for about one month, about two months, about three months, about four months, about five months, about six months, about seven months, about eight months, about nine months, about ten months, about eleven months, about twelve months, about 18 months, about 24 months, about 30 months, or about 36 months.

In some embodiments, the methods of the present disclosure comprise intermittent administration of Compound 1 or a pharmaceutically acceptable salt thereof. As used herein, intermittent administration means cycling a patient in need thereof on and off treatment with Compound 1 or a pharmaceutically acceptable salt thereof for specified time intervals.

In some embodiments, intermittent administration comprises:
(a) administering Compound 1 or a pharmaceutically acceptable salt thereof for a first administration period;
(b) after the first administration period (a), not administering Compound 1 or a pharmaceutically acceptable salt thereof for a cessation period;
(c) after the cessation period (b), administering Compound 1 or a pharmaceutically acceptable salt thereof for a second administration period.

In some embodiments, the intermittent administration further comprises one or more additional cessation periods (for example, a second cessation period) and/or administration periods (for example, a third administration period). In such embodiments, the present disclosure contemplates embodiments wherein the additional cessation and/or administration periods have the durations described herein for the first administration period and the cessation period.

In some embodiments, two or more of the periods (a), (b) and (c) are the same (for example, the first administration period, cessation period and second administration period are each one week). In some embodiments, the periods (a) and (b) (for example, one week) are the same and the period (c) is different (for example, two weeks). In some embodiments, the periods (a) and (c) are the same and the period (b) is different. In some embodiments, the periods (b) and (c) are the same and the period (a) is different. In some embodiments, the periods (a), (b) and (c) are each different (for example, the first administration period is one week, the cessation period is two weeks, and the second administration period is three weeks).

In some embodiments, the first administration period is about one week, about two weeks, about three weeks, about four weeks, about five weeks, about six weeks, about seven weeks, or about eight weeks. In some embodiments, the first administration period is from about one week to about eight weeks, including about one week, about two weeks, about three weeks, about four weeks, about five weeks, about six weeks, about seven weeks, and about eight weeks, including all ranges there between.

In some embodiments, the cessation period is about one week, about two weeks, about three weeks, about four weeks, about five weeks, about six weeks, about seven weeks, or about eight weeks. In some embodiments, the cessation period is from about one week to about eight weeks, including about one week, about two weeks, about three weeks, about four weeks, about five weeks, about six weeks, about seven weeks, and about eight weeks, including all ranges there between.

In some embodiments, the second administration period is about one week, about two weeks, about three weeks, about four weeks, about five weeks, about six weeks, about seven weeks, or about eight weeks. In some embodiments, the second administration period is from about one week to about eight weeks, including about one week, about two weeks, about three weeks, about four weeks, about five weeks, about six weeks, about seven weeks, or about eight weeks, including all ranges there between.

In some embodiments, the first administration period, cessation period and second administration period are one week. In some embodiments, the first administration period, cessation period and second administration period are two weeks. In some embodiments, the first administration period, cessation period and second administration period are three weeks.

In some embodiments, the first administration period, cessation period and second administration period are four weeks. In some embodiments, the first administration period, cessation period and second administration period are five weeks. In some embodiments, the first administration period, cessation period and second administration period are six weeks. In some embodiments, the first administration period, cessation period and second administration period are seven weeks. In some embodiments, the first administration period, cessation period and second administration period are eight weeks.

In some embodiments, the first administration period is about one week; the cessation period is about three weeks; and the second administration period is about one week.

In some embodiments, the first administration period is about two weeks; the first cessation period is about two weeks; the second administration period is about one week; the second cessation period is about one week; and the third administration period is about one week.

In some embodiments, the intermittent administration period (including the administration and cessation periods) is about one month, about two months, about three months, about four months, about five months, about six months, about seven months, about eight months, about nine months, about ten months, about eleven months, about twelve months, about 18 months, about 24 months, about 30 months or about 36 months. In some embodiments, the intermittent administration period is from about one month to about 36 months, including about two months, about three months, about four months, about five months, about six months, about seven months, about eight months, about nine months, about ten months, about eleven months, about twelve months, about 18 months, about 24 months, about 30 months and about 36 months, including all ranges there between.

In some embodiments of the present disclosure, Compound 1 or a pharmaceutically acceptable salt thereof is administered to a patient with food. In some embodiments, Compound 1 or a pharmaceutically acceptable salt thereof is administered to a patient about 5 minutes, about 10 minutes, about 15 minutes, about 30 minutes, about 45 minutes, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours or about 8 hours after food is ingested.

In some embodiments, the food ingested is a high fat and high calorie food. In some embodiments, the caloric content of the high fat and high calorie food is at least about 700 kilocalories (kcal), and at least about 40 percent of the caloric content of the food is from fat. For example, the fat can contribute to about 50 percent of the caloric content of the high fat and high calorie food. In some embodiments, the caloric content of the high fat and high calorie food is about 900 kilocalories.

In some embodiments, the food ingested is a medium fat and medium calorie food. In some embodiments, the caloric content of the medium fat and medium calorie food is about 300 kcal to about 700 kcal, and between about 20 percent to about 40 percent of the caloric content of the food is from fat. In some embodiments, the caloric content of the medium fat and medium calorie food is about 400 kcal.

In some embodiments, the food ingested is a low fat and low-calorie food. In some embodiments, the caloric content of the low fat and low-calorie food is between about 100 kcal to about 300 kcal, and the fat content is approximately 3 grams or less, or about 20 percent or less of the caloric content of the food are from fat. In some embodiments, the caloric content of the low fat and low-calorie food is about 100 kilocalories.

In some embodiments of the present disclosure, Compound 1 or a pharmaceutically acceptable salt thereof is administered to a patient after a fasting period. In some embodiments, Compound 1 or a pharmaceutically acceptable salt thereof is administered to a patient after a fasting period of about 1 hour, about 2 hours, about 3 hours, about 4 hours, about 5 hours, about 6 hours, about 7 hours, about 8 hours, about 9 hours, about 10 hours, about 11 hours, or about 12 hours. In some embodiments, Compound 1 or a pharmaceutically acceptable salt thereof is administered on an empty stomach.

In some embodiments of the present disclosure, Compound 1 or a pharmaceutically acceptable salt thereof is administered to a patient without regard to meals. In some embodiments, Compound 1 or a pharmaceutically acceptable salt thereof is administered to a patient with or without a meal.

In some embodiments, Compound 1 or a pharmaceutically acceptable salt thereof is administered to a patient in the evening. In some embodiments, administration in the evening occurs at bedtime. In some embodiments, Compound 1 or a pharmaceutically acceptable salt thereof is administered to a patient at bedtime. In some embodiments, Compound 1 or a pharmaceutically acceptable salt thereof is administered to a patient prior to bedtime. A patient's bedtime may be at any time of the day, for example, at about 12:00 a.m., at about 12:30 a.m., at about 1:00 a.m., at about 1:30 a.m., at about 2:00 a.m., at about 2:30 a.m., at about 3:00 a.m., at about 3:30 a.m., at about 4:00 a.m., at about 4:30 a.m., at about 5:00 a.m., at about 5:30 a.m., at about 6:00 a.m., at about 6:30 a.m., at about 7:00 a.m., at about 7:30 a.m., at about 8:00 a.m., at about 8:30 a.m., at about 9:00 a.m., at about 9:30 a.m., at about 10:00 a.m., at about 10:30 a.m., at about 11:00 a.m., at about 11:30 a.m., at about 12:00 p.m., at about 12:30 p.m., at about 1:00 p.m., at about 1:30 p.m., at about 2:00 p.m., at about 2:30 p.m., at about 3:00 p.m., at about 3:30 p.m., at about 4:00 p.m., at about 4:30 p.m., at about 5:00 p.m., at about 5:30 p.m., at about 6:00 p.m., at about 6:30 p.m., at about 7:00 p.m., at about 7:30 p.m., at about 8:00 p.m., at about 8:30 p.m., at about 9:00 p.m., at about 9:30 p.m., at about 10:00 p.m., at about 10:30 p.m., at about 11:00 p.m., at about 11:30 p.m.

In some embodiments, the administration in the evening occurs prior to bedtime. In some embodiments, administration in the evening occurs within about three hours of bedtime, for example, about three hours prior to bedtime, about 2.5 hours prior to bedtime, about 2 hours prior to bedtime, about 1.5 hours prior to bedtime, about 1 hour prior to bedtime, about 30 minutes prior to bedtime, about 20 minutes prior to bedtime, about 15 minutes prior to bedtime, about 10 minutes prior to bedtime, or about 5 minutes prior to bedtime. In some embodiments, administration in the evening occurs within about two hours prior to bedtime.

In some embodiments, administration in the evening occurs between about 5 p.m. and about 12 a.m., for example, at about 5 p.m., at about 5:30 p.m., at about 6:00 p.m., at about 6:30 p.m., at about 7:00 p.m., at about 7:30 p.m., at about 8:00 p.m., at about 8:30 p.m., at about 9:00 p.m., at about 9:30 p.m., at about 10:00 p.m., at about 10:30 p.m., at about 11:00 p.m., at about 11:30 p.m., or at about 12:00 a.m.

In some embodiments, administration in the evening occurs after 8 p.m., for example, at about 8 p.m., at about 8:30 p.m., at about 9:00 p.m., at about 9:30 p.m., at about 10:00 p.m., at about 10:30 p.m., at about 11:00 p.m., at about 11:30 p.m., at about 12:00 a.m., at about 12:30 a.m., at about 1:00 a.m., at about 1:30 a.m., or about 2:00 a.m.

In some embodiments, administration in the evening occurs between about 5 p.m. and bedtime.

In some embodiments, administration in the evening occurs with the evening meal. In some embodiments, administration in the evening occurs within about six hours of the evening meal. In some embodiments, administration in the evening occurs within about five hours of the evening meal. In some embodiments, administration in the evening occurs within about four hours of the evening meal. In some embodiments, administration in the evening occurs within about three hours of the evening meal. In some embodiments, administration in the evening occurs within about two hours of the evening meal. In some embodiments, administration in the evening occurs within about one hour of the evening meal. In some embodiments, administration in the evening occurs within about 30 minutes of the evening meal. In some embodiments, administration in the evening occurs two hours after the evening meal. In some embodiments, administration in the evening occurs four hours after the evening meal. In some embodiments, administration in the evening occurs after the evening meal. In some embodiments, the evening meal is dinner. In some embodiments, administration in the evening occurs two hours after dinner. In some embodiments, administration in the evening occurs four hours after dinner. In some embodiments, administration in the evening occurs after dinner.

In some embodiments, Compound 1 or a pharmaceutically acceptable salt thereof is administered to a patient in the morning. In some embodiments, a morning dose is administered when a patient wakes up. A patient may wake up at any time of the day, for example, at about 12:00 a.m., at about 12:30 a.m., at about 1:00 a.m., at about 1:30 a.m., at about 2:00 a.m., at about 2:30 a.m., at about 3:00 a.m., at about 3:30 a.m., at about 4:00 a.m., at about 4:30 a.m., at about 5:00 a.m., at about 5:30 a.m., at about 6:00 a.m., at about 6:30 a.m., at about 7:00 a.m., at about 7:30 a.m., at about 8:00 a.m., at about 8:30 a.m., at about 9:00 a.m., at about 9:30 a.m., at about 10:00 a.m., at about 10:30 a.m., at about 11:00 a.m., at about 11:30 a.m., at about 12:00 p.m., at about 12:30 p.m., at about 1:00 p.m., at about 1:30 p.m., at about 2:00 p.m., at about 2:30 p.m., at about 3:00 p.m., at about 3:30 p.m., at about 4:00 p.m., at about 4:30 p.m., at about 5:00 p.m., at about 5:30 p.m., at about 6:00 p.m., at about 6:30 p.m., at about 7:00 p.m., at about 7:30 p.m., at about 8:00 p.m., at about 8:30 p.m., at about 9:00 p.m., at about 9:30 p.m., at about 10:00 p.m., at about 10:30 p.m., at about 11:00 p.m., at about 11:30 p.m. In some embodiments, administration in the morning occurs within about three hours after a patient wakes up, for example, about three hours after a patient wakes up, about 2.5 hours after a patient wakes up, about 2 hours after a patient wakes up, about 1.5 hours after a patient wakes up, about 1 hour after a patient wakes up, about 30 minutes after a patient wakes up, about 20 minutes after a patient wakes up, about 15 minutes after a patient wakes up, about 10 minutes after a patient wakes up, or about 5 minutes after a patient wakes up. In some embodiments, administration in the morning occurs within about two hours after a patient wakes up. In some embodiments, Compound 1 or a pharmaceutically acceptable salt thereof is administered in the morning after a period of fasting.

In some embodiments, administration in the morning occurs between about 5 a.m. and about 12 p.m., for example, at about 5 a.m., at about 5:30 a.m., at about 6:00 a.m., at about 6:30 a.m., at about 7:00 a.m., at about 7:30 a.m., at about 8:00 a.m., at about 8:30 a.m., at about 9:00 a.m., at about 9:30 a.m., at about 10:00 a.m., at about 10:30 a.m., at about 11:00 a.m., at about 11:30 a.m., or at about 12:00 p.m.

In some embodiments, administration in the morning occurs at or after 5:00 a.m., for example, at about 5:00 a.m., at about 5:30 a.m., at about 6:00 a.m., at about 6:30 a.m., at about 7:00 a.m., at about 7:30 a.m., at about 8:00 a.m., at about 8:30 a.m., at about 9:00 a.m., at about 9:30 a.m., at about 10:00 a.m., at about 10:30 a.m., at about 11:00 a.m., at about 11:30 a.m., or at about 12:00 p.m.

In some embodiments, administration in the morning occurs between about 5 a.m. and 12:00 p.m., for example, at about 5:00 a.m., at about 5:30 a.m., at about 6:00 a.m., at about 6:30 a.m., at about 7:00 a.m., at about 7:30 a.m., at about 8:00 a.m., at about 8:30 a.m., at about 9:00 a.m., at about 9:30 a.m., at about 10:00 a.m., at about 10:30 a.m., at about 11:00 a.m., at about 11:30 a.m., or at about 12:00 p.m.

In some embodiments, administration in the morning occurs with the morning meal. In some embodiments, administration in the morning occurs within about six hours of the morning meal. In some embodiments, administration in the morning occurs within about five hours of the morning meal. In some embodiments, administration in the morning occurs within about four hours of the morning meal. In some embodiments, administration in the morning occurs within about three hours of the morning meal. In some embodiments, administration in the morning occurs within about two hours of the morning meal. In some embodiments, administration in the morning occurs within about one hour of the morning meal. In some embodiments, administration in the morning occurs within about 30 minutes of the morning meal.

In some embodiments, administration in the morning occurs between about eight hours and about 16 hours before administration of an evening dose. In some embodiments, administration in the morning occurs between about ten hours and about 14 hours before administration of an evening dose. In some embodiments, administration in the morning occurs about eight hours before administration of an evening dose. In some embodiments, administration in the morning occurs about nine hours before administration of an evening dose.

In some embodiments, administration in the morning occurs about ten hours before administration of an evening dose. In some embodiments, administration in the morning occurs about 11 hours before administration of an evening dose. In some embodiments, administration in the morning occurs about 12 hours before administration of an evening dose. In some embodiments, administration in the morning occurs about 13 hours before administration of an evening dose. In some embodiments, administration in the morning occurs about 14 hours before administration of an evening dose. In some embodiments, administration in the morning occurs about 15 hours before administration of an evening dose. In some embodiments, administration in the morning occurs about 16 hours before administration of an evening dose.

In some embodiments, when the dose of Compound 1 or a pharmaceutically acceptable salt thereof is administered during the day for treating tremor, the dose of Compound 1 or an equivalent dose of a pharmaceutically acceptable salt thereof, is no more than about 40 mg, more particularly in the range of about 5 mg to about 40 mg (including about 5 mg, about 10 mg, about 15 mg, about 20 mg, about 25 mg, about 30 mg, about 35 mg, or about 40 mg).

In some embodiments, the methods of the present disclosure comprise controlling the gastrointestinal pH of the patient prior to, concurrently with or after administration of Compound 1 or a pharmaceutically acceptable salt thereof. In some embodiments, the gastrointestinal pH of the patient is controlled prior to administration of Compound 1 or a pharmaceutically acceptable salt thereof. In some embodiments, the gastrointestinal pH of the patient is controlled after administration of Compound 1 or a pharmaceutically acceptable salt thereof.

In some embodiments, the pH is controlled by administering a drug, food or liquid to a patient that decreases gastrointestinal pH prior to, concurrently with or after administration of Compound 1 or a pharmaceutically acceptable salt thereof. In some embodiments, the liquid is an acidic beverage (such as a carbonated beverage).

In some embodiments, the pH is controlled by the patient avoiding a drug, food or beverage that increases gastrointestinal pH prior to, concurrently with or after administration of Compound 1 or a pharmaceutically acceptable salt thereof. In some embodiments, the drug that increases gastrointestinal pH is a proton pump inhibitor or an orally administered antacid.

In some embodiments, the initial daily dosing frequency and dose amount per administration of Compound 1 or a pharmaceutically acceptable salt thereof are selected to provide therapeutic effects for the acute treatment of depression, and the maintenance daily dosing frequency and dose amount per administration of Compound 1 or a pharmaceutically acceptable salt are selected to provide therapeutic effects for the chronic treatment of depression.

In some embodiments, the initial daily dosing frequency and dose amount per administration of Compound 1 or a pharmaceutically acceptable salt thereof are selected to provide therapeutic effects for the acute treatment of depression, and the maintenance daily dosing frequency and dose amount per administration of Compound 1 or a pharmaceutically acceptable salt thereof are selected to maintain remission of depression.

In some embodiments, the initial daily dosing frequency and dose amount per administration of Compound 1 or a pharmaceutically acceptable salt thereof are selected to provide therapeutic effects for the acute treatment of depression, and the maintenance the daily dosing frequency and dose amount per administration of Compound 1 or a pharmaceutically acceptable salt thereof are selected to prevent recurrence of depression.

In some embodiments, the initial daily dosing frequency and dose amount per administration of Compound 1 or a pharmaceutically acceptable salt thereof are selected to provide therapeutic effects for the treatment of tremor (including essential tremor), and the maintenance the daily dosing frequency and dose amount per administration of Compound 1 or a pharmaceutically acceptable salt thereof are selected to prevent or reduce the recurrence or frequency of tremor.

In some embodiments, the initial daily dosing frequency and dose amount per administration of Compound 1 or a pharmaceutically acceptable salt thereof are selected to provide therapeutic effects for the treatment of fibromyalgia and related musculoskeletal conditions, and the maintenance the daily dosing frequency and dose amount per administration of Compound 1 or a pharmaceutically acceptable salt thereof are selected to prevent recurrence of fibromyalgia and related musculoskeletal conditions.

In certain embodiments, the initial daily dose is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg or about 120 mg, and the maintenance daily dose is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, or about 115 mg, provided the initial daily dose is greater than the maintenance daily dose.

In some embodiments, the total daily dose of Compound 1 or a pharmaceutically acceptable salt thereof is at least about 5 mg a day for the treatment of tremor. In some embodiments, the total daily dose of Compound 1 or a pharmaceutically acceptable salt thereof is at least about 10 mg a day for the treatment of tremor. In some embodiments, the total daily dose of Compound 1 or a pharmaceutically acceptable salt thereof is at least about 15 mg a day for the treatment of tremor. In some embodiments, the total daily dose of Compound 1 or a pharmaceutically acceptable salt thereof is at least about 20 mg a day for the treatment of tremor. In some embodiments, the total daily dose of Compound 1 or a pharmaceutically acceptable salt thereof is at least about 25 mg a day for the treatment of tremor. In some embodiments, the total daily dose of Compound 1 or a pharmaceutically acceptable salt thereof is at least about 30 mg a day for the treatment of tremor. In some embodiments, the total daily dose of Compound 1 or a pharmaceutically acceptable salt thereof is at least about 35 mg a day for the treatment of tremor. In some embodiments, the total daily dose of Compound 1 or a pharmaceutically acceptable salt thereof is at least about 40 mg a day for the treatment of tremor. In some embodiments, the total daily dose of Compound 1 or a pharmaceutically acceptable salt thereof is at least about 45 mg a day for the treatment of tremor. In some embodiments, the total daily dose of Compound 1 or a pharmaceutically acceptable salt thereof is at least about 50 mg a day for the treatment of tremor. In some embodiments, the total daily dose of Compound 1 or a pharmaceutically acceptable salt thereof is at least about 55 mg a day for the treatment of tremor. In some embodiments, the total daily dose of Compound 1 or a pharmaceutically acceptable salt thereof is at least about 60 mg a day for the treatment of tremor. In some embodiments, the total daily dose of Compound 1 or a pharmaceutically acceptable salt thereof is at least about 65 mg a day for the treatment of tremor. In some embodiments, the total daily dose of Compound 1 or a pharmaceutically acceptable salt thereof is at least about 70 mg a day for the treatment of tremor. In some embodiments, the total daily dose of Compound 1 or a pharmaceutically acceptable salt thereof is at least about 75 mg a day for the treatment of tremor. In some embodiments, the total daily dose of Compound 1 or a pharmaceutically acceptable salt thereof is at least about 80 mg a day for the treatment of tremor. In some embodiments, the total daily dose of Compound 1 or a pharmaceutically acceptable salt thereof is at least about 85 mg a day for the treatment of tremor. In some embodiments, the total daily dose of Compound 1 or a pharmaceutically acceptable salt thereof is at least about 90 mg a day for the treatment of tremor. In some embodiments, the total daily dose of Compound 1 or a pharmaceutically acceptable salt thereof is at least about 95 mg a day for the treatment of tremor. In some embodiments, the total daily dose of Compound 1 or a pharmaceutically acceptable salt thereof is at least about 100 mg a day for the treatment of tremor. In some embodiments, the total daily dose of Compound 1 or a pharmaceutically acceptable salt thereof is at least about 105 mg a day for the treatment of tremor. In some embodiments, the total daily dose of Compound 1 or a pharmaceutically acceptable salt thereof is at least about 110 mg a day for the treatment of tremor. In some embodiments, the total daily dose of Compound 1 or a pharmaceutically acceptable salt thereof is at least about 115 mg a day for the treatment of tremor. In some embodiments, the total daily dose of Compound 1 or a pharmaceutically acceptable salt thereof is at least about 120 mg a day for the treatment of tremor.

In some embodiments, the total daily dose of Compound 1 or a pharmaceutically acceptable salt thereof is about 5 mg a day for the treatment of tremor. In some embodiments, the total daily dose of Compound 1 or a pharmaceutically acceptable salt thereof is about 10 mg a day for the treatment of tremor. In some embodiments, the total daily dose of Compound 1 or a pharmaceutically acceptable salt thereof is about 15 mg a day for the treatment of tremor. In some embodiments, the total daily dose of Compound 1 or a pharmaceutically acceptable salt thereof is about 20 mg a day for the treatment of tremor. In some embodiments, the total daily dose of Compound 1 or a pharmaceutically acceptable salt thereof is about 25 mg a day for the treatment of tremor. In some embodiments, the total daily dose of Compound 1 or a pharmaceutically acceptable salt thereof is about 30 mg a day for the treatment of tremor. In some embodiments, the total daily dose of Compound 1 or a pharmaceutically acceptable salt thereof is about 35 mg a day for the treatment of tremor. In some embodiments, the total daily dose of Compound 1 or a pharmaceutically acceptable salt thereof is about 40 mg a day for the treatment of tremor. In some embodiments, the total daily dose of Compound 1 or a pharmaceutically acceptable salt thereof is about 45 mg a day for the treatment of tremor. In some embodiments, the total daily dose of Compound 1 or a pharmaceutically acceptable salt thereof is about 50 mg a day for the treatment of tremor. In some embodiments, the total daily dose of Compound 1 or a pharmaceutically acceptable salt thereof is about 55 mg a day for the treatment of tremor. In some embodiments, the total daily dose of Compound 1 or a pharmaceutically acceptable salt thereof is about 60 mg a day for the treatment of tremor. In some embodiments, the total daily dose of Compound 1 or a pharmaceutically acceptable salt thereof is about 65 mg a day for the treatment of tremor. In some embodiments, the total daily dose of Compound 1 or a pharmaceutically acceptable salt thereof is about 70 mg a day for the treatment of tremor. In some embodiments, the total daily dose of Compound 1 or a pharmaceutically acceptable salt thereof is about 75 mg a day for the treatment of tremor. In some embodiments, the total daily dose of Compound 1 or a pharmaceutically acceptable salt thereof is about 80 mg a day for the treatment of tremor. In some embodiments, the total daily dose of Compound 1 or a pharmaceutically acceptable salt thereof is about 85 mg a day for the treatment of tremor. In some embodiments, the total daily dose of Compound 1 or a pharmaceutically acceptable salt thereof is about 90 mg a day for the treatment of tremor. In some embodiments, the total daily dose of Compound 1 or a pharmaceutically acceptable salt thereof is about 95 mg a day for the treatment of tremor. In some embodiments, the total daily dose of Compound 1 or a pharmaceutically acceptable salt thereof is about 100 mg a day for the treatment of tremor. In some embodiments, the total daily dose of Compound 1 or a pharmaceutically acceptable salt thereof is about 105 mg a day for the treatment of tremor. In some embodiments, the total daily dose of Compound 1 or a pharmaceutically acceptable salt thereof is about 110 mg a day for the treatment of tremor. In some embodiments, the total daily dose of Compound 1 or a pharmaceutically acceptable salt thereof is about 115 mg a day for the treatment of tremor. In some embodiments, the total daily dose of Compound 1 or a pharmaceutically acceptable salt thereof is about 120 mg a day for the treatment of tremor.

In some embodiments, about 5 mg of Compound 1 or a pharmaceutically acceptable salt thereof once a day is selected to provide a substantial reduction in tremor. In some embodiments, about 5 mg of Compound 1 or a pharmaceutically acceptable salt thereof twice a day is selected to provide a substantial reduction in tremor. In some embodiments, about 10 mg of Compound 1 or a pharmaceutically acceptable salt thereof once a day is selected to provide a substantial reduction in tremor. In some embodiments, about 10 mg of Compound 1 or a pharmaceutically acceptable salt thereof twice a day is selected to provide a substantial reduction in tremor. In some embodiments, about 15 mg of Compound 1 or a pharmaceutically acceptable salt thereof once a day is selected to provide a substantial reduction in tremor. In some embodiments, about 15 mg of Compound 1 or a pharmaceutically acceptable salt thereof twice a day is selected to provide a substantial reduction in tremor. In some embodiments, about 20 mg of Compound 1 or a pharmaceutically acceptable salt thereof once a day is selected to provide a substantial reduction in tremor. In some embodiments, about 20 mg of Compound 1 or a pharmaceutically acceptable salt thereof twice a day is selected to provide a substantial reduction in tremor. In some embodiments, about 25 mg of Compound 1 or a pharmaceutically acceptable salt thereof once a day is selected to provide a substantial reduction in tremor. In some embodiments, about 25 mg of Compound 1 or a pharmaceutically acceptable salt thereof twice a day is selected to provide a substantial reduction in tremor. In some embodiments, about 30 mg of Compound 1 or a pharmaceutically acceptable salt thereof once a day is selected to provide a substantial reduction in tremor. In some embodiments, about 30 mg of Compound 1 or a pharmaceutically acceptable salt thereof twice a day is selected to provide a substantial reduction in tremor. In some embodiments, about 30 mg of Compound 1 or a pharmaceutically acceptable salt thereof once a day is selected to provide a substantial reduction in tremor. In some embodiments, about 30 mg of Compound 1 or a pharmaceutically acceptable salt thereof twice a day is selected to provide a substantial reduction in tremor. In some embodiments, about 35 mg of Compound 1 or a pharmaceutically acceptable salt thereof once a day is selected to provide a substantial reduction in tremor. In some embodiments, about 35 mg of Compound 1 or a pharmaceutically acceptable salt thereof twice a day is selected to provide a substantial reduction in tremor. In some embodiments, about 40 mg of Compound 1 or a pharmaceutically acceptable salt thereof once a day is selected to provide a substantial reduction in tremor. In some embodiments, about 40 mg of Compound 1 or a pharmaceutically acceptable salt thereof twice a day is selected to provide a substantial reduction in tremor. In some embodiments, about 45 mg of Compound 1 or a pharmaceutically acceptable salt thereof once a day is selected to provide a substantial reduction in tremor. In some embodiments, about 45 mg of Compound 1 or a pharmaceutically acceptable salt thereof twice a day is selected to provide a substantial reduction in tremor. In some embodiments, about 50 mg of Compound 1 or a pharmaceutically acceptable salt thereof once a day is selected to provide a substantial reduction in tremor. In some embodiments, about 50 mg of Compound 1 or a pharmaceutically acceptable salt thereof twice a day is selected to provide a substantial reduction in tremor. In some embodiments, about 55 mg of Compound 1 or a pharmaceutically acceptable salt thereof once a day is selected to provide a substantial reduction in tremor. In some embodiments, about 55 mg of Compound 1 or a pharmaceutically acceptable salt thereof twice a day is selected to provide a substantial reduction in tremor. In some embodiments, about 60 mg of Compound 1 or a pharmaceutically acceptable salt thereof once a day is selected to provide a substantial reduction in tremor. In some embodiments, about 60 mg of Compound 1 or a pharmaceutically acceptable salt thereof twice a day is selected to provide a substantial reduction in tremor. In some embodiments, about 65 mg of Compound 1 or a pharmaceutically acceptable salt thereof once a day is selected to provide a substantial reduction in tremor. In some embodiments, about 70 mg of Compound 1 or a pharmaceutically acceptable salt thereof once a day is selected to provide a substantial reduction in tremor. In some embodiments, about 75 mg of Compound 1 or a pharmaceutically acceptable salt thereof once a day is selected to provide a substantial reduction in tremor. In some embodiments, about 80 mg of Compound 1 or a pharmaceutically acceptable salt thereof once a day is selected to provide a substantial reduction in tremor. In some embodiments, about 85 mg of Compound 1 or a pharmaceutically acceptable salt thereof once a day is selected to provide a substantial reduction in tremor. In some embodiments, about 90 mg of Compound 1 or a pharmaceutically acceptable salt thereof once a day is selected to provide a substantial reduction in tremor. In some embodiments, about 95 mg of Compound 1 or a pharmaceutically acceptable salt thereof once a day is selected to provide a substantial reduction in tremor. In some embodiments, about 100 mg of Compound 1 or a pharmaceutically acceptable salt thereof once a day is selected to provide a substantial reduction in tremor. In some embodiments, about 105 mg of Compound 1 or a pharmaceutically acceptable salt thereof once a day is selected to provide a substantial reduction in tremor. In some embodiments, about 110 mg of Compound 1 or a pharmaceutically acceptable salt thereof once a day is selected to provide a substantial reduction in tremor. In some embodiments, about 115 mg of Compound 1 or a pharmaceutically acceptable salt thereof once a day is selected to provide a substantial reduction in tremor. In some embodiments, about 120 mg of Compound 1 or a pharmaceutically acceptable salt thereof once a day is selected to provide a substantial reduction in tremor.

In some embodiments, the total daily dose of Compound 1 or a pharmaceutically acceptable salt thereof is at least about 5 mg a day for the treatment of essential tremor. In some embodiments, the total daily dose of Compound 1 or a pharmaceutically acceptable salt thereof is at least about 10 mg a day for the treatment of essential tremor. In some embodiments, the total daily dose of Compound 1 or a pharmaceutically acceptable salt thereof is at least about 15 mg a day for the treatment of essential tremor. In some embodiments, the total daily dose of Compound 1 or a pharmaceutically acceptable salt thereof is at least about 20 mg a day for the treatment of essential tremor. In some embodiments, the total daily dose of Compound 1 or a pharmaceutically acceptable salt thereof is at least about 25 mg a day for the treatment of essential tremor. In some embodiments, the total daily dose of Compound 1 or a pharmaceutically acceptable salt thereof is at least about 30 mg a day for the treatment of essential tremor. In some embodiments, the total daily dose of Compound 1 or a pharmaceutically acceptable salt thereof is at least about 35 mg a day for the treatment of essential tremor. In some embodiments, the total daily dose of Compound 1 or a pharmaceutically acceptable salt thereof is at least about 40 mg a day for the treatment of essential tremor. In some embodiments, the total daily dose of Compound 1 or a pharmaceutically acceptable salt thereof is at least about 45 mg a day for the treatment of essential tremor. In some embodiments, the total daily dose of Compound 1 or a pharmaceutically acceptable salt thereof is at least about 50 mg a day for the treatment of essential tremor. In some embodiments, the total daily dose of Compound 1 or a pharmaceutically acceptable salt thereof is at least about 55 mg a day for the treatment of essential tremor. In some embodiments, the total daily dose of Compound 1 or a pharmaceutically acceptable salt thereof is at least about 60 mg a day for the treatment of essential tremor. In some embodiments, the total daily dose of Compound 1 or a pharmaceutically acceptable salt thereof is at least about 65 mg a day for the treatment of essential tremor. In some embodiments, the total daily dose of Compound 1 or a pharmaceutically acceptable salt thereof is at least about 70 mg a day for the treatment of essential tremor. In some embodiments, the total daily dose of Compound 1 or a pharmaceutically acceptable salt thereof is at least about 75 mg a day for the treatment of essential tremor. In some embodiments, the total daily dose of Compound 1 or a pharmaceutically acceptable salt thereof is at least about 80 mg a day for the treatment of essential tremor. In some embodiments, the total daily dose of Compound 1 or a pharmaceutically acceptable salt thereof is at least about 85 mg a day for the treatment of essential tremor. In some embodiments, the total daily dose of Compound 1 or a pharmaceutically acceptable salt thereof is at least about 90 mg a day for the treatment of essential tremor. In some embodiments, the total daily dose of Compound 1 or a pharmaceutically acceptable salt thereof is at least about 95 mg a day for the treatment of essential tremor. In some embodiments, the total daily dose of Compound 1 or a pharmaceutically acceptable salt thereof is at least about 100 mg a day for the treatment of essential tremor. In some embodiments, the total daily dose of Compound 1 or a pharmaceutically acceptable salt thereof is at least about 105 mg a day for the treatment of essential tremor. In some embodiments, the total daily dose of Compound 1 or a pharmaceutically acceptable salt thereof is at least about 110 mg a day for the treatment of essential tremor. In some embodiments, the total daily dose of Compound 1 or a pharmaceutically acceptable salt thereof is at least about 115 mg a day for the treatment of essential tremor. In some embodiments, the total daily dose of Compound 1 or a pharmaceutically acceptable salt thereof is at least about 120 mg a day for the treatment of essential tremor.

In some embodiments, the total daily dose of Compound 1 or a pharmaceutically acceptable salt thereof is about 5 mg a day for the treatment of essential tremor. In some embodiments, the total daily dose of Compound 1 or a pharmaceutically acceptable salt thereof is about 10 mg a day for the treatment of essential tremor. In some embodiments, the total daily dose of Compound 1 or a pharmaceutically acceptable salt thereof is about 15 mg a day for the treatment of essential tremor. In some embodiments, the total daily dose of Compound 1 or a pharmaceutically acceptable salt thereof is about 20 mg a day for the treatment of essential tremor. In some embodiments, the total daily dose of Compound 1 or a pharmaceutically acceptable salt thereof is about 25 mg a day for the treatment of essential tremor. In some embodiments, the total daily dose of Compound 1 or a pharmaceutically acceptable salt thereof is about 30 mg a day for the treatment of essential tremor. In some embodiments, the total daily dose of Compound 1 or a pharmaceutically acceptable salt thereof is about 35 mg a day for the treatment of essential tremor. In some embodiments, the total daily dose of Compound 1 or a pharmaceutically acceptable salt thereof is about 40 mg a day for the treatment of essential tremor. In some embodiments, the total daily dose of Compound 1 or a pharmaceutically acceptable salt thereof is about 45 mg a day for the treatment of essential tremor. In some embodiments, the total daily dose of Compound 1 or a pharmaceutically acceptable salt thereof is about 50 mg a day for the treatment of essential tremor. In some embodiments, the total daily dose of Compound 1 or a pharmaceutically acceptable salt thereof is about 55 mg a day for the treatment of essential tremor. In some embodiments, the total daily dose of Compound 1 or a pharmaceutically acceptable salt thereof is about 60 mg a day for the treatment of essential tremor. In some embodiments, the total daily dose of Compound 1 or a pharmaceutically acceptable salt thereof is about 65 mg a day for the treatment of essential tremor. In some embodiments, the total daily dose of Compound 1 or a pharmaceutically acceptable salt thereof is about 70 mg a day for the treatment of essential tremor. In some embodiments, the total daily dose of Compound 1 or a pharmaceutically acceptable salt thereof is about 75 mg a day for the treatment of essential tremor. In some embodiments, the total daily dose of Compound 1 or a pharmaceutically acceptable salt thereof is about 80 mg a day for the treatment of essential tremor. In some embodiments, the total daily dose of Compound 1 or a pharmaceutically acceptable salt thereof is about 85 mg a day for the treatment of essential tremor. In some embodiments, the total daily dose of Compound 1 or a pharmaceutically acceptable salt thereof is about 90 mg a day for the treatment of essential tremor. In some embodiments, the total daily dose of Compound 1 or a pharmaceutically acceptable salt thereof is about 95 mg a day for the treatment of essential tremor. In some embodiments, the total daily dose of Compound 1 or a pharmaceutically acceptable salt thereof is about 100 mg a day for the treatment of essential tremor. In some embodiments, the total daily dose of Compound 1 or a pharmaceutically acceptable salt thereof is about 105 mg a day for the treatment of essential tremor. In some embodiments, the total daily dose of Compound 1 or a pharmaceutically acceptable salt thereof is about 110 mg a day for the treatment of essential tremor. In some embodiments, the total daily dose of Compound 1 or a pharmaceutically acceptable salt thereof is about 115 mg a day for the treatment of essential tremor. In some embodiments, the total daily dose of Compound 1 or a pharmaceutically acceptable salt thereof is about 120 mg a day for the treatment of essential tremor.

In some embodiments, about 5 mg of Compound 1 or a pharmaceutically acceptable salt thereof once a day is selected to provide a substantial reduction in essential tremor. In some embodiments, about 5 mg of Compound 1 or a pharmaceutically acceptable salt thereof twice a day is selected to provide a substantial reduction in essential tremor. In some embodiments, about 10 mg of Compound 1 or a pharmaceutically acceptable salt thereof once a day is selected to provide a substantial reduction in essential tremor. In some embodiments, about 10 mg of Compound 1 or a pharmaceutically acceptable salt thereof twice a day is selected to provide a substantial reduction in essential tremor. In some embodiments, about 15 mg of Compound 1 or a pharmaceutically acceptable salt thereof once a day is selected to provide a substantial reduction in essential tremor. In some embodiments, about 15 mg of Compound 1 or a pharmaceutically acceptable salt thereof twice a day is selected to provide a substantial reduction in essential tremor. In some embodiments, about 20 mg of Compound 1 or a pharmaceutically acceptable salt thereof once a day is selected to provide a substantial reduction in essential tremor. In some embodiments, about 20 mg of Compound 1 or a pharmaceutically acceptable salt thereof twice a day is selected to provide a substantial reduction in essential tremor. In some embodiments, about 25 mg of Compound 1 or a pharmaceutically acceptable salt thereof once a day is selected to provide a substantial reduction in essential tremor. In some embodiments, about 25 mg of Compound 1 or a pharmaceutically acceptable salt thereof twice a day is selected to provide a substantial reduction in essential tremor. In some embodiments, about 30 mg of Compound 1 or a pharmaceutically acceptable salt thereof once a day is selected to provide a substantial reduction in essential tremor. In some embodiments, about 30 mg of Compound 1 or a pharmaceutically acceptable salt thereof twice a day is selected to provide a substantial reduction in essential tremor. In some embodiments, about 35 mg of Compound 1 or a pharmaceutically acceptable salt thereof once a day is selected to provide a substantial reduction in essential tremor. In some embodiments, about 35 mg of Compound 1 or a pharmaceutically acceptable salt thereof twice a day is selected to provide a substantial reduction in essential tremor. In some embodiments, about 40 mg of Compound 1 or a pharmaceutically acceptable salt thereof once a day is selected to provide a substantial reduction in essential tremor. In some embodiments, about 40 mg of Compound 1 or a pharmaceutically acceptable salt thereof twice a day is selected to provide a substantial reduction in essential tremor. In some embodiments, about 45 mg of Compound 1 or a pharmaceutically acceptable salt thereof once a day is selected to provide a substantial reduction in essential tremor. In some embodiments, about 45 mg of Compound 1 or a pharmaceutically acceptable salt thereof twice a day is selected to provide a substantial reduction in essential tremor. In some embodiments, about 50 mg of Compound 1 or a pharmaceutically acceptable salt thereof once a day is selected to provide a substantial reduction in essential tremor. In some embodiments, about 50 mg of Compound 1 or a pharmaceutically acceptable salt thereof twice a day is selected to provide a substantial reduction in essential tremor. In some embodiments, about 55 mg of Compound 1 or a pharmaceutically acceptable salt thereof once a day is selected to provide a substantial reduction in essential tremor. In some embodiments, about 55 mg of Compound 1 or a pharmaceutically acceptable salt thereof twice a day is selected to provide a substantial reduction in essential tremor. In some embodiments, about 60 mg of Compound 1 or a pharmaceutically acceptable salt thereof once a day is selected to provide a substantial reduction in essential tremor. In some embodiments, about 60 mg of Compound 1 or a pharmaceutically acceptable salt thereof twice a day is selected to provide a substantial reduction in essential tremor. In some embodiments, about 65 mg of Compound 1 or a pharmaceutically acceptable salt thereof once a day is selected to provide a substantial reduction in essential tremor. In some embodiments, about 70 mg of Compound 1 or a pharmaceutically acceptable salt thereof once a day is selected to provide a substantial reduction in essential tremor. In some embodiments, about 75 mg of Compound 1 or a pharmaceutically acceptable salt thereof once a day is selected to provide a substantial reduction in essential tremor. In some embodiments, about 80 mg of Compound 1 or a pharmaceutically acceptable salt thereof once a day is selected to provide a substantial reduction in essential tremor. In some embodiments, about 85 mg of Compound 1 or a pharmaceutically acceptable salt thereof once a day is selected to provide a substantial reduction in essential tremor. In some embodiments, about 90 mg of Compound 1 or a pharmaceutically acceptable salt thereof once a day is selected to provide a substantial reduction in essential tremor. In some embodiments, about 95 mg of Compound 1 or a pharmaceutically acceptable salt thereof once a day is selected to provide a substantial reduction in essential tremor. In some embodiments, about 100 mg of Compound 1 or a pharmaceutically acceptable salt thereof once a day is selected to provide a substantial reduction in essential tremor. In some embodiments, about 105 mg of Compound 1 or a pharmaceutically acceptable salt thereof once a day is selected to provide a substantial reduction in essential tremor. In some embodiments, about 110 mg of Compound 1 or a pharmaceutically acceptable salt thereof once a day is selected to provide a substantial reduction in essential tremor. In some embodiments, about 115 mg of Compound 1 or a pharmaceutically acceptable salt thereof once a day is selected to provide a substantial reduction in essential tremor. In some embodiments, about 120 mg of Compound 1 or a pharmaceutically acceptable salt thereof once a day is selected to provide a substantial reduction in essential tremor.

According to some embodiments disclosed herein, the dosing frequency and dose amount per administration of Compound 1 or a pharmaceutically acceptable salt thereof are selected to provide therapeutic effects for the treatment of the various forms of tremor disclosed herein.

In some embodiments, the dosing frequency and dose amount per administration of Compound 1 or a pharmaceutically acceptable salt thereof are selected to provide therapeutic effects for the treatment of tremor. Methods of diagnosing tremor are disclosed in the art, such as set forth in the Essential Tremor Rating Scale (TETRAS), as described by R. J. Elble, J. Neurol. Neuromedicine (2016) 1(4): 34-38 (herein incorporated by reference in its entirety for all purposes).

In some embodiments, the dosing frequency, dose amount per administration of Compound 1 or a pharmaceutically acceptable salt thereof, and dosing time are selected to provide a therapeutic effect for the treatment of depression or anxiety that are comorbid with tremor, particularly when the administration of Compound 1 or a pharmaceutically acceptable salt thereof is in combination with administration of a T-type calcium channel blocker, for example Compound 2 or a pharmaceutically acceptable salt thereof.

In some embodiments, the total daily dose of Compound 1 or a pharmaceutically acceptable salt thereof is at least about 5 mg a day for the treatment of fibromyalgia and related musculoskeletal conditions. In some embodiments, the total daily dose of Compound 1 or a pharmaceutically acceptable salt thereof is at least about 10 mg a day for the treatment of fibromyalgia and related musculoskeletal conditions. In some embodiments, the total daily dose of Compound 1 or a pharmaceutically acceptable salt thereof is at least about 15 mg a day for the treatment of fibromyalgia and related musculoskeletal conditions. In some embodiments, the total daily dose of Compound 1 or a pharmaceutically acceptable salt thereof is at least about 20 mg a day for the treatment of fibromyalgia and related musculoskeletal conditions. In some embodiments, the total daily dose of Compound 1 or a pharmaceutically acceptable salt thereof is at least about 25 mg a day for the treatment of fibromyalgia and related musculoskeletal conditions. In some embodiments, the total daily dose of Compound 1 or a pharmaceutically acceptable salt thereof is at least about 30 mg a day for the treatment of fibromyalgia and related musculoskeletal conditions. In some embodiments, the total daily dose of Compound 1 or a pharmaceutically acceptable salt thereof is at least about 35 mg a day for the treatment of fibromyalgia and related musculoskeletal conditions. In some embodiments, the total daily dose of Compound 1 or a pharmaceutically acceptable salt thereof is at least about 40 mg a day for the treatment of fibromyalgia and related musculoskeletal conditions. In some embodiments, the total daily dose of Compound 1 or a pharmaceutically acceptable salt thereof is at least about 45 mg a day for the treatment of fibromyalgia and related musculoskeletal conditions. In some embodiments, the total daily dose of Compound 1 or a pharmaceutically acceptable salt thereof is at least about 50 mg a day for the treatment of fibromyalgia and related musculoskeletal conditions. In some embodiments, the total daily dose of Compound 1 or a pharmaceutically acceptable salt thereof is at least about 55 mg a day for the treatment of fibromyalgia and related musculoskeletal conditions. In some embodiments, the total daily dose of Compound 1 or a pharmaceutically acceptable salt thereof is at least about 60 mg a day for the treatment of fibromyalgia and related musculoskeletal conditions. In some embodiments, the total daily dose of Compound 1 or a pharmaceutically acceptable salt thereof is at least about 65 mg a day for the treatment of fibromyalgia and related musculoskeletal conditions. In some embodiments, the total daily dose of Compound 1 or a pharmaceutically acceptable salt thereof is at least about 70 mg a day for the treatment of fibromyalgia and related musculoskeletal conditions. In some embodiments, the total daily dose of Compound 1 or a pharmaceutically acceptable salt thereof is at least about 75 mg a day for the treatment of fibromyalgia and related musculoskeletal conditions. In some embodiments, the total daily dose of Compound 1 or a pharmaceutically acceptable salt thereof is at least about 80 mg a day for the treatment of fibromyalgia and related musculoskeletal conditions. In some embodiments, the total daily dose of Compound 1 or a pharmaceutically acceptable salt thereof is at least about 85 mg a day for the treatment of fibromyalgia and related musculoskeletal conditions. In some embodiments, the total daily dose of Compound 1 or a pharmaceutically acceptable salt thereof is at least about 90 mg a day for the treatment of fibromyalgia and related musculoskeletal conditions. In some embodiments, the total daily dose of Compound 1 or a pharmaceutically acceptable salt thereof is at least about 95 mg a day for the treatment of fibromyalgia and related musculoskeletal conditions. In some embodiments, the total daily dose of Compound 1 or a pharmaceutically acceptable salt thereof is at least about 100 mg a day for the treatment of fibromyalgia and related musculoskeletal conditions. In some embodiments, the total daily dose of Compound 1 or a pharmaceutically acceptable salt thereof is at least about 105 mg a day for the treatment of fibromyalgia and related musculoskeletal conditions. In some embodiments, the total daily dose of Compound 1 or a pharmaceutically acceptable salt thereof is at least about 110 mg a day for the treatment of fibromyalgia and related musculoskeletal conditions. In some embodiments, the total daily dose of Compound 1 or a pharmaceutically acceptable salt thereof is at least about 115 mg a day for the treatment of fibromyalgia and related musculoskeletal conditions. In some embodiments, the total daily dose of Compound 1 or a pharmaceutically acceptable salt thereof is at least about 120 mg a day for the treatment of fibromyalgia and related musculoskeletal conditions.

In some embodiments, the total daily dose of Compound 1 or a pharmaceutically acceptable salt thereof is about 5 mg a day for the treatment of fibromyalgia and related musculoskeletal conditions. In some embodiments, the total daily dose of Compound 1 or a pharmaceutically acceptable salt thereof is about 10 mg a day for the treatment of fibromyalgia and related musculoskeletal conditions. In some embodiments, the total daily dose of Compound 1 or a pharmaceutically acceptable salt thereof is about 15 mg a day for the treatment of fibromyalgia and related musculoskeletal conditions. In some embodiments, the total daily dose of Compound 1 or a pharmaceutically acceptable salt thereof is about 20 mg a day for the treatment of fibromyalgia and related musculoskeletal conditions. In some embodiments, the total daily dose of Compound 1 or a pharmaceutically acceptable salt thereof is about 25 mg a day for the treatment of fibromyalgia and related musculoskeletal conditions. In some embodiments, the total daily dose of Compound 1 or a pharmaceutically acceptable salt thereof is about 30 mg a day for the treatment of fibromyalgia and related musculoskeletal conditions. In some embodiments, the total daily dose of Compound 1 or a pharmaceutically acceptable salt thereof is about 35 mg a day for the treatment of fibromyalgia and related musculoskeletal conditions. In some embodiments, the total daily dose of Compound 1 or a pharmaceutically acceptable salt thereof is about 40 mg a day for the treatment of fibromyalgia and related musculoskeletal conditions. In some embodiments, the total daily dose of Compound 1 or a pharmaceutically acceptable salt thereof is about 45 mg a day for the treatment of fibromyalgia and related musculoskeletal conditions. In some embodiments, the total daily dose of Compound 1 or a pharmaceutically acceptable salt thereof is about 50 mg a day for the treatment of fibromyalgia and related musculoskeletal conditions. In some embodiments, the total daily dose of Compound 1 or a pharmaceutically acceptable salt thereof is about 55 mg a day for the treatment of fibromyalgia and related musculoskeletal conditions. In some embodiments, the total daily dose of Compound 1 or a pharmaceutically acceptable salt thereof is about 60 mg a day for the treatment of fibromyalgia and related musculoskeletal conditions. In some embodiments, the total daily dose of Compound 1 or a pharmaceutically acceptable salt thereof is about 65 mg a day for the treatment of fibromyalgia and related musculoskeletal conditions. In some embodiments, the total daily dose of Compound 1 or a pharmaceutically acceptable salt thereof is about 70 mg a day for the treatment of fibromyalgia and related musculoskeletal conditions. In some embodiments, the total daily dose of Compound 1 or a pharmaceutically acceptable salt thereof is about 75 mg a day for the treatment of fibromyalgia and related musculoskeletal conditions. In some embodiments, the total daily dose of Compound 1 or a pharmaceutically acceptable salt thereof is about 80 mg a day for the treatment of fibromyalgia and related musculoskeletal conditions. In some embodiments, the total daily dose of Compound 1 or a pharmaceutically acceptable salt thereof is about 85 mg a day for the treatment of fibromyalgia and related musculoskeletal conditions. In some embodiments, the total daily dose of Compound 1 or a pharmaceutically acceptable salt thereof is about 90 mg a day for the treatment of fibromyalgia and related musculoskeletal conditions. In some embodiments, the total daily dose of Compound 1 or a pharmaceutically acceptable salt thereof is about 95 mg a day for the treatment of fibromyalgia and related musculoskeletal conditions. In some embodiments, the total daily dose of Compound 1 or a pharmaceutically acceptable salt thereof is about 100 mg a day for the treatment of fibromyalgia and related musculoskeletal conditions. In some embodiments, the total daily dose of Compound 1 or a pharmaceutically acceptable salt thereof is about 105 mg a day for the treatment of fibromyalgia and related musculoskeletal conditions. In some embodiments, the total daily dose of Compound 1 or a pharmaceutically acceptable salt thereof is about 110 mg a day for the treatment of fibromyalgia and related musculoskeletal conditions. In some embodiments, the total daily dose of Compound 1 or a pharmaceutically acceptable salt thereof is about 115 mg a day for the treatment of fibromyalgia and related musculoskeletal conditions. In some embodiments, the total daily dose of Compound 1 or a pharmaceutically acceptable salt thereof is about 120 mg a day for the treatment of fibromyalgia and related musculoskeletal conditions.

In some embodiments, about 5 mg of Compound 1 or a pharmaceutically acceptable salt thereof once a day is selected to provide a substantial reduction in fibromyalgia and related musculoskeletal conditions. In some embodiments, about 5 mg of Compound 1 or a pharmaceutically acceptable salt thereof twice a day is selected to provide a substantial reduction in fibromyalgia and related musculoskeletal conditions. In some embodiments, about 10 mg of Compound 1 or a pharmaceutically acceptable salt thereof once a day is selected to provide a substantial reduction in fibromyalgia and related musculoskeletal conditions. In some embodiments, about 10 mg of Compound 1 or a pharmaceutically acceptable salt thereof twice a day is selected to provide a substantial reduction in fibromyalgia and related musculoskeletal conditions. In some embodiments, about 15 mg of Compound 1 or a pharmaceutically acceptable salt thereof once a day is selected to provide a substantial reduction in fibromyalgia and related musculoskeletal conditions. In some embodiments, about 15 mg of Compound 1 or a pharmaceutically acceptable salt thereof twice a day is selected to provide a substantial reduction in fibromyalgia and related musculoskeletal conditions. In some embodiments, about 20 mg of Compound 1 or a pharmaceutically acceptable salt thereof once a day is selected to provide a substantial reduction in fibromyalgia and related musculoskeletal conditions. In some embodiments, about 20 mg of Compound 1 or a pharmaceutically acceptable salt thereof twice a day is selected to provide a substantial reduction in fibromyalgia and related musculoskeletal conditions. In some embodiments, about 25 mg of Compound 1 or a pharmaceutically acceptable salt thereof once a day is selected to provide a substantial reduction in fibromyalgia and related musculoskeletal conditions. In some embodiments, about 25 mg of Compound 1 or a pharmaceutically acceptable salt thereof twice a day is selected to provide a substantial reduction in fibromyalgia and related musculoskeletal conditions. In some embodiments, about 30 mg of Compound 1 or a pharmaceutically acceptable salt thereof once a day is selected to provide a substantial reduction in fibromyalgia and related musculoskeletal conditions. In some embodiments, about 30 mg of Compound 1 or a pharmaceutically acceptable salt thereof twice a day is selected to provide a substantial reduction in fibromyalgia and related musculoskeletal conditions. In some embodiments, about 35 mg of Compound 1 or a pharmaceutically acceptable salt thereof once a day is selected to provide a substantial reduction in fibromyalgia and related musculoskeletal conditions. In some embodiments, about 35 mg of Compound 1 or a pharmaceutically acceptable salt thereof twice a day is selected to provide a substantial reduction in fibromyalgia and related musculoskeletal conditions. In some embodiments, about 40 mg of Compound 1 or a pharmaceutically acceptable salt thereof once a day is selected to provide a substantial reduction in fibromyalgia and related musculoskeletal conditions. In some embodiments, about 40 mg of Compound 1 or a pharmaceutically acceptable salt thereof twice a day is selected to provide a substantial reduction in fibromyalgia and related musculoskeletal conditions. In some embodiments, about 45 mg of Compound 1 or a pharmaceutically acceptable salt thereof once a day is selected to provide a substantial reduction in fibromyalgia and related musculoskeletal conditions. In some embodiments, about 45 mg of Compound 1 or a pharmaceutically acceptable salt thereof twice a day is selected to provide a substantial reduction in fibromyalgia and related musculoskeletal conditions. In some embodiments, about 50 mg of Compound 1 or a pharmaceutically acceptable salt thereof once a day is selected to provide a substantial reduction in fibromyalgia and related musculoskeletal conditions. In some embodiments, about 50 mg of Compound 1 or a pharmaceutically acceptable salt thereof twice a day is selected to provide a substantial reduction in fibromyalgia and related musculoskeletal conditions. In some embodiments, about 55 mg of Compound 1 or a pharmaceutically acceptable salt thereof once a day is selected to provide a substantial reduction in fibromyalgia and related musculoskeletal conditions. In some embodiments, about 55 mg of Compound 1 or a pharmaceutically acceptable salt thereof twice a day is selected to provide a substantial reduction in fibromyalgia and related musculoskeletal conditions. In some embodiments, about 60 mg of Compound 1 or a pharmaceutically acceptable salt thereof once a day is selected to provide a substantial reduction in fibromyalgia and related musculoskeletal conditions. In some embodiments, about 60 mg of Compound 1 or a pharmaceutically acceptable salt thereof twice a day is selected to provide a substantial reduction in fibromyalgia and related musculoskeletal conditions. In some embodiments, about 65 mg of Compound 1 or a pharmaceutically acceptable salt thereof once a day is selected to provide a substantial reduction in fibromyalgia and related musculoskeletal conditions. In some embodiments, about 70 mg of Compound 1 or a pharmaceutically acceptable salt thereof once a day is selected to provide a substantial reduction in fibromyalgia and related musculoskeletal conditions. In some embodiments, about 75 mg of Compound 1 or a pharmaceutically acceptable salt thereof once a day is selected to provide a substantial reduction in fibromyalgia and related musculoskeletal conditions. In some embodiments, about 80 mg of Compound 1 or a pharmaceutically acceptable salt thereof once a day is selected to provide a substantial reduction in fibromyalgia and related musculoskeletal conditions. In some embodiments, about 85 mg of Compound 1 or a pharmaceutically acceptable salt thereof once a day is selected to provide a substantial reduction in fibromyalgia and related musculoskeletal conditions. In some embodiments, about 90 mg of Compound 1 or a pharmaceutically acceptable salt thereof once a day is selected to provide a substantial reduction in fibromyalgia and related musculoskeletal conditions. In some embodiments, about 95 mg of Compound 1 or a pharmaceutically acceptable salt thereof once a day is selected to provide a substantial reduction in fibromyalgia and related musculoskeletal conditions. In some embodiments, about 100 mg of Compound 1 or a pharmaceutically acceptable salt thereof once a day is selected to provide a substantial reduction in fibromyalgia and related musculoskeletal conditions. In some embodiments, about 105 mg of Compound 1 or a pharmaceutically acceptable salt thereof once a day is selected to provide a substantial reduction in fibromyalgia and related musculoskeletal conditions. In some embodiments, about 110 mg of Compound 1 or a pharmaceutically acceptable salt thereof once a day is selected to provide a substantial reduction in fibromyalgia and related musculoskeletal conditions. In some embodiments, about 115 mg of Compound 1 or a pharmaceutically acceptable salt thereof once a day is selected to provide a substantial reduction in fibromyalgia and related musculoskeletal conditions. In some embodiments, about 120 mg of Compound 1 or a pharmaceutically acceptable salt thereof once a day is selected to provide a substantial reduction in fibromyalgia and related musculoskeletal conditions.

According to some embodiments, the substantial reduction in depression provided by the methods of the present disclosure requires treatment for a specified time interval (e.g., at least one week) before the patient experiences substantial reduction of depression (i.e., there is an induction period before the patient experiences a substantial reduction in depression). In some embodiments, after treatment for at least one week, at least two weeks, at least three weeks, at least four weeks, at least five weeks, at least six weeks, at least seven weeks or at least eight weeks, the patient experiences a substantial reduction of depression compared to prior to the treatment. In certain embodiments, after treatment for at least one week the patient experiences a substantial reduction of depression compared to prior to the treatment. According to this embodiment, the substantial reduction in depression may be expressed using any of the methods described herein (for example, decline in total Hamilton Depression Rating Scale (HAM-D) value compared to prior to the treatment, improvement in the Montgomery Åsberg Depression Rating Scale value compared to prior to the treatment, etc.).

According to some embodiments, the substantial reduction in tremor (including essential tremor) provided by the methods of the present disclosure requires treatment for a specified time interval (e.g., at least one week) before the patient experiences substantial reduction of tremor (i.e., there is an induction period before the patient experiences a substantial reduction in tremor). In some embodiments, after treatment for at least one week, at least two weeks, at least three weeks, at least four weeks, at least five weeks, at least six weeks, at least seven weeks or at least eight weeks, the patient experiences a substantial reduction of tremor compared to prior to the treatment. In certain embodiments, after treatment for at least one week the patient experiences a substantial reduction of tremor compared to prior to the treatment. According to this embodiment, the substantial reduction in tremor may be expressed using any of the methods described herein (for example, TETRAS).

According to some embodiments, the substantial reduction in fibromyalgia and related musculoskeletal conditions provided by the methods of the present disclosure requires treatment for a specified time interval (e.g., at least one week) before the patient experiences substantial reduction of tremor (i.e., there is an induction period before the patient experiences a substantial reduction in tremor). In some embodiments, after treatment for at least one week, at least two weeks, at least three weeks, at least four weeks, at least five weeks, at least six weeks, at least seven weeks or at least eight weeks, the patient experiences a substantial reduction of tremor compared to prior to the treatment. In certain embodiments, after treatment for at least one week the patient experiences a substantial reduction of fibromyalgia and related musculoskeletal conditions compared to prior to the treatment. According to this embodiment, the substantial reduction in fibromyalgia and related musculoskeletal conditions may be expressed using any of the methods described herein (for example, the Pain Visual Analog Scale; the VASFIQ (a seven-item scale composed of the Fibromyalgia Impact Questionnaire (FIQ) Visual Analog Scales (VASs) to quantify fibromyalgia global disease severity (Bommershine et al., Ther. Adv. Musculoskelet. Dis. 2011 October; 3(5): 215-226; Numeric Rating Scale for Pain (NRS Pain), McGill Pain Questionnaire (MPQ), Short-Form McGill Pain Questionnaire (SF-MPQ), Chronic Pain Grade Scale (CPGS), Short Form-36 Bodily Pain Scale (SF-36 BPS), and Measure of Intermittent and Constant Osteoarthritis Pain (ICOAP), as described in Hawker et al., Arthritis Care & Research, vol. 63, No. S11, November 2011, pp. S240-S252).

Reduction of depression in patients with depressive conditions can be determined by various methods. In some embodiments, the effectiveness of a dosage regimen can be determined by evaluation via Hamilton Depression Rating Scale (HAM-D). In some embodiments, the effectiveness of a dosage regimen can be determined by evaluation via a Montgomery Åsberg Depression Rating Scale (MADRS). In some embodiments, the effectiveness of a dosage regimen can be determined by evaluation via HAM-D, MADRS, Hamilton Rating Scale for anxiety (HAM-A), Clinical Global Impression (CGI) subscale scores (i.e., Severity of Illness Subscale (CGI-S), Global Improvement Subscale (CGI-I), or Efficacy Index Subscale), Symptoms of Depression Questionnaire (SDQ), Pittsburgh Sleep Quality Index (PSQI), or any combination thereof.

Reduction of comorbid depression in patients with any of the various tremor conditions disclosed herein can also be determined by various methods. In some embodiments, the effectiveness of a dosage regimen can be determined by evaluation via HAM-D. In some embodiments, the effectiveness of a dosage regimen can be determined by evaluation via a MADRS. In some embodiments, the effectiveness of a dosage regimen can be determined by evaluation via HAM-D, MADRS, HAM-A, CGI subscale scores (i.e., CGI-S, CGI-I, or Efficacy Index Subscale), SDQ, PSQI, or any combination thereof.

In some embodiments, the effectiveness of a dosage regimen can be determined by evaluation via a total HAM-D value as a primary efficacy endpoint in association with secondary efficacy endpoints such as the MADRS, HAM-A, CGI-S, CGI-I, SDQ, PSQI, or any combination thereof.

According to some embodiments of the present disclosure, the dosing frequency, dosing schedule and dose amount per administration of Compound 1 or a pharmaceutically acceptable salt thereof are selected to reduce or ameliorate adverse effects associated with the administration of Compound 1 or a pharmaceutically acceptable salt thereof for the treatment of depression selected from major depressive disorder, major depressive disorder with suicidal risk, clinical depression, postnatal or postpartum depression, treatment resistant postpartum depression, perimenopausal depression, PMDD, atypical depression, melancholic depression, PMD, catatonic depression, SAD, persistent depressive disorder (dysthymia), double depression, DPD, RBD, minor depressive disorder, bipolar disorder or manic depressive disorder, bipolar depression with suicidal risk, post-traumatic stress disorders, depression caused by chronic medical conditions, depressive disorder due to another medical condition, treatment-resistant depression, refractory depression, substance/medication induced depressive disorder, depression with anxiety, suicidality, suicidal ideation, or suicidal behavior, adjustment disorder, major depressive disorder in children, major depressive disorder in adolescents, anxiety in children, anxiety in adolescents, or schizophrenia.

According to some embodiments of the present disclosure, the dosing frequency, dosing schedule and dose amount per administration of Compound 1 or pharmaceutically acceptable salts thereof are selected to reduce or ameliorate adverse effects associated with the administration of Compound 1 or a pharmaceutically acceptable salt thereof for the treatment of tremor selected from cerebellar tremor or intention tremor, dystonic tremor, essential tremor, orthostatic tremor, parkinsonian tremor, physiological tremor, psychogenic tremor, or rubral tremor.

According to some embodiments of the present disclosure, the dosing frequency, dosing schedule and dose amount per administration of Compound 1 or pharmaceutically acceptable salts thereof are selected to reduce or ameliorate adverse effects associated with the administration of Compound 1 or a pharmaceutically acceptable salt thereof for the treatment of fibromyalgia or related musculoskeletal conditions.

In some embodiments, the adverse event is somnolence or dizziness. In some embodiments, the adverse event is sedation, lightheadedness, fatigue, euphoria, feeling drunk, feeling abnormal, headache, cognitive disorder, disturbance of attention, memory impairment, slowed cognition, decreased alertness, constipation, or diarrhea.

According to some embodiments of the present disclosure, the dosing frequency and dose amount per administration of Compound 1 or a pharmaceutically acceptable salt thereof are selected to provide therapeutic effects for the treatment of depression selected from major depressive disorder, major depressive disorder with suicidal risk, clinical depression, postnatal or postpartum depression, treatment resistant postpartum depression, perimenopausal depression, PMDD, atypical depression, melancholic depression, PMD, catatonic depression, SAD, persistent depressive disorder (dysthymia), double depression, DPD, RBD, minor depressive disorder, bipolar disorder or manic depressive disorder, bipolar depression with suicidal risk, post-traumatic stress disorders, depression caused by chronic medical conditions, depressive disorder due to another medical condition, treatment-resistant depression, refractory depression, substance/medication induced depressive disorder, depression with anxiety, suicidality, suicidal ideation, suicidal behavior, adjustment disorder, major depressive disorder in children, major depressive disorder in adolescents, anxiety in children, anxiety in adolescents, schizophrenia.

According to some embodiments of the present disclosure, the dosing frequency and dose amount per administration of Compound 1 or a pharmaceutically acceptable salts thereof and optionally Compound 2 or a pharmaceutically acceptable salts thereof are selected to provide therapeutic effects for the treatment of tremor and/or comorbid depression and/or comorbid anxiety. In some embodiments of the present disclosure, the dosing frequency and dose amount per administration of Compound 1 or a pharmaceutically acceptable salts thereof is selected to provide therapeutic effects for the treatment of tremor. In other embodiments of the present disclosure, the dosing frequency and dose amount per administration of Compound 1 or a pharmaceutically acceptable salt thereof and Compound 2 or a pharmaceutically acceptable salt thereof are selected to provide therapeutic effects for the treatment of tremor and comorbid depression and/or comorbid anxiety.

According to some embodiments of the present disclosure, the dosing frequency and dose amount per administration of Compound 1 or a pharmaceutically acceptable salt thereof are selected to provide therapeutic effects for the treatment of major depressive disorder. In certain embodiments, the dosing frequency and amount per administration of Compound 1 or a pharmaceutically acceptable salt thereof are selected to provide for the treatment of moderate major depressive disorder. In certain embodiments, the dosing frequency and amount per administration of Compound 1 or a pharmaceutically acceptable salt thereof are selected to provide for the treatment of severe major depressive disorder. In certain embodiments, the dosing frequency and amount per administration of Compound 1 or a pharmaceutically acceptable salt thereof are selected to provide for the treatment of severe major depressive disorder in a patient having a total HAM-D value of at least 22.

According to some embodiments of the present disclosure, the dosing frequency and dose amount per administration of Compound 1 or a pharmaceutically acceptable salt thereof are selected to provide therapeutic effects for the treatment of child and adolescent depression. In some embodiments, the dosing frequency and dose amount per administration of Compound 1 or a pharmaceutically acceptable salt thereof are selected to provide therapeutic effects for the treatment of child and adolescent depression during puberty. In some embodiments, the dosing frequency and dose amount per administration of Compound 1 or a pharmaceutically acceptable salt thereof are selected to provide therapeutic effects for the treatment of child and adolescent depression during menarche or menarche transition. In some embodiments, the dosing frequency and dose amount per administration of Compound 1 or a pharmaceutically acceptable salt thereof are selected to provide therapeutic effects for the treatment of child and adolescent depression during spermarche or spermarche transition.

According to some embodiments of the present disclosure, the dosing frequency and dose amount per administration of Compound 1 or a pharmaceutically acceptable salt thereof are selected to provide therapeutic effects for the treatment of depression that is refractory to other treatments (i.e., treatment resistant depression).

According to some embodiments of the present disclosure, the dosing frequency and dose amount per administration of Compound 1 or a pharmaceutically acceptable salts thereof are selected to provide therapeutic effects for the treatment of depression that is partially responsive to other antidepressant therapies. According to some embodiments of the present disclosure, the dosing frequency and dose amount per administration of Compound 1 or pharmaceutically acceptable salt thereof are selected to provide an adjunctive treatment for major depression. According to some embodiments of the present disclosure, the dosing frequency and dose amount per administration of Compound 1 or a pharmaceutically acceptable salt thereof are selected to provide therapeutic effects for the treatment of depression that is partially responsive to treatment with SSRIs.

In certain embodiments, the dosing frequency and dose amount per administration of Compound 1 or a pharmaceutically acceptable salt thereof are selected to provide therapeutic effects for the treatment of MDD that is partially responsive to other antidepressant therapies. In certain embodiments, the MDD patient that is partially responsive to other antidepressant therapies is an adult patient meeting DSM-IV criteria for MDD who had had an inadequate response to prior antidepressant therapy (1 to 3 courses) in the current episode and who had demonstrated an inadequate response to 8 weeks of prospective antidepressant therapy. Inadequate response for prospective treatment is defined as less than 50% improvement on the 17-item version of the HAM-D, minimal HAM-D score of 14, and a Clinical Global Impressions Improvement rating of no better than minimal improvement. Inadequate response to prior treatment is defined as less than 50% improvement as perceived by the patient after a minimum of 6 weeks of antidepressant therapy at or above the minimal effective dose. In certain embodiments, the dosing frequency and dose amount per administration of Compound 1 or a pharmaceutically acceptable salt thereof are selected to provide therapeutic effects for the treatment of moderate MDD that is partially responsive to other antidepressant therapies. In certain embodiments, the dosing frequency and dose amount per administration of Compound 1 or a pharmaceutically acceptable salt thereof are selected to provide therapeutic effects for the treatment of severe MDD that is partially responsive to other antidepressant therapies. In certain embodiments, the dosing frequency and dose amount per administration of Compound 1 or a pharmaceutically acceptable salt thereof are selected to provide therapeutic effects for the treatment of MDD that is partially responsive to treatment with SSRIs. In certain embodiments, the dosing frequency and dose amount per administration of Compound 1 or a pharmaceutically acceptable salt thereof are selected to provide therapeutic effects for the treatment of moderate MDD that is partially responsive to treatment with SSRIs. In certain embodiments, the dosing frequency and dose amount per administration of Compound 1 or a pharmaceutically acceptable salt thereof are selected to provide therapeutic effects for the treatment of severe MDD that is partially responsive to treatment with SSRIs.

In certain embodiments, the dosing frequency and dose amount per administration of Compound 1 or a pharmaceutically acceptable salt thereof are selected to provide therapeutic effects for the treatment of MDD in a patient with insomnia. In certain embodiments, the dosing frequency and dose amount per administration of Compound 1 or a pharmaceutically acceptable salt thereof are selected to provide therapeutic effects for the treatment of MDD in a patient with insomnia characterized by difficulties with sleep initiation. In certain embodiments, the dosing frequency and dose amount per administration of Compound 1 or a pharmaceutically acceptable salt thereof are selected to provide therapeutic effects for the treatment of MDD and the treatment of insomnia in an MDD patient with insomnia. In certain embodiments, the dosing frequency and dose amount per administration of Compound 1 or a pharmaceutically acceptable salt thereof are selected to provide therapeutic effects for the treatment of MDD and the treatment of insomnia in an anxious MDD patient with insomnia.

In certain embodiments, after treatment the patient experiences a substantial reduction of MDD and insomnia compared to prior to the treatment. In certain embodiments, after treatment for at least one week the patient experiences a substantial reduction of MDD and insomnia compared to prior to the treatment. According to this embodiment, the substantial reduction in insomnia may be expressed using any of the methods described herein (for example, an improvement in polysomnography parameters, such as a decline in latency to persistent sleep (LPS) compared to prior to the treatment, decline in wake time after sleep onset (WASO) compared to prior to the treatment, etc.), and the substantial reduction in MDD may be expressed using any of the methods described herein (for example, decline in total HAM-D value compared to prior to the treatment, improvement in the MADRS value compared to prior to the treatment, etc.).

The sleep parameters described herein (including wake time after sleep onset, total sleep time, sleep efficiency and latency to persistent sleep) may be measured by polysomnography using methods that are known to those skilled in the art.

Wake time after sleep onset is the wakefulness time occurring after defined sleep onset. In some embodiments, after the treatment the patient experiences a substantial reduction of insomnia that is characterized by at least about a 30% decline in wake time after sleep onset (WASO) compared to prior to the treatment. In some embodiments, the reduction of insomnia is characterized by a decline in WASO ranging from about 30% to about 100%, for example, about 30%, about 40%, about 50%, about 60%, about 70%, about 80%, about 90%, and about 100%, compared to prior to the treatment.

Total Sleep Time (TST) is the amount of actual sleep time in a sleep episode, i.e., the total sleep episode less the awake time. In some embodiments, after the treatment the patient experiences a substantial reduction of insomnia that is characterized by at least about a 30% increase in TST compared to prior to the treatment. In some embodiments, the reduction of insomnia is characterized by an increase in TST ranging from about 30% to about 100%, for example, about 30%, about 40%, about 50%, about 60%, about 70%, about 80%, about 90%, and about 100%, compared to prior to the treatment.

Sleep efficiency (SE) is the percentage of total time in bed actually spent in sleep. An increase in sleep efficiency correlates to an improvement in insomnia. In some embodiments, after the treatment the patient experiences a substantial reduction of insomnia that is characterized by at least about a 30% increase in SE compared to prior to the treatment. In some embodiments, the reduction of insomnia is characterized by an increase in SE value ranging from about 30% to about 100%, for example, about 30%, about 40%, about 50%, about 60%, about 70%, about 80%, about 90%, and about 100%, compared to prior to the treatment.

LPS is the length of time that it takes to accomplish the transition from full wakefulness to sleep. In some embodiments, after the treatment the patient experiences a substantial reduction of insomnia that is characterized by at least about a 30% decrease in LPS compared to prior to the treatment. In some embodiments, the reduction of insomnia is characterized by a decline in LPS value ranging from about 30% to about 100%, for example, about 30%, about 40%, about 50%, about 60%, about 70%, about 80%, about 90%, and about 100%, compared to prior to the treatment.

The Pittsburgh Sleep Quality Index (PSQI) is a 19-item self-report scale that assesses sleep quality and disturbances for the month preceding the assessment (Buysse D. J., The Pittsburgh Sleep Quality Index: a New Instrument for Psychiatric Practice and Research. Psychiatry Res. 1989 May; 28(2), pages 193-213.). The scale generates seven “component” scores that differentiate “poor” from “good” sleep quality: subjective sleep quality, sleep latency, sleep duration, habitual sleep efficiency, sleep disturbances, use of sleeping medication, and daytime dysfunction. The sum of scores for these seven components yields the Global PSQI score. A Global PSQI score of “5” or greater indicates poor sleep quality. In some embodiments, after the treatment the patient experiences a substantial reduction of insomnia that is characterized by at least a one-point decline in Global PSQI score compared to prior to the treatment. In some embodiments, the reduction of insomnia is characterized by a one-point decline in Global PSQI score compared to prior to the treatment. In some embodiments, the reduction of insomnia is characterized by a two-point decline in Global PSQI score compared to prior to the treatment. In some embodiments, the reduction of insomnia is characterized by a three-point decline in Global PSQI score compared to prior to the treatment.

The Epworth Sleepiness Scale (ESS) is also useful for determining the treatment of insomnia. In scoring the ESS, each item is rated on a 4-point scale from 0=would never doze to 3=high chance of dozing. The item scores are summed to produce a total score (range 0-24). A sum of 10 or more from the 8 individual scores reflects above normal daytime sleepiness and need for further evaluation. In some embodiments, after the treatment the patient experiences a substantial reduction of insomnia that is characterized by at least a one-point increase in ESS value compared to prior to the treatment. In some embodiments, the reduction of insomnia is characterized by a one-point increase in ESS value compared to prior to the treatment. In some embodiments, the reduction of insomnia is characterized by a two-point increase in ESS value compared to prior to the treatment. In some embodiments, the reduction of insomnia is characterized by a three-point increase in ESS value compared to prior to the treatment.

The Insomnia Severity Index (ISI) may be used to determine the treatment of insomnia. For example, the Insomnia Severity Index has seven questions, where answers provide a total score ranging from 0 to 28. A total score of 0 to 7 indicates no significant insomnia; 8 to 14 indicates subthreshold insomnia; 15 to 21 indicates clinical insomnia—moderate severity; and 22-28 indicates clinical insomnia—severe. In some embodiments, after the treatment the patient experiences a substantial reduction of insomnia that is characterized by at least a one-point decrease in ISI value compared to prior to the treatment. In some embodiments, the reduction of insomnia is characterized by a one-point decrease in ISI value compared to prior to the treatment. In some embodiments, the reduction of insomnia is characterized by a two-point decrease in ISI value compared to prior to the treatment. In some embodiments, the reduction of insomnia is characterized by a three-point decrease in ISI value compared to prior to the treatment.

The Leeds Sleep Evaluation Questionnaire (LSEQ) may be used to determine the treatment of insomnia. The LSEQ is a 10-item, subjective, self-report measure designed to assess changes in sleep quality over the course of a drug treatment intervention. The LSEQ has four domains: Ease of Initiating Sleep (three items), Quality of Sleep (two items), Ease of Waking (two items) and Behavior Following Wakefulness (three items). LSEQ uses a visual analogue scale where the respondents place markers on a group of 10-cm lines representing the changes have experience in a variety of symptoms since beginning treatment. Lines extend between extremes such as “more difficult than usual” and “easier than usual” (item 6 related to ease of waking). Responses are measured using a 100-mm scale and are then averaged to provide a score for each domain. The average scores can be used to evaluate the efficacy and sleep-related side effects of drug treatment. In some embodiments, after the treatment the patient experiences a substantial reduction of insomnia that is characterized by at least about a 10% improvement in total LSEQ value compared to prior to the treatment. In some embodiments, the reduction of insomnia is characterized by an increase in total LSEQ value ranging from about 10% to about 100%, for example, about 10% about, 20%, about 30%, about 40%, about 50%, about 60%, about 70%, about 80%, about 90%, and about 100%, compared to prior to the treatment. In some embodiments, after the treatment the patient experiences a substantial reduction of insomnia that is characterized by at least about a 10% improvement in Ease of Initiating Sleep LSEQ domain value compared to prior to the treatment. In some embodiments, the reduction of insomnia is characterized by an increase in Ease of Initiating Sleep LSEQ domain value ranging from about 10% to about 100%, for example, about 10% about, 20%, about 30%, about 40%, about 50%, about 60%, about 70%, about 80%, about 90%, and about 100%, compared to prior to the treatment. In some embodiments, after the treatment the patient experiences a substantial reduction of insomnia that is characterized by at least about a 10% improvement in Quality of Sleep LSEQ domain value compared to prior to the treatment. In some embodiments, the reduction of insomnia is characterized by an increase in Quality of Sleep LSEQ domain value ranging from about 10% to about 100%, for example, about 10% about, 20%, about 30%, about 40%, about 50%, about 60%, about 70%, about 80%, about 90%, and about 100%, compared to prior to the treatment. In some embodiments, after the treatment the patient experiences a substantial reduction of insomnia that is characterized by at least about a 10% improvement in Ease of Waking LSEQ domain value compared to prior to the treatment. In some embodiments, the reduction of insomnia is characterized by an increase in Ease of Waking LSEQ domain value ranging from about 10% to about 100%, for example, about 10% about, 20%, about 30%, about 40%, about 50%, about 60%, about 70%, about 80%, about 90%, and about 100%, compared to prior to the treatment. In some embodiments, after the treatment the patient experiences a substantial reduction of insomnia that is characterized by at least about a 10% improvement in Behavior Following Wakefulness LSEQ domain value compared to prior to the treatment. In some embodiments, the reduction of insomnia is characterized by an increase in Behavior Following Wakefulness LSEQ domain value ranging from about 10% to about 100%, for example, about 10% about, 20%, about 30%, about 40%, about 50%, about 60%, about 70%, about 80%, about 90%, and about 100%, compared to prior to the treatment.

The Athens Insomnia Scale (AIS) may be used to determine the treatment of insomnia. The AIS scale assesses the severity of insomnia using the diagnostic criteria set forth by the International Classification of Diseases (ICD-10). The eight-item questionnaire evaluates sleep onset, night and early-morning waking, sleep time, sleep quality, frequency and duration of complaints, distress cause by the experience and interference with daily functions. Respondents use Likert-type scales to show how severely certain sleep difficulties have affected them in the past month. Scores range from 0 (meaning that the item in question has not been a problem to 3 (indicating more acute sleep difficulties), where answers provide a total score ranging from 0 to 24. In some embodiments, after the treatment the patient experiences a substantial reduction of insomnia that is characterized by at least a one-point decrease in total AIS value compared to prior to the treatment. In some embodiments, the reduction of insomnia is characterized by a one-point decrease in total AIS value compared to prior to the treatment. In some embodiments, the reduction of insomnia is characterized by a two-point decrease in total AIS value compared to prior to the treatment. In some embodiments, the reduction of insomnia is characterized by a three-point decrease in total AIS value compared to prior to the treatment. In some embodiments, the reduction of insomnia is characterized by a four-point decrease in total AIS value compared to prior to the treatment. In some embodiments, the reduction of insomnia is characterized by a five-point decrease in total AIS value compared to prior to the treatment. In some embodiments, the reduction of insomnia is characterized by a six-point decrease in total AIS value compared to prior to the treatment. In some embodiments, the reduction of insomnia is characterized by a seven-point decrease in total AIS value compared to prior to the treatment. In some embodiments, the reduction of insomnia is characterized by an eight-point decrease in total AIS value compared to prior to the treatment. In some embodiments, the reduction of insomnia is characterized by a nine-point decrease in total AIS value compared to prior to the treatment. In some embodiments, the reduction of insomnia is characterized by a ten-point decrease in total AIS value compared to prior to the treatment.

The Sleep Quality Index (SQI) may be used to determine the treatment of insomnia. The SQI is an eight-item questionnaire with three categories weighted 0, 1, or 2 for each item (Urponen H., et al. (1991) Sleep Quality and Health: Description of the Sleep Quality Index. In: Peter J. H., Penzel T., Podszus T., von Wichert P. (eds) Sleep and Health Risk. Springer, Berlin, Heidelberg). The value of SQI varies from 0 to 16 with higher scores indicating more severe sleep disturbance. In some embodiments, after the treatment the patient experiences a substantial reduction of insomnia that is characterized by at least a one-point decrease in total SQI value compared to prior to the treatment. In some embodiments, the reduction of insomnia is characterized by a one-point decrease in total SQI value compared to prior to the treatment. In some embodiments, the reduction of insomnia is characterized by a two-point decrease in total SQI value compared to prior to the treatment. In some embodiments, the reduction of insomnia is characterized by a three-point decrease in total SQI value compared to prior to the treatment. In some embodiments, the reduction of insomnia is characterized by a four-point decrease in total SQI value compared to prior to the treatment. In some embodiments, the reduction of insomnia is characterized by a five-point decrease in total SQI value compared to prior to the treatment. In some embodiments, the reduction of insomnia is characterized by a six-point decrease in total SQI value compared to prior to the treatment. In some embodiments, the reduction of insomnia is characterized by a seven-point decrease in total SQI value compared to prior to the treatment. In some embodiments, the reduction of insomnia is characterized by an eight-point decrease in total SQI value compared to prior to the treatment. In some embodiments, the reduction of insomnia is characterized by a nine-point decrease in total SQI value compared to prior to the treatment. In some embodiments, the reduction of insomnia is characterized by a ten-point decrease in total SQI value compared to prior to the treatment.

In some embodiments, the dosing frequency and dose amount per administration of Compound 1 or a pharmaceutically acceptable salt thereof are selected to provide therapeutic effects for the treatment of depression and that do not substantially sedate the patient (i.e., the MDD is treated without substantially sedating the treated patient). In certain embodiments, the dosing frequency and dose amount per administration of Compound 1 or a pharmaceutically acceptable salt thereof are selected to provide therapeutic effects for the treatment of MDD and that do not substantially sedate the patient. In certain embodiments, the dosing frequency and dose amount per administration of Compound 1 or a pharmaceutically acceptable salt thereof are selected to provide therapeutic effects for the treatment of moderate MDD and that do not substantially sedate the patient. In certain embodiments, the dosing frequency and dose amount per administration of Compound 1 or a pharmaceutically acceptable salt thereof are selected to provide therapeutic effects for the treatment of severe MDD and that do not substantially sedate the patient.

In some embodiments, the dosing frequency and dose amount per administration of Compound 1 or a pharmaceutically acceptable salt thereof are selected to provide therapeutic effects for the treatment of tremor (including essential tremor) and that do not substantially sedate the patient (i.e., the tremor is treated without substantially sedating the treated patient). In certain embodiments, the dosing frequency and dose amount per administration of Compound 1 or a pharmaceutically acceptable salt thereof are selected to provide therapeutic effects for the treatment of tremor and that do not substantially sedate the patient. In certain embodiments, the dosing frequency and dose amount per administration of Compound 1 or a pharmaceutically acceptable salt thereof are selected to provide therapeutic effects for the treatment of essential tremor and that do not substantially sedate the patient. In certain embodiments, the dosing frequency and dose amount per administration of Compound 1 or a pharmaceutically acceptable salt thereof and Compound 2 or a pharmaceutically acceptable salt thereof are selected to provide therapeutic effects for the treatment of tremor (including essential tremor) and comorbid depression and/or anxiety and that do not substantially sedate the patient.

In some embodiments, the dosing frequency and dose amount per administration of Compound 1 or a pharmaceutically acceptable salt thereof are selected to provide therapeutic effects for the treatment of fibromyalgia and related musculoskeletal conditions and that do not substantially sedate the patient (i.e., the fibromyalgia is treated without substantially sedating the treated patient). In certain embodiments, the dosing frequency and dose amount per administration of Compound 1 or a pharmaceutically acceptable salt thereof are selected to provide therapeutic effects for the treatment of fibromyalgia and that do not substantially sedate the patient.

A patient's sedation level may be measured using methods that are known to those skilled in the art. For example, sedation level may be measured using the Modified Observer's Assessment of Alertness/Sedation Scale (G. Schmidt, et al., Comparative Evaluation of the Datex-Ohmeda S/5 Entropy Module and the Bispectral Index® Monitor during Propofol-Remifentanil Anesthesia. Anesthesiology 2004; 101:1283-90) or the Stanford Sleepiness Scale (Quantification of Sleepiness: A New Approach. Psychophysiology Volume 10, Issue 4, pages 431-436, July 1973).

In certain embodiments, the dosing frequency and dose amount per administration of Compound 1 or a pharmaceutically acceptable salt thereof are selected to provide therapeutic effects for the treatment of depression and a Modified Observer's Assessment of Alertness/Sedation Scale (MOAS/S) score of at least 4.0. In certain embodiments, the dosing frequency and dose amount per administration of Compound 1 or a pharmaceutically acceptable salt thereof are selected to provide therapeutic effects for the treatment of depression and a MOAS/S score of 4. In certain embodiments, the dosing frequency and dose amount per administration of Compound 1 or a pharmaceutically acceptable salt thereof are selected to provide therapeutic effects for the treatment of depression and a MOAS/S score of 5.

In certain embodiments, the dosing frequency and dose amount per administration of Compound 1 or a pharmaceutically acceptable salt thereof are selected to provide therapeutic effects for the treatment of depression and a Stanford Sleepiness Scale Score of less than 3.0. In certain embodiments, the dosing frequency and dose amount per administration of Compound 1 or a pharmaceutically acceptable salt thereof are selected to provide therapeutic effects for the treatment of depression and a Stanford Sleepiness Scale Score of 2. In certain embodiments, the dosing frequency and dose amount per administration of Compound 1 or a pharmaceutically acceptable salt thereof are selected to provide therapeutic effects for the treatment of depression and a Stanford Sleepiness Scale Score of 1.

Anxious distress has been noted as a prominent feature of both bipolar and major depressive disorder in both primary care and specialty mental health settings. High levels of anxiety have been associated with higher suicide risk, longer duration of illness, and greater likelihood of treatment non-response. In certain embodiments, the dosing frequency and dose amount per administration of Compound 1 or a pharmaceutically acceptable salt thereof are selected to provide therapeutic effects for the treatment of MDD in a patient with anxious distress. In certain embodiments, the dosing frequency and dose amount per administration of Compound 1 or a pharmaceutically acceptable salt thereof are selected to provide therapeutic effects for the treatment of MDD in a patient with mild anxious distress. In certain embodiments, the dosing frequency and dose amount per administration of Compound 1 or a pharmaceutically acceptable salt thereof are selected to provide therapeutic effects for the treatment of MDD in a patient with moderate anxious distress. In certain embodiments, the dosing frequency and dose amount per administration of Compound 1 or a pharmaceutically acceptable salt thereof are selected to provide therapeutic effects for the treatment of MDD in a patient with moderate-severe anxious distress. In certain embodiments, the dosing frequency and dose amount per administration of Compound 1 or a pharmaceutically acceptable salt thereof are selected to provide therapeutic effects for the treatment of MDD in a patient with severe anxious distress.

In some embodiments, the reduction of anxious distress is characterized by an at least one classification reduction in anxious distress severity classification compared to prior to the treatment (e.g., moderate anxious distress to mild anxious distress). In some embodiments, the reduction of anxious distress is characterized by an at least two classification reduction in anxious distress severity classification compared to prior to the treatment. In some embodiments, the reduction of anxious distress is characterized by an at least three classification reduction in anxious distress severity classification compared to prior to the treatment. In some embodiments, the reduction of anxious distress is characterized by a one-classification reduction in anxious distress severity classification compared to prior to the treatment. In some embodiments, the reduction of anxious distress is characterized by a two-classification reduction in anxious distress severity classification compared to prior to the treatment. In some embodiments, the reduction of anxious distress is characterized by a three-classification reduction in anxious distress severity classification compared to prior to the treatment. In some embodiments, the reduction of anxious distress is characterized by a four-classification reduction in anxious distress severity classification compared to prior to the treatment.

In certain embodiments, the dosing frequency and dose amount per administration of Compound 1 or a pharmaceutically acceptable salt thereof are selected to provide therapeutic effects for the treatment of tremor and a MOAS/S score of at least 4.0. In certain embodiments, the dosing frequency and dose amount per administration of Compound 1 or a pharmaceutically acceptable salt thereof are selected to provide therapeutic effects for the treatment of tremor and a MOAS/S score of 4. In certain embodiments, the dosing frequency and dose amount per administration of Compound 1 or a pharmaceutically acceptable salt thereof are selected to provide therapeutic effects for the treatment of tremor and a MOAS/S score of 5.

In certain embodiments, the dosing frequency and dose amount per administration of Compound 1 or a pharmaceutically acceptable salt thereof are selected to provide therapeutic effects for the treatment of tremor and a Stanford Sleepiness Scale Score of less than 3.0. In certain embodiments, the dosing frequency and dose amount per administration of Compound 1 or a pharmaceutically acceptable salt thereof are selected to provide therapeutic effects for the treatment of tremor and a Stanford Sleepiness Scale Score of 2. In certain embodiments, the dosing frequency and dose amount per administration of Compound 1 or a pharmaceutically acceptable salt thereof are selected to provide therapeutic effects for the treatment of tremor and a Stanford Sleepiness Scale Score of 1.

Depression and anxiety have been noted as a comorbid feature of tremor (e.g., essential tremor). High levels of depression and anxiety have been associated with lower quality of life, increased psychiatric disability, and greater use of healthcare resources. In certain embodiments, the dosing frequency and dose amount per administration of Compound 1 and optionally Compound 2 are selected to provide therapeutic effects for the treatment of tremor in a patient with comorbid depression and/or anxiety.

In some embodiments, the reduction of comorbid depression and/or anxiety is characterized by an at least one classification reduction in depression and/or anxiety classifications compared to prior to the treatment. In some embodiments, the reduction of depression and/or anxiety is characterized by an at least two classification reduction in depression and/or anxiety severity classifications compared to prior to the treatment. In some embodiments, the reduction of depression and/or anxiety is characterized by an at least three classification reduction in depression and/or anxiety severity classification compared to prior to the treatment. In some embodiments, the reduction of depression and/or anxiety is characterized by a one-classification reduction in depression and/or anxiety severity classification compared to prior to the treatment. In some embodiments, the reduction of depression and/or anxiety is characterized by a two-classification reduction in depression and/or anxiety severity classification compared to prior to the treatment. In some embodiments, the reduction of depression and/or anxiety is characterized by a three-classification reduction in depression and/or anxiety severity classification compared to prior to the treatment. In some embodiments, the reduction of depression and/or anxiety is characterized by a four-classification reduction in depression and/or anxiety severity classification compared to prior to the treatment.

The HAM-D is a depression rating scale consisting of 17 items, eight items are scored on a 5-point scale (ranging from 0 to 4), and 9 items are scores on a 3-point scale (ranging from 0 to 2). The total score of the 17 items ranges from 0 to 50 with higher scores indicating greater depression. The total score of the 17 items is used to categorize the severity of depression: normal (total score between 0 and 7), mild depression (total score between 8 and 13), moderate depression (total score between 14-18), severe depression (total score between 19-22). Therefore, a decrease in the total score or on individual item scores indicates improvement Hamilton, M. A Rating Scale for Depression, Journal of Neurology, Neurosurgery, and Psychiatry. (1960) 23, pages 56-62.

In some embodiments, after the treatment the patient experiences a substantial reduction of depression that is characterized by at least about a 30% decline in total HAM-D value compared to prior to the treatment. In some embodiments, the reduction of depression is characterized by a decline in HAM-D value ranging from about 30% to about 100%, for example, about 30%, about 40%, about 50%, about 60%, about 70%, about 80%, about 90%, and about 100%, compared to prior to the treatment.

In some embodiments, after the treatment the patient experiences a substantial reduction of depression that is characterized by at least a one-point decline in HAM-D value compared to prior to the treatment. In some embodiments, the reduction of depression is characterized by a decline in HAM-D value ranging from about one point to about twenty points, for example, about one point, about two points, about three points, about four points, about five points, about six points, about seven points, about eight points, about nine points, about ten points, about eleven points, about twelve points, about thirteen points, about fourteen points, about fifteen points, about sixteen points, about seventeen points, about eighteen points, about nineteen points, and about twenty points compared to prior to the treatment. In some embodiments, the reduction of depression is characterized by a decline HAM-D value of about two points. In some embodiments, the reduction of depression is characterized by a decline in HAM-D value of about three points. In some embodiments, the reduction of depression is characterized by a decline in HAM-D value of about four points. In some embodiments, the reduction of depression is characterized by a decline in HAM-D value of about five points. In some embodiments, the reduction of depression is characterized by a decline HAM-D value of about six points. In some embodiments, the reduction of depression is characterized by a decline in HAM-D value of about seven points. In some embodiments, the reduction of depression is characterized by a decline in HAM-D value of about eight points. In some embodiments, the reduction of depression is characterized by a decline in HAM-D value of about nine points. In some embodiments, the reduction of depression is characterized by a decline HAM-D value of about ten points. In some embodiments, the reduction of depression is characterized by a decline in HAM-D value of about eleven points. In some embodiments, the reduction of depression is characterized by a decline in HAM-D value of about twelve points. In some embodiments, the reduction of depression is characterized by a decline in HAM-D value of about thirteen points. In some embodiments, the reduction of depression is characterized by a decline HAM-D value of about fourteen points. In some embodiments, the reduction of depression is characterized by a decline in HAM-D value of about fifteen points. In some embodiments, the reduction of depression is characterized by a decline in HAM-D value of about sixteen points. In some embodiments, the reduction of depression is characterized by a decline in HAM-D value of about seventeen points. In some embodiments, the reduction of depression is characterized by a decline HAM-D value of about eighteen points. In some embodiments, the reduction of depression is characterized by a decline in HAM-D value of about nineteen points. In some embodiments, the reduction of depression is characterized by a decline in HAM-D value of about twenty points.

In some embodiments, after the treatment the patient experiences a substantial reduction of depression that is characterized by at least a one category change in HAM-D severity classification compared to prior to the treatment. In some embodiments, the reduction of depression is characterized by a one category change HAM-D severity classification compared to prior to the treatment. In some embodiments, the reduction of depression is characterized by a two-category change HAM-D severity classification compared to prior to the treatment. In some embodiments, the reduction of depression is characterized by a three-category change HAM-D severity classification compared to prior to the treatment. In certain embodiments, the reduction of depression is characterized by remission according to HAM-D value after said treatment (i.e., total HAM-D value of 7 or less).

In some embodiments, after the treatment the patient experiences a substantial reduction of comorbid depression that is characterized by at least about a 30% decline in total Hamilton Depression Rating Scale (HAM-D) value compared to prior to the treatment. In some embodiments, the reduction of comorbid depression is characterized by a decline in HAM-D value ranging from about 30% to about 100%, for example, about 30%, about 40%, about 50%, about 60%, about 70%, about 80%, about 90%, and about 100%, compared to prior to the treatment.

In some embodiments, after the treatment the patient experiences a substantial reduction of comorbid depression that is characterized by at least a one-point decline in HAM-D value compared to prior to the treatment. In some embodiments, the reduction of comorbid depression is characterized by a decline in HAM-D value ranging from about one point to about twenty points, for example, about one point, about two points, about three points, about four points, about five points, about six points, about seven points, about eight points, about nine points, about ten points, about eleven points, about twelve points, about thirteen points, about fourteen points, about fifteen points, about sixteen points, about seventeen points, about eighteen points, about nineteen points, and about twenty points compared to prior to the treatment. In some embodiments, the reduction of comorbid depression is characterized by a decline HAM-D value of about two points. In some embodiments, the reduction of comorbid depression is characterized by a decline in HAM-D value of about three points. In some embodiments, the reduction of comorbid depression is characterized by a decline in HAM-D value of about four points. In some embodiments, the reduction of comorbid depression is characterized by a decline in HAM-D value of about five points. In some embodiments, the reduction of comorbid depression is characterized by a decline HAM-D value of about six points. In some embodiments, the reduction of comorbid depression is characterized by a decline in HAM-D value of about seven points. In some embodiments, the reduction of comorbid depression is characterized by a decline in HAM-D value of about eight points. In some embodiments, the reduction of comorbid depression is characterized by a decline in HAM-D value of about nine points. In some embodiments, the reduction of comorbid depression is characterized by a decline HAM-D value of about ten points. In some embodiments, the reduction of comorbid depression is characterized by a decline in HAM-D value of about eleven points. In some embodiments, the reduction of comorbid depression is characterized by a decline in HAM-D value of about twelve points. In some embodiments, the reduction of comorbid depression is characterized by a decline in HAM-D value of about thirteen points. In some embodiments, the reduction of comorbid depression is characterized by a decline HAM-D value of about fourteen points. In some embodiments, the reduction of comorbid depression is characterized by a decline in HAM-D value of about fifteen points. In some embodiments, the reduction of comorbid depression is characterized by a decline in HAM-D value of about sixteen points. In some embodiments, the reduction of comorbid depression is characterized by a decline in HAM-D value of about seventeen points. In some embodiments, the reduction of comorbid depression is characterized by a decline HAM-D value of about eighteen points. In some embodiments, the reduction of comorbid depression is characterized by a decline in HAM-D value of about nineteen points. In some embodiments, the reduction of comorbid depression is characterized by a decline in HAM-D value of about twenty points.

In some embodiments, after the treatment the patient experiences a substantial reduction of comorbid depression that is characterized by at least a one category change in HAM-D severity classification compared to prior to the treatment. In some embodiments, the reduction of comorbid depression is characterized by a one category change HAM-D severity classification compared to prior to the treatment. In some embodiments, the reduction of comorbid depression is characterized by a two-category change HAM-D severity classification compared to prior to the treatment. In some embodiments, the reduction of comorbid depression is characterized by a three-category change HAM-D severity classification compared to prior to the treatment. In certain embodiments, the reduction of comorbid depression is characterized by remission according to HAM-D value after said treatment (i.e., total HAM-D value of 7 or less).

The Montgomery Åsberg Depression Rating Scale (MADRS) is a depression rating scale consisting of 10 items, each rated 0 to 6. The 10 items represent the core symptoms of depressive illness. The total score of the 10 items ranges from 0 to 60. Decrease in the total score or on individual items indicates improvement (Montgomery S. A. and Åsberg M. A, New Depression Scale Designed to be Sensitive to Change, Br. J. Psychiatry. (1979) April; 134, pages 382-9.).

In some embodiments, after the treatment the patient experiences a substantial reduction of depression that is characterized by at least about a 30% decline in MADRS compared to prior to the treatment. In some embodiments, the reduction of depression is characterized by a decline in MADRS value ranging from about 30% to about 100%, for example, about 30%, about 40%, about 50%, about 60%, about 70%, about 80%, about 90%, and about 100%, compared to prior to the treatment.

In some embodiments, after the treatment the patient experiences a substantial reduction of depression that is characterized by at least a one-point decline in MADRS value compared to prior to the treatment. In some embodiments, the reduction of depression is characterized by a decline in MADRS value ranging from about one point to about five points, for example, about one point, about two points, about three points, about four points, and about five points compared to prior to the treatment. In some embodiments, the reduction of depression is characterized by a decline MADRS value of about two points. In some embodiments, the reduction of depression is characterized by a decline in MADRS value of about three points. In some embodiments, the reduction of depression is characterized by a decline in MADRS value of about four points. In some embodiments, the reduction of depression is characterized by a decline in MADRS value of about five points. In certain embodiments, the reduction of depression is characterized by remission according to MADRS value after said treatment (i.e., MADRS value of 12 or less).

In some embodiments, after the treatment the patient experiences a substantial reduction of comorbid depression that is characterized by at least about a 30% decline in MADRS compared to prior to the treatment. In some embodiments, the reduction of comorbid depression is characterized by a decline in MADRS value ranging from about 30% to about 100%, for example, about 30%, about 40%, about 50%, about 60%, about 70%, about 80%, about 90%, and about 100%, compared to prior to the treatment.

In some embodiments, after the treatment the patient experiences a substantial reduction of comorbid depression that is characterized by at least a one-point decline in MADRS value compared to prior to the treatment. In some embodiments, the reduction of comorbid depression is characterized by a decline in MADRS value ranging from about one point to about five points, for example, about one point, about two points, about three points, about four points, and about five points compared to prior to the treatment. In some embodiments, the reduction of comorbid depression is characterized by a decline MADRS value of about two points. In some embodiments, the reduction of comorbid depression is characterized by a decline in MADRS value of about three points. In some embodiments, the reduction of comorbid depression is characterized by a decline in MADRS value of about four points. In some embodiments, the reduction of comorbid depression is characterized by a decline in MADRS value of about five points. In certain embodiments, the reduction of comorbid depression is characterized by remission according to MADRS value after said treatment (i.e., MADRS value of 12 or less).

The Hamilton Rating Scale for Anxiety (HAM-A) is an anxiety rating scale consisting of 14 items that assess anxious mood, tension, fear, insomnia, intellectual (cognitive) functioning, depressed mood, behavior at interview, somatic (sensory), cardiovascular, respiratory, gastrointestinal, genitourinary, autonomic, and somatic (muscular) symptom (Hamilton, M. The Assessment of Anxiety States by Rating, Br J Med Psychol. (1959); 32 (1), pages 50-5). Each symptom is rated from 0 (absent) to 4 (maximum severity) scale. The total score is used to categorize the severity of anxiety: mild severity (total score less than 17), mild to moderate severity (total score between 18-24), and moderate to severe (total score between 25-30). Total scores range from 0 to 56 with higher scores indicating greater severity.

In some embodiments, after the treatment the patient experiences a substantial reduction of anxiety that is characterized by at least about a 30% decline in total HAM-A value compared to prior to the treatment. In some embodiments, the reduction of anxiety is characterized by a decline in HAM-A value ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of anxiety that is characterized by at least a one-point decline in HAM-A value compared to prior to the treatment. In some embodiments, the reduction of anxiety is characterized by a decline in HAM-A value ranging from about one point to about five points, for example, about one point, about two points, about three points, about four points, and about five points compared to prior to the treatment. In some embodiments, the reduction of anxiety is characterized by a decline HAM-A value of about two points. In some embodiments, the reduction of anxiety is characterized by a decline in HAM-A value of about three points. In some embodiments, the reduction of anxiety is characterized by a decline in HAM-A value of about four points. In some embodiments, the reduction of anxiety is characterized by a decline in HAM-A value of about five points.

In some embodiments, after the treatment the patient experiences a substantial reduction of anxiety that is characterized by at least a one category change in HAM-A severity classification compared to prior to the treatment. In some embodiments, the reduction of anxiety is characterized by a one category change HAM-A severity classification compared to prior to the treatment. In some embodiments, the reduction of anxiety is characterized by a two-category change HAM-A severity classification compared to prior to the treatment. In some embodiments, the reduction of anxiety is characterized by a three-category change HAM-A severity classification compared to prior to the treatment.

In some embodiments, after the treatment the patient experiences a substantial reduction of depression that is characterized by partial remission of the patient's depression. In some embodiments, after the treatment the patient experiences a substantial reduction of MDD that is characterized by partial remission of the patient's depression. In certain embodiments, partial remission of MDD is where the symptoms of the immediately previous major depressive episode are present, but full criteria are not met, or there is a period lasting less than 2 months without any significant symptoms of a major depressive episode following the end of such an episode (i.e., the DSM-5's definition of partial remission).

In some embodiments, after the treatment the patient experiences a substantial reduction of depression that is characterized by full remission of the patient's depression. In some embodiments, after the treatment the patient experiences a substantial reduction of MDD that is characterized by full remission of the patient's depression. In certain embodiments, full remission is where during the past 2 months, no significant signs or symptoms of the disturbance were present (i.e., the DSM-5's definition of full remission).

In some embodiments, after the treatment the patient experiences a substantial reduction of comorbid anxiety that is characterized by at least about a 30% decline in total HAM-A value compared to prior to the treatment. In some embodiments, the reduction of comorbid anxiety is characterized by a decline in HAM-A value ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of comorbid anxiety that is characterized by at least a one-point decline in HAM-A value compared to prior to the treatment. In some embodiments, the reduction of comorbid anxiety is characterized by a decline in HAM-A value ranging from about one point to about five points, for example, about one point, about two points, about three points, about four points, and about five points compared to prior to the treatment. In some embodiments, the reduction of comorbid anxiety is characterized by a decline HAM-A value of about two points. In some embodiments, the reduction of comorbid anxiety is characterized by a decline in HAM-A value of about three points. In some embodiments, the reduction of comorbid anxiety is characterized by a decline in HAM-A value of about four points. In some embodiments, the reduction of comorbid anxiety is characterized by a decline in HAM-A value of about five points.

In some embodiments, after the treatment the patient experiences a substantial reduction of comorbid anxiety that is characterized by at least a one category change in HAM-A severity classification compared to prior to the treatment. In some embodiments, the reduction of comorbid anxiety is characterized by a one category change HAM-A severity classification compared to prior to the treatment. In some embodiments, the reduction of comorbid anxiety is characterized by a two-category change HAM-A severity classification compared to prior to the treatment. In some embodiments, the reduction of comorbid anxiety is characterized by a three-category change HAM-A severity classification compared to prior to the treatment.

In some embodiments, after the treatment the patient experiences a substantial reduction of comorbid depression that is characterized by partial remission of the patient's depression. In certain embodiments, partial remission of comorbid depression is where the symptoms of the immediately previous depressive episode are present, but full criteria are not met, or there is a period lasting less than 2 months without any significant symptoms of a major depressive episode following the end of such an episode (i.e., the DSM-5's definition of partial remission).

In some embodiments, after the treatment the patient experiences a substantial reduction of comorbid depression that is characterized by full remission of the patient's depression. In certain embodiments, full remission is where during the past 2 months, no significant signs or symptoms of the disturbance were present (i.e., the DSM-5's definition of full remission).

The Clinical Global Impression (CGI) (Guy 1976 (Guy W (1976), ECDEU Assessment Manual for Psychopharmacology, Revised. Rockville, MD: US Department of Health, Education and Welfare) consists of three subscales: the CGI-Severity (CGI-S), the CGI-Improvement (CGI-I) and Efficacy Index. The CGI-S assesses the clinician's impression of the patient's current mental illness. A treating clinician categorizes the severity of the patient's current mental illness on a 7-point scale: 1 (normal, not at all ill), 2 (borderline mentally ill), 3 (mildly ill), 4 (moderately ill), 5 (markedly ill), 6 (severely ill), and 7 (among the most extremely ill patients). The CGI-I assesses the participant's improvement (or worsening) from baseline. A treating clinician categorizes the patient's condition relative to Baseline (e.g., prior to administering an antidepressant) on a 7-point scale: 1 (very much improved), 2 (much improved), 3 (minimally improved), 4 (no change), 5 (minimally worse), 6 (much worse), and 7 (very much worse).

In some embodiments, after the treatment the patient experiences a substantial reduction of depression that is characterized by at least a one-point decline in CGI-S value compared to prior to the treatment. In some embodiments, the reduction of depression is characterized by a one-point decline in CGI-S value compared to prior to the treatment. In some embodiments, the reduction of depression is characterized by a two-point decline in CGI-S value compared to prior to the treatment. In some embodiments, the reduction of depression is characterized by a three-point decline in CGI-S value compared to prior to the treatment.

In some embodiments, after the treatment the patient experiences a substantial reduction of depression that is characterized by at least a one-point decline in CGI-I value compared to prior to the treatment. In some embodiments, the reduction of depression is characterized by a one-point decline in CGI-I value compared to prior to the treatment. In some embodiments, the reduction of depression is characterized by a two-point decline in CGI-I value compared to prior to the treatment. In some embodiments, the reduction of depression is characterized by a three-point decline in CGI-I value compared to prior to the treatment.

In some embodiments, after the treatment the patient experiences a substantial reduction of comorbid depression that is characterized by at least a one-point decline in CGI-S value compared to prior to the treatment. In some embodiments, the reduction of depression is characterized by a one-point decline in CGI-S value compared to prior to the treatment. In some embodiments, the reduction of depression is characterized by a two-point decline in CGI-S value compared to prior to the treatment. In some embodiments, the reduction of depression is characterized by a three-point decline in CGI-S value compared to prior to the treatment.

In some embodiments, after the treatment the patient experiences a substantial reduction of comorbid depression that is characterized by at least a one-point decline in CGI-I value compared to prior to the treatment. In some embodiments, the reduction of depression is characterized by a one-point decline in CGI-I value compared to prior to the treatment. In some embodiments, the reduction of depression is characterized by a two-point decline in CGI-I value compared to prior to the treatment. In some embodiments, the reduction of depression is characterized by a three-point decline in CGI-I value compared to prior to the treatment.

The Symptoms of Depression Questionnaire (SDQ) is a 44-item, self-report scale that consists of five subscales: SDQ-1, SDQ-2, SDQ-3, SDQ-4 and SDQ-5. SDQ-1 includes items related to lassitude, mood, and cognitive functioning. SDQ-2 includes items related to anxiety, agitation, irritability, and anger. SDQ-3 includes items related to suicidal ideation. SDQ-4 assesses disruptions in sleep quality. SDQ-5 includes items on changes in appetite and weight. SDQ is used to assess symptom severity across several subtypes of depression (Pedrelli, P., et al., Reliability and Validity of the Symptoms of Depression Questionnaire (SDQ), CNS Spectr. 2014 December; 19(6), pages 535-546.). The items are rated on a 6-point scale. Each item is rated based on a participant's perception of what is normal for the individual (score=2), what is better than normal (score=1), and what is worse than normal (scores=3-6). Total scores range from 0 to 264 with higher scores indicating greater severity.

In some embodiments, after the treatment the patient experiences a substantial reduction of depression that is characterized by at least about a 10% decline in total SDQ scale value compared to prior to the treatment. In some embodiments, the reduction of depression is characterized by a decline in total SDQ scale value ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of depression that is characterized by at least about a 10% decline in at least one subscale value selected from SDQ-1, SDQ-2, SDQ-3, SDQ-4, and SDQ-5 compared to prior to the treatment. In some embodiments, the reduction of depression is characterized by a decline in at least one subscale value selected from SDQ-1, SDQ-2, SDQ-3, SDQ-4 and SDQ-5 value ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of depression that is characterized by at least a one-point decline in Global PSQI (described above) score compared to prior to the treatment. In some embodiments, the reduction of depression is characterized by a one-point decline in Global PSQI score compared to prior to the treatment. In some embodiments, the reduction of depression is characterized by a two-point decline in Global PSQI score compared to prior to the treatment. In some embodiments, the reduction of depression is characterized by a three-point decline in Global PSQI score compared to prior to the treatment.

In some embodiments, after the treatment the patient experiences a substantial reduction of comorbid depression that is characterized by at least about a 10% decline in total SDQ scale value compared to prior to the treatment. In some embodiments, the reduction of depression is characterized by a decline in total SDQ scale value ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of comorbid depression that is characterized by at least about a 10% decline in at least one subscale value selected from SDQ-1, SDQ-2, SDQ-3, SDQ-4, and SDQ-5 compared to prior to the treatment. In some embodiments, the reduction of depression is characterized by a decline in at least one subscale value selected from SDQ-1, SDQ-2, SDQ-3, SDQ-4 and SDQ-5 value ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of comorbid depression that is characterized by at least a one-point decline in Global PSQI (described above) score compared to prior to the treatment. In some embodiments, the reduction of depression is characterized by a one-point decline in Global PSQI score compared to prior to the treatment. In some embodiments, the reduction of depression is characterized by a two-point decline in Global PSQI score compared to prior to the treatment. In some embodiments, the reduction of depression is characterized by a three-point decline in Global PSQI score compared to prior to the treatment.

According to some embodiments, the methods of the present disclosure provide therapeutically effective blood plasma levels of Compound 1 for treating depression, pain, and any of the various tremor conditions disclosed herein. Blood plasma levels of Compound 1 may be expressed using pharmacokinetic parameters that are known to those skilled in the art, such as steady state plasma levels, AUC, Cmax, tₘₐₓ, and Cmin. Throughout the present disclosure pharmacokinetic parameters are described in terms of providing a steady state plasma level of a particular pharmacokinetic parameter (such as steady state plasma Cmax, steady state AUC, etc.). However, the present disclosure contemplates embodiments where the steady state PK parameters that are expressed herein are average values from a patient population (such as a mean value). Thus, the following description of pharmacokinetic parameters describes mean steady state pharmacokinetic parameter values as well as values from an individual patient.

In some embodiments, the present methods provide steady state plasma levels of Compound 1 that correlate to one or more statistically significant therapeutic effects. In certain embodiments, the therapeutically effective steady state plasma levels of Compound 1 provided by the methods of the present disclosure range from about 1 ng/mL to about 500 ng/mL, including about 1 ng/ml, about 5 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL about 80 ng/mL, about 85 ng/mL, about 90 ng/mL, about 95 ng/mL, about 100 ng/mL, about 105 ng/mL, about 110 ng/mL, about 115 ng/mL, about 120 ng/mL, about 125 ng/mL, about 130 ng/mL, about 135 ng/mL, about 140 ng/mL, about 145 ng/mL, about 150 ng/mL, about 155 ng/mL, about 160 ng/mL, about 165 ng/mL, about 170 ng/mL, about 175 ng/mL about 180 ng/mL, about 185 ng/mL, about 190 ng/mL, about 195 ng/mL, about 200 ng/mL, about 205 ng/mL, about 210 ng/mL, about 215 ng/mL, about 220 ng/mL, about 225 ng/mL, about 230 ng/mL, about 235 ng/mL, about 240 ng/mL, about 245 ng/mL, about 250 ng/mL, about 255 ng/mL, about 260 ng/mL, about 265 ng/mL, about 270 ng/mL, about 275 ng/mL, about 280 ng/mL, about 285 ng/mL, about 290 ng/mL, about 295 ng/mL, about 300 ng/mL, about 305 ng/mL, about 310 ng/mL, about 315 ng/mL, about 320 ng/mL, about 325 ng/mL, about 330 ng/mL, about 335 ng/mL, about 340 ng/mL, about 345 ng/mL, about 350 ng/mL, about 355 ng/mL, about 360 ng/mL, about 365 ng/mL, about 370 ng/mL, about 375 ng/mL, about 380 ng/mL, about 385 ng/mL, about 390 ng/mL, about 395 ng/mL, about 400 ng/mL, about 405 ng/mL, about 410 ng/mL, about 415 ng/mL, about 420 ng/mL, about 425 ng/mL, about 430 ng/mL, about 435 ng/mL, about 440 ng/mL, about 445 ng/mL, about 450 ng/mL, about 455 ng/mL, about 460 ng/mL, about 465 ng/mL, about 470 ng/mL, about 475 ng/mL, about 480 ng/mL, about 485 ng/mL, about 490 ng/mL, about 495 ng/mL, and about 500 ng/mL, including all ranges there between. In certain embodiments, the therapeutically effective steady state plasma levels of Compound 1 provided by the methods of the present disclosure range from about 50 ng/ml to 200 ng/ml.

In certain embodiments, the therapeutically effective steady state plasma levels of Compound 1 are provided by administering a daily dose of Compound 1 or a pharmaceutically acceptable salt thereof of about 5 mg. In further embodiments, the therapeutically effective steady state plasma levels of Compound 1 are provided by administering about 5 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 10 mg. In further embodiments, the therapeutically effective steady state plasma levels of Compound 1 are provided by administering about 5 mg of Compound 1 or a pharmaceutically acceptable salt thereof twice a day. In further embodiments, the therapeutically effective steady state plasma levels of Compound 1 are provided by administering about 10 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 15 mg. In further embodiments, the therapeutically effective steady state plasma levels of Compound 1 are provided by administering about 15 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 20 mg. In further embodiments, the therapeutically effective steady state plasma levels of Compound 1 are provided by administering about 10 mg of Compound 1 or a pharmaceutically acceptable salt thereof twice a day. In further embodiments, the therapeutically effective steady state plasma levels of Compound 1 are provided by administering about 20 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 25 mg. In further embodiments, the therapeutically effective steady state plasma levels of Compound 1 are provided by administering about 25 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 30 mg. In further embodiments, the therapeutically effective steady state plasma levels of Compound 1 are provided by administering about 15 mg of Compound 1 or a pharmaceutically acceptable salt thereof twice a day. In further embodiments, the therapeutically effective steady state plasma levels of Compound 1 are provided by administering about 30 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 35 mg. In further embodiments, the therapeutically effective steady state plasma levels of Compound 1 are provided by administering about 35 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 40 mg. In further embodiments, the therapeutically effective steady state plasma levels of Compound 1 are provided by administering about 20 mg of Compound 1 or a pharmaceutically acceptable salt thereof twice a day. In further embodiments, the therapeutically effective steady state plasma levels of Compound 1 are provided by administering about 40 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 45 mg. In further embodiments, the therapeutically effective steady state plasma levels of Compound 1 are provided by administering about 45 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 50 mg. In further embodiments, the therapeutically effective steady state plasma levels of Compound 1 are provided by administering about 25 mg of Compound 1 or a pharmaceutically acceptable salt thereof twice a day. In further embodiments, the therapeutically effective steady state plasma levels of Compound 1 are provided by administering about 50 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 55 mg. In further embodiments, the therapeutically effective steady state plasma levels of Compound 1 are provided by administering about 55 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 60 mg. In further embodiments, the therapeutically effective steady state plasma levels of Compound 1 are provided by administering about 30 mg of Compound 1 or a pharmaceutically acceptable salt thereof twice a day. In further embodiments, the therapeutically effective steady state plasma levels of Compound 1 are provided by administering about 60 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 65 mg. In further embodiments, the therapeutically effective steady state plasma levels of Compound 1 are provided by administering about 65 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 70 mg. In further embodiments, the therapeutically effective steady state plasma levels of Compound 1 are provided by administering about 35 mg of Compound 1 or a pharmaceutically acceptable salt thereof twice a day. In further embodiments, the therapeutically effective steady state plasma levels of Compound 1 are provided by administering about 70 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 75 mg. In further embodiments, the therapeutically effective steady state plasma levels of Compound 1 are provided by administering about 75 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 80 mg. In further embodiments, the therapeutically effective steady state plasma levels of Compound 1 are provided by administering about 40 mg of Compound 1 or a pharmaceutically acceptable salt thereof twice a day. In further embodiments, the therapeutically effective steady state plasma levels of Compound 1 are provided by administering about 80 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 85 mg. In further embodiments, the therapeutically effective steady state plasma levels of Compound 1 are provided by administering about 85 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 90 mg. In further embodiments, the therapeutically effective steady state plasma levels of Compound 1 are provided by administering about 45 mg of Compound 1 or a pharmaceutically acceptable salt thereof twice a day. In further embodiments, the therapeutically effective steady state plasma levels of Compound 1 are provided by administering about 90 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 95 mg. In further embodiments, the therapeutically effective steady state plasma levels of Compound 1 are provided by administering about 95 mg of Compound 1 or a pharmaceutically acceptable salt thereof once a day.

In certain embodiments, the therapeutically effective steady state plasma levels of Compound 1 are provided by administering a daily dose of Compound 1 or a pharmaceutically acceptable salt thereof of about 100 mg. In further embodiments, the therapeutically effective steady state plasma levels of Compound 1 are provided by administering about 50 mg of Compound 1 or a pharmaceutically acceptable salt thereof twice a day. In further embodiments, the therapeutically effective steady state plasma levels of Compound 1 are provided by administering about 100 mg of Compound 1 or a pharmaceutically acceptable salt thereof once a day.

In some embodiments, the present methods provide mean steady state AUC₀₋₂₄ₕ (expressed in terms of ng*hr/mL) levels of Compound 1 that correlate to one or more statistically significant therapeutic effects. In certain embodiments, the therapeutically effective mean steady state AUC₀₋₂₄ₕ levels of Compound 1 provided by the methods of the present disclosure range from about 50 ng*hr/mL to about 2300 ng*hr/mL, including about 50 ng*hr/mL, 100 ng*hr/mL, 150 ng*hr/mL, 200 ng*hr/mL, 250 ng*hr/mL, 300 ng*hr/mL, about 400 ng*hr/mL, about 500 ng*hr/mL, about 600 ng*hr/mL, about 700 ng*hr/mL, about 800 ng*hr/mL, about 900 ng*hr/mL, about 1000 ng*hr/mL, about 1100 ng*hr/mL, about 1200 ng*hr/mL, about 1300 ng*hr/mL, about 1400 ng*hr/mL, about 1500 ng*hr/mL, about 1600 ng*hr/mL, about 1700 ng*hr/mL, about 1800 ng*hr/mL, about 1900 ng*hr/mL, about 2000 ng*hr/mL, about 2100 ng*hr/mL, about 2200 ng*hr/mL and about 2300 ng*hr/mL, including all ranges there between. In certain embodiments, the therapeutically effective mean steady state AUC₀₋₂₄ₕ levels of Compound 1 provided by the methods of the present disclosure range from about 500 ng*hr/mL to about 1000 ng*hr/mL, including about 550 ng*hr/mL, about 600 ng*hr/mL, about 650 ng*hr/mL, about 700 ng*hr/mL, about 750 ng*hr/mL, about 800 ng*hr/mL, about 850 ng*hr/mL and about 900 ng*hr/mL, including all ranges there between. In certain embodiments, the therapeutically effective mean steady state AUC₀₋₂₄ₕ levels of Compound 1 provided by the methods of the present disclosure range from about 600 ng*hr/mL to about 900 ng*hr/mL.

In certain embodiments, the mean steady state AUC₀₋₂₄ₕ of Compound 1 is provided by administering a daily dose of Compound 1 or a pharmaceutically acceptable salt thereof of about 5 mg. In further embodiments, the mean steady state AUC₀₋₂₄ₕ of Compound 1 is provided by administering about 5 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 10 mg. In further embodiments, the mean steady state AUC₀₋₂₄ₕ of Compound 1 is provided by administering about 5 mg of Compound 1 or a pharmaceutically acceptable salt thereof twice a day. In further embodiments, the mean steady state AUC₀₋₂₄ₕ of Compound 1 is provided by administering about 10 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 15 mg. In further embodiments, the mean steady state AUC₀₋₂₄ₕ of Compound 1 is provided by administering about 15 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 20 mg. In further embodiments, the mean steady state AUC₀₋₂₄ₕ of Compound 1 is provided by administering about 10 mg of Compound 1 or a pharmaceutically acceptable salt thereof twice a day. In further embodiments, the mean steady state AUC₀₋₂₄ₕ of Compound 1 is provided by administering about 20 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 25 mg. In further embodiments, the mean steady state AUC₀₋₂₄ₕ of Compound 1 is provided by administering about 25 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 30 mg. In further embodiments, the mean steady state AUC₀₋₂₄ₕ of Compound 1 is provided by administering about 15 mg of Compound 1 or a pharmaceutically acceptable salt thereof twice a day. In further embodiments, the mean steady state AUC₀₋₂₄ₕ of Compound 1 is provided by administering about 30 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 35 mg. In further embodiments, the mean steady state AUC₀₋₂₄ₕ of Compound 1 is provided by administering about 35 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 40 mg. In further embodiments, the mean steady state AUC₀₋₂₄ₕ of Compound 1 is provided by administering about 20 mg of Compound 1 or a pharmaceutically acceptable salt thereof twice a day. In further embodiments, the mean steady state AUC₀₋₂₄ₕ of Compound 1 is provided by administering about 40 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 45 mg. In further embodiments, the mean steady state AUC₀₋₂₄ₕ of Compound 1 is provided by administering about 45 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 50 mg. In further embodiments, the mean steady state AUC₀₋₂₄ₕ of Compound 1 is provided by administering about 25 mg of Compound 1 or a pharmaceutically acceptable salt thereof twice a day. In further embodiments, the mean steady state AUC₀₋₂₄ₕ of Compound 1 is provided by administering about 50 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 55 mg. In further embodiments, the mean steady state AUC₀₋₂₄ₕ of Compound 1 is provided by administering about 55 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 60 mg. In further embodiments, the mean steady state AUC₀₋₂₄ₕ of Compound 1 is provided by administering about 30 mg of Compound 1 or a pharmaceutically acceptable salt thereof twice a day. In further embodiments, the mean steady state AUC₀₋₂₄ₕ of Compound 1 is provided by administering about 60 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 65 mg. In further embodiments, the mean steady state AUC₀₋₂₄ₕ of Compound 1 is provided by administering about 65 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 70 mg. In further embodiments, the mean steady state AUC₀₋₂₄ₕ of Compound 1 is provided by administering about 35 mg of Compound 1 or a pharmaceutically acceptable salt thereof twice a day. In further embodiments, the mean steady state AUC₀₋₂₄ₕ of Compound 1 is provided by administering about 70 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 75 mg. In further embodiments, the mean steady state AUC₀₋₂₄ₕ of Compound 1 is provided by administering about 75 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 80 mg. In further embodiments, the mean steady state AUC₀₋₂₄ₕ of Compound 1 is provided by administering about 40 mg of Compound 1 or a pharmaceutically acceptable salt thereof twice a day. In further embodiments, the mean steady state AUC₀₋₂₄ₕ of Compound 1 is provided by administering about 80 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 85 mg. In further embodiments, the mean steady state AUC₀₋₂₄ₕ of Compound 1 is provided by administering about 85 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 90 mg. In further embodiments, the mean steady state AUC₀₋₂₄ₕ of Compound 1 is provided by administering about 45 mg of Compound 1 or a pharmaceutically acceptable salt thereof twice a day. In further embodiments, the mean steady state AUC₀₋₂₄ₕ of Compound 1 is provided by administering about 90 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 95 mg. In further embodiments, the mean steady state AUC₀₋₂₄ₕ of Compound 1 is provided by administering about 95 mg of Compound 1 or a pharmaceutically acceptable salt thereof once a day.

In certain embodiments, the mean steady state AUC₀₋₂₄ₕ of Compound 1 is provided by administering a daily dose of Compound 1 or a pharmaceutically acceptable salt thereof of about 100 mg. In further embodiments, the mean steady state AUC₀₋₂₄ₕ of Compound 1 is provided by administering about 50 mg of Compound 1 or a pharmaceutically acceptable salt thereof twice a day. In further embodiments, the mean steady state AUC₀₋₂₄ₕ of Compound 1 is provided by administering about 100 mg of Compound 1 or a pharmaceutically acceptable salt thereof once a day.

In some embodiments, the present methods provide an area under the concentration time curve from time zero to infinity AUC_{inf} (expressed in terms of ng*hr/mL) levels of Compound 1 that correlate to one or more statistically significant therapeutic effects. In certain embodiments, the therapeutically effective mean steady state AUC_{inf} levels of Compound 1 provided by the methods of the present disclosure range from about 1000 ng*hr/mL to about 4000 ng*hr/mL, including about 1000 ng*hr/mL, about 1050 ng*hr/mL, about 1100 ng*hr/mL, about 1150 ng*hr/mL, about 1200 ng*hr/mL, about 1250 ng*hr/mL, about 1300 ng*hr/mL, about 1350 ng*hr/mL, about 1400 ng*hr/mL, about 1450 ng*hr/mL, about 1500 ng*hr/mL, about 1550 ng*hr/mL, about 1600 ng*hr/mL, about 1650 ng*hr/mL, about 1700 ng*hr/mL, about 1750 ng*hr/mL, about 1800 ng*hr/mL, about 1850 ng*hr/mL, about 1900 ng*hr/mL, about 1950 ng*hr/mL, about 2000 ng*hr/mL, about 2050 ng*hr/mL, about 2100 ng*hr/mL, about 2150 ng*hr/mL, about 2200 ng*hr/mL, about 2250 ng*hr/mL, about 2300 ng*hr/mL, about 2350 ng*hr/mL, about 2400 ng*hr/mL, about 2450 ng*hr/mL, about 2500 ng*hr/mL, about 2550 ng*hr/mL, about 2600 ng*hr/mL, about 2650 ng*hr/mL, about 2700 ng*hr/mL, about 2750 ng*hr/mL, about 2800 ng*hr/mL, about 2850 ng*hr/mL, about 2900 ng*hr/mL, about 2950 ng*hr/mL, about 3000 ng*hr/mL, about 3050 ng*hr/mL, about 3100 ng*hr/mL, about 3150 ng*hr/mL, about 3200 ng*hr/mL, about 3250 ng*hr/mL, about 3300 ng*hr/mL, about 3350 ng*hr/mL, about 3400 ng*hr/mL, about 3450 ng*hr/mL, about 3500 ng*hr/mL, about 3550 ng*hr/mL, about 3600 ng*hr/mL, about 3650 ng*hr/mL, about 3700 ng*hr/mL, about 3750 ng*hr/mL, about 3800 ng*hr/mL, about 3850 ng*hr/mL, about 3900 ng*hr/mL, about 3950 ng*hr/mL, about 4000 ng*hr/mL, including all ranges there between.

In certain embodiments, the therapeutically effective AUC_{inf} of Compound 1 provided by the methods of the present disclosure ranges from about 2500 ng*hr/mL to about 3500 ng*hr/mL, including about 2500 ng*hr/mL, about 2550 ng*hr/mL, about 2600 ng*hr/mL, about 2650 ng*hr/mL, about 2700 ng*hr/mL, about 2750 ng*hr/mL, about 2800 ng*hr/mL, about 2850 ng*hr/mL, about 2900 ng*hr/mL, about 2950 ng*hr/mL, about 3000 ng*hr/mL, about 3050 ng*hr/mL, about 3100 ng*hr/mL, about 3150 ng*hr/mL, about 3200 ng*hr/mL, about 3250 ng*hr/mL, about 3300 ng*hr/mL, about 3350 ng*hr/mL, about 3400 ng*hr/mL, about 3450 ng*hr/mL, about 3500 ng*hr/mL, including all ranges there between. In certain embodiments, the therapeutically effective mean steady state AUC_{inf} of Compound 1 provided by the methods of the present disclosure ranges from about 1500 ng*hr/mL to about 2500 ng*hr/mL, including about 1500 ng*hr/mL, about 1550 ng*hr/mL, about 1600 ng*hr/mL, about 1650 ng*hr/mL, about 1700 ng*hr/mL, about 1750 ng*hr/mL, about 1800 ng*hr/mL, about 1850 ng*hr/mL, about 1900 ng*hr/mL, about 1950 ng*hr/mL, about 2000 ng*hr/mL, about 2050 ng*hr/mL, about 2100 ng*hr/mL, about 2150 ng*hr/mL, about 2200 ng*hr/mL, about 2250 ng*hr/mL, about 2300 ng*hr/mL, about 2350 ng*hr/mL, about 2400 ng*hr/mL, about 2450 ng*hr/mL, about 2500 ng*hr/mL, including all ranges there between.

In certain embodiments, the AUC_{inf} of Compound 1 is provided by administering a daily dose of Compound 1 or a pharmaceutically acceptable salt thereof of about 5 mg. In further embodiments, the AUC_{inf} levels of Compound 1 is provided by administering about 5 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 10 mg. In further embodiments, the AUC_{inf} levels of Compound 1 is provided by administering about 10 mg of Compound 1 or a pharmaceutically acceptable salt thereof once a day. In further embodiments, the mean steady state AUC_{inf} of Compound 1 is provided by administering about 5 mg of Compound 1 or a pharmaceutically acceptable salt thereof twice a day.

In certain embodiments, the AUC_{inf} of Compound 1 is provided by administering a daily dose of Compound 1 or a pharmaceutically acceptable salt thereof of about 15 mg. In further embodiments, the AUC_{inf} levels of Compound 1 is provided by administering about 15 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 20 mg. In further embodiments, the mean steady state AUC_{inf} of Compound 1 is provided by administering about 10 mg of Compound 1 or a pharmaceutically acceptable salt thereof twice a day. In further embodiments, the mean steady state AUC_{inf} of Compound 1 is provided by administering about 20 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 25 mg. In further embodiments, the AUC_{inf} of Compound 1 is provided by administering about 25 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 30 mg. In further embodiments, the AUC_{inf} of Compound 1 is provided by administering about 15 mg of Compound 1 or a pharmaceutically acceptable salt thereof twice a day. In further embodiments, the AUC_{inf} of Compound 1 is provided by administering about 30 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 35 mg. In further embodiments, the AUC_{inf} of Compound 1 is provided by administering about 35 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 40 mg. In further embodiments, the AUC_{inf} of Compound 1 is provided by administering about 20 mg of Compound 1 or a pharmaceutically acceptable salt thereof twice a day. In further embodiments, the AUC_{inf} of Compound 1 is provided by administering about 40 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 45 mg. In further embodiments, the AUC_{inf} of Compound 1 is provided by administering about 45 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 50 mg. In further embodiments, the AUC_{inf} of Compound 1 is provided by administering about 25 mg of Compound 1 or a pharmaceutically acceptable salt thereof twice a day. In further embodiments, the AUC_{inf} of Compound 1 is provided by administering about 50 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 55 mg. In further embodiments, the AUC_{inf} of Compound 1 is provided by administering about 55 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 60 mg. In further embodiments, the AUC_{inf} of Compound 1 is provided by administering about 30 mg of Compound 1 or a pharmaceutically acceptable salt thereof twice a day. In further embodiments, the AUC_{inf} of Compound 1 is provided by administering about 60 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 65 mg. In further embodiments, the AUC_{inf} of Compound 1 is provided by administering about 65 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 70 mg. In further embodiments, the AUC_{inf} of Compound 1 is provided by administering about 35 mg of Compound 1 or a pharmaceutically acceptable salt thereof twice a day. In further embodiments, the AUC_{inf} of Compound 1 is provided by administering about 70 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 75 mg. In further embodiments, the AUC_{inf} of Compound 1 is provided by administering about 75 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 80 mg. In further embodiments, the AUC_{inf} of Compound 1 is provided by administering about 40 mg of Compound 1 or a pharmaceutically acceptable salt thereof twice a day. In further embodiments, the AUC_{inf} of Compound 1 is provided by administering about 80 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 85 mg. In further embodiments, the AUC_{inf} of Compound 1 is provided by administering about 85 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 90 mg. In further embodiments, the AUC_{inf} of Compound 1 is provided by administering about 45 mg of Compound 1 or a pharmaceutically acceptable salt thereof twice a day. In further embodiments, the AUC_{inf} of Compound 1 is provided by administering about 90 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 95 mg. In further embodiments, the AUC_{inf} of Compound 1 is provided by administering about 95 mg of Compound 1 or a pharmaceutically acceptable salt thereof once a day.

In certain embodiments, the AUC_{inf} of Compound 1 is provided by administering a daily dose of Compound 1 or a pharmaceutically acceptable salt thereof of about 100 mg. In further embodiments, the AUC_{inf} of Compound 1 is provided by administering about 50 mg of Compound 1 or a pharmaceutically acceptable salt thereof twice a day. In further embodiments, the AUC_{inf} of Compound 1 is provided by administering about 100 mg of Compound 1 or a pharmaceutically acceptable salt thereof once a day.

In some embodiments, the present methods provide steady state plasma Cmax levels (or mean steady state plasma Cmax levels) of Compound 1 that correlate to one or more statistically significant therapeutic effects. In certain embodiments, the therapeutically effective steady state plasma Cmax levels (or mean steady state plasma Cmax levels) of Compound 1 provided by the methods of the present disclosure range from about 5 ng/mL to about 500 ng/mL, including about 5 ng/mL, 10 ng/mL, 20 ng/mL, 30 ng/mL, 40 ng/mL, 50 ng/mL, 60 ng/mL, about 70 ng/mL, about 80 ng/mL, about 90 ng/mL, about 100 ng/mL, about 110 ng/mL, about 120 ng/mL, about 130 ng/mL, about 140 ng/mL, about 150 ng/mL, about 160 ng/mL, about 170 ng/mL about 180 ng/mL, about 190 ng/mL, about 200 ng/mL, about 210 ng/mL, about 220 ng/mL, about 230 ng/mL, about 240 ng/mL, about 250 ng/mL, about 260 ng/mL, about 270 ng/mL about 280 ng/mL, about 290 ng/mL, about 300 ng/mL, about 310 ng/mL, about 320 ng/mL, about 330 ng/mL, about 340 ng/mL, about 350 ng/mL, about 360 ng/mL, about 370 ng/mL about 380 ng/mL, about 390 ng/mL, about 400 ng/mL, about 410 ng/mL, about 420 ng/mL, about 430 ng/mL, about 440 ng/mL, about 150 ng/mL, about 460 ng/mL, about 470 ng/mL about 480 ng/mL, about 490 ng/mL, about 500 ng/mL, about 510 ng/mL about 520 ng/mL, about 530 ng/mL, about 540 ng/mL, about 550 ng/mL, about 560 ng/mL, about 570 ng/mL about 580 ng/mL, about 590 ng/mL and about 600 ng/mL, including all ranges there between. In some embodiments, the therapeutically effective steady state plasma Cmax levels (or mean steady state plasma Cmax levels) of Compound 1 provided by the methods of the present disclosure are from about 100 ng/mL to about 275 ng/mL, including about 110 ng/mL, about 120 ng/mL, about 130 ng/mL, about 140 ng/mL, about 150 ng/mL, about 160 ng/mL, about 170 ng/mL about 180 ng/mL, about 190 ng/mL, about 200 ng/mL, about 210 ng/mL, about 220 ng/mL, about 230 ng/mL, about 240 ng/mL, about 250 ng/mL, about 260 ng/mL, about 270 ng/mL, including all ranges there between. In some embodiments, the therapeutically effective steady state plasma Cmax levels (or mean steady state plasma Cmax levels) of Compound 1 provided by the methods of the present disclosure are from about 125 ng/mL to about 250 ng/mL.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 5 mg. In further embodiments, the therapeutically effective steady state plasma Cmax levels (or mean steady state plasma Cmax levels) of Compound 1 is provided by administering about 5 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 10 mg. In further embodiments, the therapeutically effective steady state plasma Cmax levels (or mean steady state plasma Cmax levels) of Compound 1 is provided by administering about 10 mg of Compound 1 or a pharmaceutically acceptable salt thereof once a day. In further embodiments, the therapeutically effective steady state plasma Cmax levels (or mean steady state plasma Cmax levels) of Compound 1 is provided by administering about 5 mg of Compound 1 or a pharmaceutically acceptable salt thereof twice a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 15 mg. In further embodiments, the therapeutically effective steady state plasma Cmax levels (or mean steady state plasma Cmax levels) of Compound 1 is provided by administering about 15 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 20 mg. In further embodiments, the therapeutically effective steady state plasma Cmax levels (or mean steady state plasma Cmax levels) of Compound 1 is provided by administering about 10 mg of Compound 1 or a pharmaceutically acceptable salt thereof twice a day. In further embodiments, the therapeutically effective steady state Cmax plasma levels (or mean steady state plasma Cmax levels) of Compound 1 is provided by administering about 20 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 25 mg. In further embodiments, the therapeutically effective steady state plasma Cmax levels (or mean steady state plasma Cmax levels) of Compound 1 is provided by administering about 25 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 30 mg. In further embodiments, the therapeutically effective steady state plasma Cmax levels (or mean steady state plasma Cmax levels) of Compound 1 is provided by administering about 15 mg of Compound 1 or a pharmaceutically acceptable salt thereof twice a day. In further embodiments, the therapeutically effective steady state Cmax plasma levels (or mean steady state plasma Cmax levels) of Compound 1 is provided by administering about 30 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 35 mg. In further embodiments, the therapeutically effective steady state plasma Cmax levels (or mean steady state plasma Cmax levels) of Compound 1 is provided by administering about 35 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 40 mg. In further embodiments, the therapeutically effective steady state plasma Cmax levels (or mean steady state plasma Cmax levels) of Compound 1 is provided by administering about 20 mg of Compound 1 or a pharmaceutically acceptable salt thereof twice a day. In further embodiments, the therapeutically effective steady state Cmax plasma levels (or mean steady state plasma Cmax levels) of Compound 1 is provided by administering about 40 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 45 mg. In further embodiments, the therapeutically effective steady state plasma Cmax levels (or mean steady state plasma Cmax levels) of Compound 1 is provided by administering about 45 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 50 mg. In further embodiments, the therapeutically effective steady state plasma Cmax levels (or mean steady state plasma Cmax levels) of Compound 1 is provided by administering about 25 mg of Compound 1 or a pharmaceutically acceptable salt thereof twice a day. In further embodiments, the therapeutically effective steady state Cmax plasma levels (or mean steady state plasma Cmax levels) of Compound 1 is provided by administering about 50 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 55 mg. In further embodiments, the therapeutically effective steady state plasma Cmax levels (or mean steady state plasma Cmax levels) of Compound 1 is provided by administering about 55 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 60 mg. In further embodiments, the therapeutically effective steady state plasma Cmax levels (or mean steady state plasma Cmax levels) of Compound 1 is provided by administering about 30 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 60 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 65 mg. In further embodiments, the therapeutically effective steady state plasma Cmax levels (or mean steady state plasma Cmax levels) of Compound 1 is provided by administering about 65 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 70 mg. In further embodiments, the therapeutically effective steady state plasma Cmax levels (or mean steady state plasma Cmax levels) of Compound 1 is provided by administering about 35 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 70 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 75 mg. In further embodiments, the therapeutically effective steady state plasma Cmax levels (or mean steady state plasma Cmax levels) of Compound 1 is provided by administering about 75 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 80 mg. In further embodiments, the therapeutically effective steady state plasma Cmax levels (or mean steady state plasma Cmax levels) of Compound 1 is provided by administering about 40 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 80 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 85 mg. In further embodiments, the therapeutically effective steady state plasma Cmax levels (or mean steady state plasma Cmax levels) of Compound 1 is provided by administering about 85 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 90 mg. In further embodiments, the therapeutically effective steady state plasma Cmax levels (or mean steady state plasma Cmax levels) of Compound 1 is provided by administering about 45 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 90 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 95 mg. In further embodiments, the therapeutically effective steady state plasma Cmax levels (or mean steady state plasma Cmax levels) of Compound 1 is provided by administering about 95 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 100 mg. In further embodiments, the therapeutically effective steady state plasma Cmax levels (or mean steady state plasma Cmax levels) of Compound 1 is provided by administering about 50 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 100 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 105 mg. In further embodiments, the therapeutically effective steady state plasma Cmax levels (or mean steady state plasma Cmax levels) of Compound 1 is provided by administering about 105 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 110 mg. In further embodiments, the therapeutically effective steady state plasma Cmax levels (or mean steady state plasma Cmax levels) of Compound 1 is provided by administering about 55 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 110 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 115 mg. In further embodiments, the therapeutically effective steady state plasma Cmax levels (or mean steady state plasma Cmax levels) of Compound 1 is provided by administering about 115 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof of about 120 mg. In further embodiments, the therapeutically effective steady state plasma Cmax levels (or mean steady state plasma Cmax levels) of Compound 1 is provided by administering about 60 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 120 mg of Compound 1 or a pharmaceutically acceptable salt thereof once a day.

In some embodiments, the present methods provide steady state plasma Cmax levels (or mean steady state plasma Cmax levels) of Compound 1 that do not exceed 500 ng/mL. In certain embodiments, the therapeutically effective steady state plasma Cmax levels of Compound 1 provided by the methods of the present disclosure do not exceed about 500 ng/mL, including less than about 500 ng/mL, less than about 475 ng/mL, less than about 450 ng/mL, than about 425 ng/mL, less than about 400 ng/mL, less than about 375 ng/mL, than about 350 ng/mL, less than about 325 ng/mL, and less than about 300 ng/mL.

In some embodiments, administration of Compound 1 or a pharmaceutically acceptable salt thereof in the evening provides steady state plasma Cmax levels (or mean steady state plasma Cmax levels) of Compound 1 that are reduced as compared to administration of Compound 1 or a pharmaceutically acceptable salt thereof in the morning. In some embodiments, Cmax after evening dosing is reduced by at least about 25%, for example, about 25%, about 30%, about 35%, about 40%, about 45%, about 50%, about 55%, about 60%, about 65%, about 70%, or about 75% compared to Cmax after morning dosing. In some embodiments, Cmax after evening dosing is less than about 327 ng/mL, for example, about 327 ng/mL, about 300 ng/mL, about 275 ng/mL, about 250 ng/mL, about 225 ng/mL, about 200 ng/mL, about 175 ng/mL, or about 150 ng/mL.

In some embodiments, administration of Compound 1 or a pharmaceutically acceptable salt thereof in the evening provides a tₘₐₓ of Compound 1 that are increased as compared to administration of Compound 1 or a pharmaceutically acceptable salt thereof in the morning. In some embodiments, tₘₐₓ after evening dosing is increased by at least about 50%, for example, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, or about 500% compared to tₘₐₓ after morning dosing. In some embodiments, Cmax after evening dosing is greater than about 1 hour, for example, about 1.25 hours, about 1.5 hours, about 1.75 hours, about 2 hours, about 2.25 hours, about 2.5 hours, about 2.75 hours, about 3 hours, about 3.5 hours, about 3.75 hours, about 4 hours, or more.

In some embodiments, administration of Compound 1 or a pharmaceutically acceptable salt thereof in the evening provides an AUC_{inf} that is substantially similar to the AUC_{inf} provided by administration of Compound 1 or a pharmaceutically acceptable salt thereof in the morning. In some embodiments, an AUC_{inf} provided by evening dosing that is substantially similar to an AUC_{inf} provided by morning dosing is within 15% of the AUC_{inf} provided by morning dosing, for example, within about 1%, about 2%, about 3%, about 4%, about 5%, about 6%, about 7%, about 8%, about 9%, about 10%, about 11%, about 12%, about 13%, about 14%, or about 15 of the AUC_{inf} provided by morning dosing. For example, an AUC_{inf} of 2510 ng*hr/mL provided by evening dosing is substantially similar to an AUC_{inf} of 2610 ng*hr/mL provided by morning dosing. In some embodiments, an AUC_{inf} provided by evening dosing that is substantially similar to an AUC_{inf} provided by morning dosing is the same as the AUC_{inf} provided by morning dosing.

In some embodiments of the present methods, reducing the symptoms of depression, anxiety, insomnia, essential, tremor, and/or adjustment disorder comprises administration of Compound 1 or a pharmaceutically acceptable salt thereof to a subject in need thereof.

In some embodiments, administration of Compound 1 or a pharmaceutically acceptable salt thereof to a subject in need thereof provides an increase in qEEG beta-band frequency compared to baseline (i.e., prior to administering Compound 1 or a pharmaceutically acceptable salt thereof). In some embodiments, the increase in qEEG beta-band frequency is from about 1.1-times to about 4-times compared to baseline (i.e., prior to administering Compound 1 or a pharmaceutically acceptable salt thereof). In some embodiments, the administration of Compound 1 or a pharmaceutically acceptable salt thereof provides a plasma concentration from about 1 ng/mL to about 500 ng/mL and an about 1.1-times to about 4-times increase in qEEG beta-band frequency compared to baseline. In some embodiments, the increase in qEEG beta-band frequency occurs 0.5 to 5 h after dosing (e.g., about 0.5, 1, 1.5, 2, 2.5, 3, 3.5, 4, 4.5, or 5 hours after dosing). In some embodiments, the increase in beta-band frequency is associated with a reduction of side effects selected from the group consisting of somnolence, sedation, dizziness, lightheadedness, fatigue, euphoria, feeling drunk, feeling abnormal, headache, cognitive disorder, disturbance of attention, memory impairment, slowed cognition, decreased alertness, constipation, and diarrhea. In some embodiments, administration of Compound 1 or a pharmaceutically acceptable salt thereof is nightly administration.

In some embodiments, administration of Compound 1 or a pharmaceutically acceptable salt thereof to a subject in need thereof provides an increase in qEEG alpha-band frequency compared to baseline (i.e., prior to administering Compound 1 or a pharmaceutically acceptable salt thereof). In some embodiments, the increase in qEEG alpha-band frequency is from about 1.1-times to about 4-times compared to baseline (i.e., prior to administering Compound 1 or a pharmaceutically acceptable salt thereof). In some embodiments, the administration of Compound 1 or a pharmaceutically acceptable salt thereof provides a plasma concentration from about 1 ng/mL to about 500 ng/mL and an about 1.1-times to about 4-times increase in qEEG alpha-band frequency compared to baseline. In some embodiments, the increase in qEEG alpha-band frequency occurs 0.5 to 5 h after dosing (e.g., about 0.5, 1, 1.5, 2, 2.5, 3, 3.5, 4, 4.5, or 5 hours after dosing). In some embodiments, the increase in alpha-band frequency is associated with a reduction of side effects selected from the group consisting of somnolence, sedation, dizziness, lightheadedness, fatigue, euphoria, feeling drunk, feeling abnormal, headache, cognitive disorder, disturbance of attention, memory impairment, slowed cognition, decreased alertness, constipation, and diarrhea. In some embodiments, administration of Compound 1 or a pharmaceutically acceptable salt thereof is nightly administration.

In some embodiments, administration of Compound 1 or a pharmaceutically acceptable salt thereof to a subject in need thereof provides an increase in qEEG alpha- and/or beta-band frequency compared to baseline (i.e., prior to administering Compound 1 or a pharmaceutically acceptable salt thereof). In some embodiments, the increase in qEEG alpha- and/or beta-band frequency is from about 1.1-times to about 4-times compared to baseline (i.e., prior to administering Compound 1 or a pharmaceutically acceptable salt thereof). In some embodiments, the administration of Compound 1 or a pharmaceutically acceptable salt thereof provides a plasma concentration from about 1 ng/mL to about 500 mg/mL and an about 1.1-times to about 4-times increase in qEEG alpha- and/or beta-band frequency compared to baseline. In some embodiments, the increase in qEEG alpha- and/or beta-band frequency occurs 0.5 to 5 h after dosing (e.g., about 0.5, 1, 1.5, 2, 2.5, 3, 3.5, 4, 4.5, or 5 hours after dosing). In some embodiments, the increase in alpha- and/or beta-band frequency is associated with a reduction of side effects selected from the group consisting of somnolence, sedation, dizziness, lightheadedness, fatigue, euphoria, feeling drunk, feeling abnormal, headache, cognitive disorder, disturbance of attention, memory impairment, slowed cognition, decreased alertness, constipation, and diarrhea. In some embodiments, administration of Compound 1 or a pharmaceutically acceptable salt thereof is nightly administration.

In some embodiments, the subject in need thereof is a patient that demonstrates a response in the reduction of symptoms related to depression, insomnia, essential tremor, and/or adjustment disorder.

### Methods of Treating Substance Abuse Use Disorder

In some embodiments, the present disclosure provides methods of treating a substance abuse disorder (such as opioid use disorder) comprising administering an effective amount of Compound 1 or a pharmaceutically acceptable salt thereof. In some embodiments, the present methods employ Compound 1, or a pharmaceutically acceptable salt thereof, as the sole active ingredient used to treat a substance abuse disorder. In some embodiments, the present methods employ Compound 1, or a pharmaceutically acceptable salt thereof, in conjunction with one or more active ingredients used to treat a substance abuse disorder. In some embodiments, Compound 1 or a pharmaceutically acceptable salt thereof is administered in combination with an additional active ingredient used to treat a substance abuse disorder, e.g., co-formulated or administered separately.

Opioid use disorder includes signs and symptoms that reflect compulsive, prolonged self-administration of opioid substances that are used for no legitimate medical purpose or, if another medical condition is present that requires opioid treatment, that are used in doses greatly in excess of the amount needed for that medical condition. For example, an individual prescribed analgesic opioids for pain relief at adequate dosing will use significantly more than prescribed and not only because of persistent pain.

Substance abuse disorder, including cocaine, alcohol, and opioids has been associated with the dopamine reward pathways (Ross, S. & Peselow, E. The Neurobiology of Addictive Disorders. Clin Neuropharmacol 32, 269-276 (2009). The neurotransmitter GABA suppresses striatal dopamine release and blunts cocaine-induced increases in extracellular dopamine in the striatum and nucleus accumbens in animals (Dewey, S. et al. GABAergic inhibition of endogenous dopamine release measured in vivo with 11C-raclopride and positron emission tomography. J Neurosci 12, 3773-3780 (1992). Progesterone treatment has been shown to decrease craving to cocaine in clinical studies, potentially due to the GABAergic effect GABA-A positive allosteric modulator neuroactive steroids synthesized from progesterone (Sinha, R. et al. Sex steroid hormones, stress response, and drug craving in cocaine-dependent women: Implications for relapse susceptibility. Exp Clin Psychopharm 15, 445 (2007).

Stress may be a factor in the development of substance use disorders and in perpetuating the cycle of drug use, abstinence, and relapse in addicted individuals.

Progesterone treatment has been shown to decrease craving to cocaine in clinical studies, potentially due to the GABAergic effect GABA-A positive allosteric modulator neuroactive steroids synthesized from progesterone.

In some embodiments, the present disclosure provides methods of treating opioid use disorder comprising administering an effective amount of Compound 1 or a pharmaceutically acceptable salt thereof. In some embodiments, the Compound 1 or a pharmaceutically acceptable salt thereof is administered as a monotherapy. In some embodiments, the Compound 1 or a pharmaceutically acceptable salt thereof is administered as an adjunctive to the patient's existing therapy (e.g., the current standard of care). In certain embodiments, the Compound 1 or a pharmaceutically acceptable salt thereof is administered as an adjunctive to methadone. In certain embodiments, the Compound 1 or a pharmaceutically acceptable salt thereof is administered as an adjunctive to buprenorphine.

In some embodiments, after said treatment the patient experiences a substantial reduction of opioid use disorder that is characterized by an abstinence to opioid use during the period of Compound 1 or a pharmaceutically acceptable salt thereof administration. As used herein “abstinence to opioid use” means a negative urine drug test and no self-reported opioid use on the timeline follow-back (TLFB) survey during the period of Compound 1 or a pharmaceutically acceptable salt thereof administration. TLFB survey use calendars and daily recall of substance use on specific days to record quantity or frequency of opioid use. Omission of any of these criteria resulted in failure to confirm abstinence for the week.

In some embodiments, after said treatment the patient experiences a substantial reduction of opioid use disorder that is characterized by a statistically significant decrease in the percentage of opioid-free weeks in an Compound 1 or a pharmaceutically acceptable salt thereof treated group compared to a placebo treated group during the period of Compound 1 or a pharmaceutically acceptable salt thereof administration (i.e., there is a significant statistical difference between the percentage of opioid-free weeks of Compound 1 or a pharmaceutically acceptable salt thereof treatment relative to placebo treatment).

In some embodiments, after said treatment the patient experiences a substantial reduction of opioid use disorder that is characterized by substantial improvement is demonstrated by craving assessment—weekly self-report visual analogue scale (VAS) of need for opioids (scale 0-100, 0=not at all; 100=very much so). Response defined by significant statistical difference in the mean change in VAS score from baseline of treatment group relative to placebo (Krupitsky, E. et al. Injectable extended-release naltrexone for opioid dependence: a double-blind, placebo-controlled, multicentre randomised trial. Lancet 377, 1506-1513 (2006).

In some embodiments, after said treatment the patient experiences a substantial reduction of opioid use disorder that is characterized by a statistically significant change in retention assessment compared to a placebo treated group. As used herein, “retention assessment” means the number of days of retention on either cognitive behavioral therapy or pharmacotherapy by TLFB during the period of Compound 1 or a pharmaceutically acceptable salt thereof administration.

In certain embodiments, after said treatment the patient experiences a statistically significant change in retention assessment that is characterized by significant statistical difference in the mean change in number of days of retention in Compound 1 or a pharmaceutically acceptable salt thereof treatment group relative to placebo.

In some embodiments, the present disclosure provides methods of treating cocaine use disorder comprising administering an effective amount of Compound 1 or a pharmaceutically acceptable salt thereof. In some embodiments, the Compound 1 or a pharmaceutically acceptable salt thereof is administered as a monotherapy. In some embodiments, the Compound 1 or a pharmaceutically acceptable salt thereof is administered as an adjunctive to the current standard of care. In some embodiment, the Compound 1 or a pharmaceutically acceptable salt thereof is administered as an adjunctive to buprenorphine. In some embodiments, the Compound 1 or a pharmaceutically acceptable salt thereof is administered as an adjunctive to buprenorphine and naloxone. In some embodiments, the Compound 1 or a pharmaceutically acceptable salt thereof is administered as an adjunctive to naltrexone. In some embodiments, the Compound 1 or a pharmaceutically acceptable salt thereof administered as an adjunctive to lofexidine.

In some embodiments, the present disclosure provides methods of treating alcohol use disorder comprising administering an effective amount of Compound 1 or a pharmaceutically acceptable salt thereof. In some embodiments, the Compound 1 or a pharmaceutically acceptable salt thereof is administered as a monotherapy. In some embodiments, the Compound 1 or a pharmaceutically acceptable salt thereof is administered as an adjunctive to the current standard of care. In some embodiments, the Compound 1 or a pharmaceutically acceptable salt thereof is administered as an adjunctive to a benzodiazepine.

In some embodiments, the present disclosure provides methods of treating benzodiazepine use disorder comprising administering an effective amount of Compound 1 or a pharmaceutically acceptable salt thereof. In some embodiments, the Compound 1 or a pharmaceutically acceptable salt thereof is administered as a monotherapy. In some embodiments, the Compound 1 or a pharmaceutically acceptable salt thereof is administered as an adjunctive to the current standard of care. In some embodiments, the Compound 1 or a pharmaceutically acceptable salt thereof is administered as an adjunctive to medically supervised withdrawal (detoxification). In some embodiments, the Compound 1 or a pharmaceutically acceptable salt thereof is administered as an adjunctive to residential rehabilitation treatment. In some embodiments, the Compound 1 or a pharmaceutically acceptable salt thereof is administered as an adjunctive to mutual help groups. In some embodiments, the Compound 1 or a pharmaceutically acceptable salt thereof is administered as an adjunctive to outpatient substance use disorder services (e.g., counseling or medication for addiction).

### Methods of Treating Motor Disorders Such as Tremor, Including Essential Tremor

In some embodiments, the present disclosure provides methods of treating motor disorders, including any of the tremors disclosed herein, particularly essential tremor comprising administering an effective amount of Compound 1 or a pharmaceutically acceptable salt thereof. In some embodiments, the present methods employ Compound 1, or a pharmaceutically acceptable salt thereof, as the sole active ingredient used to treat the motor disorder (e.g., essential tremor). In some embodiments, the present methods employ Compound 1, or a pharmaceutically acceptable salt thereof, in conjunction with one or more active ingredients used to treat the motor disorder. In some embodiments, Compound 1 or a pharmaceutically acceptable salt thereof is administered in combination with an additional active ingredient used to treat a motor disorder, e.g., co-formulated or administered separately.

In some embodiments, the present disclosure provides methods of treating a motor disorder such as essential tremor comprising administering an effective amount of Compound 1 or a pharmaceutically acceptable salt thereof. In some embodiments, the Compound 1 or a pharmaceutically acceptable salt thereof is administered as a monotherapy. In some embodiments, the Compound 1 or a pharmaceutically acceptable salt thereof is administered as an adjunctive to the patient's existing therapy (e.g., the current standard of care). In certain embodiments, the Compound 1 or a pharmaceutically acceptable salt thereof is administered in combination with a T-type calcium channel blocker, for example Compound 2 or pharmaceutically acceptable salts thereof.

In some embodiments, after said treatment the patient experiences a substantial reduction of the symptoms of the motor disorder (e.g., essential tremor) that is characterized by reduction of the frequency or severity of tremors during the period of Compound 1 or a pharmaceutically acceptable salt thereof administration.

In some embodiments, after said treatment the patient experiences a statistically significant decrease in the frequency or severity of tremors in a Compound 1 or a pharmaceutically acceptable salt thereof treated group compared to a placebo treated group during the period of Compound 1 or a pharmaceutically acceptable salt thereof administration.

### EXAMPLES

The present disclosure is further illustrated by reference to the following Examples. However, it is noted that these Examples, like the embodiments described above, are illustrative and are not to be construed as restricting the scope of the disclosure in any way.

### Example 1

Healthy subjects aged 18 to 55 years were treated with an oral suspension of Compound 1 to study the safety, tolerability, pharmacokinetics and pharmacodynamics of Compound 1 in healthy subjects. Dose and dose frequencies were evaluated in order to select a regimen that is suitable for subjects with MDD. From the results of the study, oral Compound 1 will be assessed for its potential to reduce the symptoms of MDD in a dose-dependent manner.

### Study Design

The study was a randomized, double-blind, placebo-controlled multiple escalating dose study comprised of 3 cohorts that each received an oral suspension. Each cohort consisted of two groups: one group treated with Compound 1 and another treated with placebo. In each cohort, the ratio of Compound 1-treated subjects to placebo-treated subjects was 3:1.

The Compound 1-treated subjects of Cohort 1 were treated with 15.0 mg of Compound 1 once per day (QD). The Compound 1-treated subjects of Cohort 2 were treated with 30.0 mg of Compound 1 QD. The Compound 1-treated subjects of Cohort 3 were treated with 60.0 mg of Compound 1 QD.

A Food Effect Cohort (Cohort 4) was conducted to assess the effect of food on the PK profile of a single dose of Compound 1 when administered to healthy subjects. The subjects of Cohort 4 were treated with 30 mg of Compound 1 QD.

### Dosing:

Patients in each cohort were treated with Compound 1 for 14 consecutive days, unless dosing was halted by the Safety Review Committee (SRC). The dosing of subjects in each of the cohorts was staggered with the decision to dose escalate based on SRC review of a minimum of 14 days of observation of safety and tolerability data and review of the available plasma PK data from the preceding cohort(s). Thus, dose escalation was predicated on tolerability of the prior cohorts.

Compound 1 was administered under fasted conditions (no food or drink, except water, for at least 10 hours prior to dosing). Immediately after administration of Compound 1, the subject was be administered 240 mL water. No additional fluid intake was allowed until 1 hour after Compound 1 administration.

Cohort 1 subjects received a single 15.0 mg dose of a Compound 1 suspension on the morning of Days 1-14. Cohort 2 subjects received a single 30.0 mg dose of a Compound 1 suspension on the morning of Days 1-14. Cohort 3 subjects received a single 60.0 mg dose of a Compound 1 suspension on the morning of Days 1-14. For all cohorts, the last treatment was administered on the morning of Day 14.

Cohort 4 subjects received a single 30 mg dose of a Compound 1 suspension on Days 1 and 5. The Day 1 dose was administered after a minimum of 10 hour fasting. No additional fluid intake was allowed until 1 hour after drug administration. A standard meal was given at least 4 hours post-dose. The Day 5 dose was administered following a high-fat, high calorie meal. Participants remained at the clinical site for a total of 8 days to complete pharmacokinetic sampling after the second dose.

Blood and urine were obtained during each treatment period at designated times for pharmacokinetic and other analyses (see below). Standard safety assessments were measured during each treatment period.

### Pharmacokinetic (PK) Assessments

PK parameters (e.g., Cₘₐₓ, Tₘₐₓ, T_{1/2}, AUC, etc.) for healthy patients in each cohort were compared to assess the suitability of Compound 1 suspension for the treatment of MDD. Data were obtained from the blood plasma samples collected from each cohort according to the schedule provided.

Plasma samples were analyzed to determine Compound 1 concentrations using a validated assay method. Pharmacokinetic variables (including but not limited to Cₘₐₓ, Tₘₐₓ and AUC₍₀₋ₗₐₛₜ₎) were calculated using non-compartmental analysis. PK parameters for Compound 1 were derived from the plasma concentration data using non-compartmental analysis with Phoenix™ WinNonlin® v 8.0 (Pharsight Corporation, USA).

### Protocol:

Blood (Cohorts 1-3): For each cohort, blood samples were collected on Days 1, 2, 3, 4, 5, 6 and 14 at the following time points: Day 1 at pre-dose (0 hour), 0.25, 0.5, 0.75, 1, 1.5, 2, 2.5, 3, 4, 6, 8, 12, 16 h post-dose; Day 2 at pre-dose (24 hours), Day 3 at pre-dose (48 hours), Day 4 at pre-dose (72 hours), Day 5 at pre-dose (96 hours), Day 6 at pre-dose (120 hours); Day 14 at pre-dose 0.25, 0.5, 0.75, 1, 1.5, 2, 2.5, 3, 4, 6, 8, 12, 16, 24, 36, 48, and 72 hours. Trough level blood samples were collected on Days 2, 3, 4, 5, 6 and 14, prior to the morning dose administrations.

The following PK parameters were calculated based on the plasma concentrations of Compound 1: maximum observed concentration (Cmax) on Day 1 and at steady state on Day 15 (Cmax,SS), Time of Cmax (Tmax) and Cmax,SS (Tmax,SS), area under the concentration-time curve through the dosing interval on Day 1 and 15 (AUCtau and AUCSS), total clearance at steady state, measured on Day 15 (CLSS), and volume of distribution at steady state, measured on Day 15 (VSS).

Urine (Cohorts 1-3): Urine was collected/pooled at the following collection windows: Day −1 (6 hours) and at Day 14: (0 to 6 hours), (6 to 12 hours), (12 to 24 hours), and (24-48 hours). Urine samples were analyzed to determine Compound 1 concentrations using a validated assay method. Pooling of urine across patients may be allowed if volumes are not sufficient to allow individual determination.

The following PK parameters were calculated based on the urine concentrations of Compound 1: absolute and cumulative amount of Compound 1 excreted in urine and renal clearance (CLR).

Blood (Cohort 4): Serial blood samples were collected relative to the dosing of Compound 1 at the following time points on both Day 1 and Day 5: Pre-dose (0 hours), 0.25, 0.50, 0.75, 1.00, 1.50, 2.00, 2.50, 3.00, 4.00, 6.00, 8.00, 12.00, 16.00, 24.00, 36.00, 48.00 and 72.00 hours post-dose (±2 min). Urine (Cohort 4): No urine analysis was conducted in Cohort 4.

### Pharmacodynamic Assessment

Pharmacodynamic (PD) effects of first dose and steady state Compound 1 concentrations on wake electroencephalograms (EEGs) were studied. Standard 16 channel continuous EEGs were obtained at the following time points: Day-1, Day 1 (1 hour after dosing), and Day 14 (1 hour after dosing).

### Safety Assessments/Monitoring

Adverse events (AEs) were monitored throughout the duration of the study.

To monitor for possible AEs, vital signs, hematology and clinical chemistry laboratory parameters, ECG readings, neurological examination findings, and EEG parameters and abnormal findings were recorded at each visit.

### Statistical Analysis

Descriptive statistics were calculated for plasma and urine PK parameter and concentration data and summarized by study day and time point. Arithmetic means, coefficient of variation (CV), standard deviation, median, minimum, and maximum values, and number of observations were calculated for all PK parameters and trough concentration data. Except for Tmax, the geometric mean, geometric standard deviation, and geometric CV were provided for all PK parameter and concentration data.

### Cohorts 1-3 Results:

The following pharmacokinetic parameters were determined for each cohort: maximum plasma concentration (Cₘₐₓ, observed, Day 1 only); time to reach the maximum plasma concentration (Tₘₐₓ, observed, Day 1 only); and area under the plasma concentration-time curve from time 0 to 24 hours post dose (AUC₀₋ₜₐᵤ).

The following steady state pharmacokinetic (Day 14) parameters were determined: t_{1/2}: elimination half-life associated with the terminal slope (λz) of the semi-logarithmic drug concentration-time curve, calculated as 0.693/λz; C_{max,ss}: maximum plasma concentration (observed); T_{max,ss}: time to reach the maximum plasma concentration (observed); AUCₛₛ: area under the plasma concentration-time curve from 0 to 24 hours post dose; AUC_{inf}: area under the plasma concentration-time curve from 0 to infinite time; C_{avg}: average concentration over dosing interval; CLss/F: steady state clearance; and Vz/F: volume of distribution of terminal phase.

Table 1 shows a summary of the observed PK parameters on Day 1.

TABLE 1
Summary of Parameters for Compound 1 (calculated
from Day 1 data for Cohorts 1-3)

| | | | | | Ctrough |
|---|---|---|---|---|---|
| | | Tₘₐₓ | Cₘₐₓ | AUC₀₋ₜₐᵤ | (Day 2) |
| Treatment | Statistic | (h) | (ng/mL) | (h*ng/mL) | (ng/mL) |
| Cohort 1 | Mean | 1.056 | 57.367 | 378.685 | 4.207 |
| | SD | 0.273 | 13.169 | 119.963 | 2.298 |
| | Min | 0.75 | 39.10 | 219.21 | 1.69 |
| | Median | 1.00 | 55.00 | 394.97 | 3.89 |
| | Max | 1.50 | 76.00 | 581.62 | 8.01 |
| | % CV | 25.9 | 23.0 | 31.7 | 54.6 |
| | Geo Mean | 1.027 | 56.016 | 361.762 | 3.716 |
| | CV % g mean | 25.08 | 23.59 | 33.22 | 56.06 |
| Cohort 2 | Mean | 2.028 | 132.511 | 842.141 | 9.353 |
| | SD | 2.323 | 50.359 | 181.943 | 3.3335 |
| | Min | 0.75 | 34.60 | 449.41 | 5.06 |
| | Median | 1.00 | 156.00 | 899.25 | 8.62 |
| | Max | 8.00 | 195.00 | 1030.39 | 14.50 |
| | CV % | 114.6 | 38.0 | 21.6 | 35.7 |
| | Geometric Mean | 1.432 | 120.018 | 820.402 | 8.805 |
| | CV % Geometric | 91.88 | 57.47 | 26.22 | 38.93 |
| | Mean | | | | |
| Cohort 3 | Mean | 1.139 | 363.889 | 2343.762 | 19.024 |
| | SD | 0.435 | 96.801 | 585.039 | 10.024 |
| | Min | 0.75 | 213.00 | 1405.82 | 5.44 |
| | Median | 1.00 | 341.00 | 2356.80 | 20.40 |
| | Max | 2.00 | 511.00 | 3128.23 | 32.30 |
| | CV % | 38.2 | 26.6 | 25.0 | 52.7 |
| | Geometric Mean | 1.074 | 352.127 | 2274.106 | 16.049 |
| | CV % Geometric | 36.72 | 28.13 | 27.16 | 75.74 |
| | Mean | | | | |

Table 2 shows a summary of the observed PK parameters on Day 14 for Cohorts 1-3.

TABLE 2
Summary of Parameters for Compound 1 (calculated from Day 14 data)

| | | t_{1/2} | T_{max, ss} | C_{max, ss} | AUCinf | CLss/F | Vz/F | AUCss | C_{avg} |
|---|---|---|---|---|---|---|---|---|---|
| Treatment | Statistic | (h) | (h) | (ng/mL) | (h*ng/mL) | (L/h) | (L) | (h*ng/mL) | (ng/mL) |
| Cohort 1 | Mean | 14.772 | 1.139 | 66.333 | 623.3 | 31.415 | 655.194 | 506.288 | 21.093 |
| | SD | 2.260 | 0.435 | 13.733 | 182.7 | 8.313 | 130.993 | 129.280 | 5.387 |
| | Min | 11.48 | 0.75 | 41.50 | 351 | 19.57 | 464.09 | 306.85 | 12.79 |
| | Median | 15.26 | 1.00 | 66.00 | 595.2 | 30.41 | 656.82 | 493.56 | 20.57 |
| | Max | 17.53 | 2.00 | 91.80 | 1003 | 48.88 | 842.72 | 766.41 | 31.93 |
| | % CV | 15.3 | 38.2 | 20.7 | 29.3 | 26.5 | 20.0 | 25.5 | 25.5 |
| | Geo Mean | 14.613 | 1.074 | 65.010 | 600.228 | 30.497 | 642.968 | 491.871 | 20.497 |
| | CV % Geo Mean | 15.90 | 36.72 | 21.98 | 29.87 | 26.08 | 21.16 | 26.08 | 26.08 |
| Cohort 2 | Mean | 12.701 | 0.964 | 164.286 | 1226.440 | 30.949 | 570.566 | 1016.979 | 35.089 |
| | SD | 1.233 | 0.267 | 46.248 | 284.205 | 7.912 | 180.389 | 222.845 | 7.581 |
| | Min | 10.47 | 0.75 | 103.00 | 805.89 | 23.42 | 405.14 | 646.59 | 18.73 |
| | Median | 12.90 | 1.00 | 150.00 | 1268.36 | 30.12 | 513.72 | 995.94 | 37.47 |
| | Max | 14.19 | 1.50 | 222.00 | 1544.30 | 46.40 | 949.89 | 1280.73 | 42.93 |
| | CV % | 9.7 | 27.7 | 28.2 | 23.2 | 25.6 | 31.6 | 21.9 | 21.6 |
| | Geo Mean | 12.647 | 0.937 | 158.580 | 1196.442 | 30.178 | 550.639 | 994.095 | 34.183 |
| | CV % Geo Mean | 10.11 | 25.67 | 29.64 | 24.88 | 24.02 | 28.09 | 24.02 | 26.22 |
| Cohort 3 | Mean | 12.230 | 0.969 | 355.000 | 2373.932 | 32.142 | 574.599 | 2084.130 | 86.839 |
| | SD | 1.323 | 0.248 | 157.003 | 823.150 | 11.640 | 242.861 | 729.880 | 30.412 |
| | Min | 9.79 | 0.75 | 145.00 | 1368.69 | 17.36 | 302.08 | 1190.49 | 49.60 |
| | Median | 12.42 | 1.00 | 371.50 | 2439.64 | 28.45 | 531.34 | 2109.71 | 87.90 |
| | Max | 13.82 | 1.50 | 565.00 | 3931.46 | 50.40 | 971.28 | 3457.01 | 144.04 |
| | CV % | 10.8 | 25.6 | 44.2 | 34.7 | 36.2 | 42.3 | 35.0 | 35.0 |
| | Geo Mean | 12.164 | 0.944 | 320.410 | 2253.016 | 30.398 | 533.456 | 1973.814 | 82.242 |
| | CV % Geo Mean | 11.36 | 23.83 | 54.42 | 35.80 | 36.78 | 42.61 | 36.78 | 36.78 |

Conclusions: Compound 1 underwent rapid absorption with an approximate proportional increase in Cₘₐₓ and AUC parameters at steady state. Where steady state data was available, mean t_{1/2} was varied between 12.23 and 14.77 hours and steady state clearance was determined at approximately 31 L/hour.

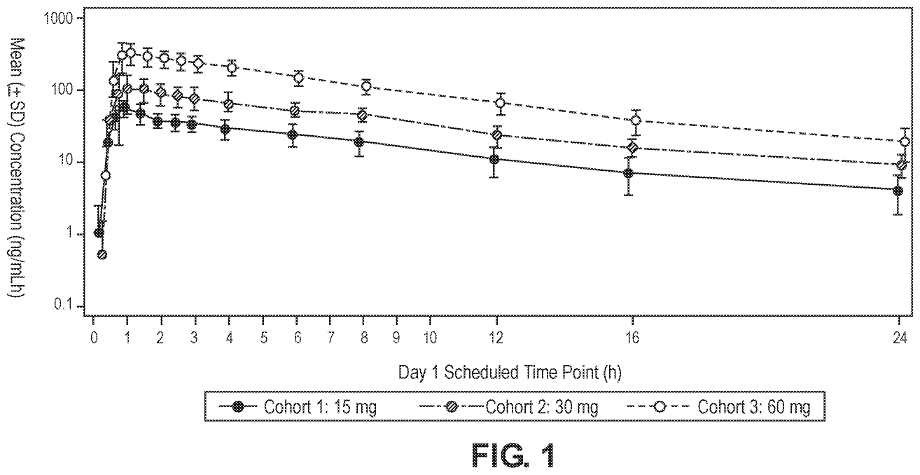

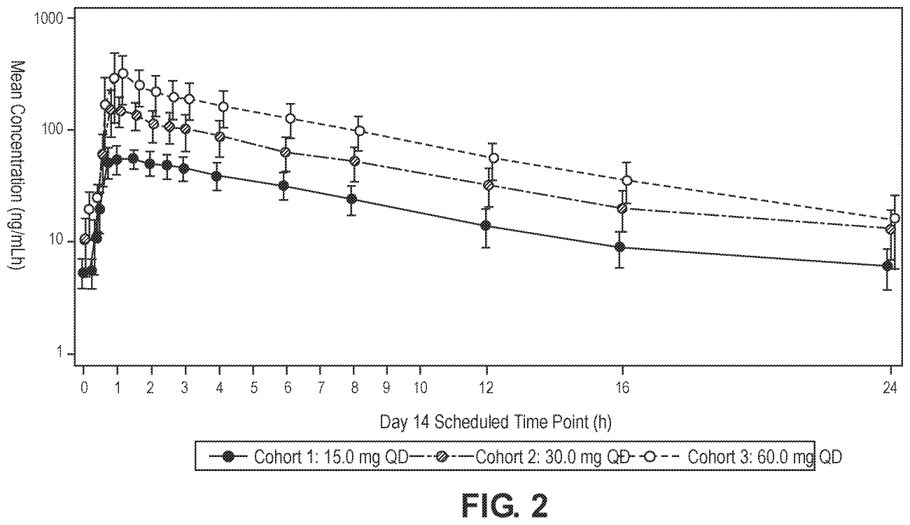

Rapid absorption was observed with a Tmax occurring within the first 2 hours of dosing (Tables 1-2). An accumulation factor of the dosing interval (ratio of AUC_{Day14}/AUC_{Day1}) was approximately 1.25 for Cohort 1 and Cohort 2 (Tables 1 and 2). For Cohort 3, the accumulation factor was 0.89 (Tables 1 and 2). The mean plasma concentration (ng/mL) for the first 24 hours after the initial dose is illustrated in FIG. 1. The mean plasma concentration (ng/mL) for the 24 hours after the last dose is illustrated in FIG. 2.

Mean t_{1/2} for Cohort 1, Cohort 2 and Cohort 3 was 14.77±2.26 hours, 12.70±1.23 hours and 12.23±1.32 hours, respectively. Steady state clearance was approximately 31 L/hour for the three cohorts at Day 14, and the mean Vz/F was 655, 570 and 574 L for Cohorts 1, 2 and 3, respectively. A proportional increase in Cavg, AUCss and AUCinf was observed between Cohorts 1 and 3 (Tables 1 and 2).

In Cohorts 1-3, there were no Serious Adverse Events and no clinically relevant vital sign, ECG, or lab abnormalities. Additionally, several subjects experienced elevated mood at doses of 30 mg and 60 mg.

### Cohort 4 Results:

Table 3 shows a summary of the observed PK parameters on Days 1 (fasted) and Day 5 (fed).

TABLE 3
Compound 1 Mean (SD) PK Parameters Following Oral
Administration for Food Effect Cohort Parameter

| | Fasted (Day 1) n = 10 | | Fed (Day 1) n = 10 | |
|---|---|---|---|---|
| Cₘₐₓ (ng/mL) | 84.18 | (48.245) | 45.36 | (10.74) |
| Tₘₐₓ (hours) | 1.00 | (0.75, 1.77) | 3.25 | (1.00, 6.00) |
| AUC₀₋ₗₐₛₜ (h · ng/mL) | 597.7 | (302.85) | 629.9 | (183.63) |
| AUC_{inf} (h · ng/mL) | 602.3 | (304.84) | 634.2 | (185.13) |
| t_{1/2} (hours) | 11.22 | (1.7749) | 11.26 | (0.86406) |
| AUC %_{Extrap} | 0.8702 | (0.50874) | 0.6632 | (0.28199) |

For Tmax, Median (Minimum, Maximum) values are displayed.

Table 4 shows a statistical analysis of the effect of a high-fat meal on Cₘₐₓ and AUC₋ₜ for the 30 mg dose.

TABLE 4
Compound 1 Cₘₐₓ and AUC₀₋ₜ

| | Fed/Fasted Ratio | 90% Confidence Interval |
|---|---|---|
| Cₘₐₓ (ng/mL) | 0.64 | (0.46, 0.88) |
| AUC₀₋ₜ (h · ng/mL) | 1.17 | 629.9 (183.63) |

### Conclusions

Subjects displaying high concentrations following fasted conditions also tended to show higher concentrations (relative to others within the treatment group) following fed conditions. Similarly, subjects with low Compound 1 concentrations following fasted conditions tended to have low Compound 1 concentrations following fed conditions. The t_{1/2} was similar for both fasted and fed conditions (Table 3).

In the 30 mg dose group, overall drug exposure as measured by area under the concentration curve, or AUC, of Compound 1 increased by only 1.17-fold in the fed state versus in the fasted state (Table 4). The primary effect of food was observed in the maximum drug exposure, or Cmax, which was decreased by 0.64-fold under fed conditions. These data establish an absence of food effect on bioavailability as defined by FDA guidelines. These findings indicate that Compound 1 does not need to be taken with food to achieve therapeutic exposures.

### Example 2

Patients with severe major depressive disorder (MDD) aged 18 to 65 years were treated with an oral suspension of Compound 1 to study the safety, tolerability, pharmacokinetics, and efficacy of Compound 1 in the treatment of severe MDD.

The study showed that patients receiving a daily dose of 45 mg or 80 mg of Compound 1 exhibited reduced symptoms of moderate to severe MDD.

### Study Design

The study is an open-label, study comprised of two dosing periods, Part A and Part B, during which patients were treated with an oral suspension of Compound 1. Part A was an open-label assessment of two dose levels of Compound 1 (45 mg qHS Cohort 1 and 80 mg qHS Cohort, i.e., once daily administration at bedtime) administered for 7 days inpatient followed by 7 days outpatient. Compound 1 was administered at 4 p.m. on Day 1 in order to collect post-dosing PK samples that could not be collected with qHS dosing.

Part B is an assessment of a single level of Compound 1 (40 mg qHS) administered for 28 days in an outpatient setting.

Each part of the clinical trial will enroll an independent set of participants. Participants from Part A will not be eligible to enroll in Part B. Each participant will complete three periods: Screening, Treatment Period (14 days of dosing PO daily for Part A, 28 days of dosing PO daily for Part B), and Safety Follow-up.

Screening Period: The screening period for Part A was up to 14 days in duration (Day −14 to Day −1), and the screening period for Part B was up to 28 days in duration (Day −28 to Day −1). Prior to any clinical trial procedures, participants will provide written informed consent to participate in the clinical trial. Screening assessments will include: medical history, demographics, vital signs, physical examination (including height and weight), drug screen, clinical laboratory tests, an electrocardiogram (ECG), the Mini International Neuropsychiatric Interview (MINI), the Antidepressant Treatment History Questionnaire (ATRQ), HAM-D, and C-SSRS assessment.

Treatment Period: The treatment period for Part A was 14 days in duration, and the treatment period for Part B was 28 days in duration.

In Part A, participants were administered daily doses of Compound 1 (45 mg Cohort and 80 mg Cohort) at 4 p.m. on Day 1 and qHS on all other study days for 7 days inpatient followed by 7 days outpatient (with subsequent Cohorts receiving up to 120 mg qHS). Participants checked into the clinic on Day 1. During this period the following assessments were performed: adverse event assessments, vital signs, physical examinations, blood and urine samples for safety assessments, ECGs, C-SSRS assessments, blood samples for PK, and efficacy assessments (HAM-D, MADRS, HAM-A, CGI [CGI-S and CGI-I], PSQI, and SDQ). Baseline assessments occurred on Day 1 prior to administration of study drug. On Day 8, prior to discharge, participants were supplied with sufficient Compound 1 to complete 7 days of dosing.

In Part B, participants were administered daily doses Compound 1 (40 mg qPM Cohort) or a placebo for 28 days as outpatients, randomized in a 1:1 ratio to received double-blind treatment. Study visits will be conducted on Days 1, 8, 15, 22, and 29, with Day 4 clinical assessments conducted via telehealth procedures. During this period the following assessments will be performed: adverse event assessments, vital signs, physical examinations, blood and urine samples for safety assessments, ECGs, C-SSRS assessments, blood samples for PK, and efficacy assessments (HAM-D, MADRS, HAM-A, CGI [CGI-S and CGI-I], PSQI, and SDQ). Baseline assessments occurred on Day 1 prior to administration of study drug.

### Safety Follow-Up Period

In Part A, participants returned to the clinic for safety follow-up visits on Day 15, Day 21 (±1 day), and Day 28 (±1 day). For Part B, participants returned to the clinic for safety follow-up visits on Day 36 and Day 43. During these visits the following assessments will be performed: adverse event assessment, vital signs, physical examination, drug screen (Day 15 only), clinical laboratory tests, an ECG, C-SSRS assessment, and efficacy assessments (HAM-D, MADRS, HAM-A, CGI [CGI-S and CGI-I], SDQ, and PSQI).

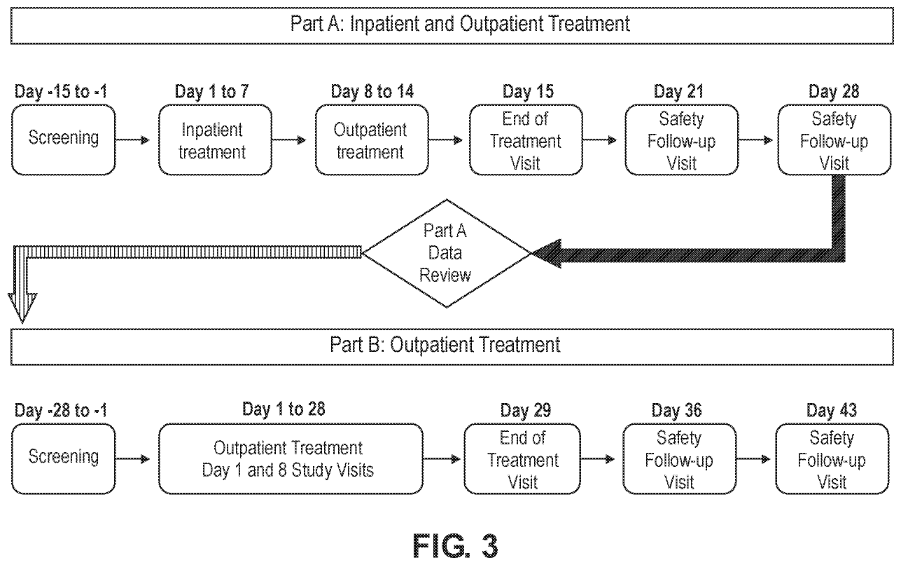

A diagram of the Part A and Part B study protocol is provided in FIG. 3.

### Patient Population:

This trial will enroll participants with severe MDD without confounding medical or psychiatric disorders that would jeopardize the safety or scientific validity of the clinical trial. Eligibility will be confirmed by Sponsor or designee.

Number of Participants: The clinical trial is planned to include between 24 and up to a total of 60 participants across both parts (Part A and B). In Part A, 13 participants were treated in the 45 mg Cohort, and 7 participants were treated in the 80 mg Cohort. In Part B, between 10 to 12 participants are planned to be treated in the 40 mg Cohort.

Inclusion Criteria: A participant must meet the following criteria at Screening to be eligible to participate in this clinical trial:
Males and females between the ages of 18 and 65 years (inclusive).
Weight of at least 50 kg with body mass index between 18 and 30 kg/m2 (inclusive).
For Part A, have a clinical diagnosis of severe MDD that has been present for at least a 4-week period, with a HAM-D score at screening of ≥22. For Part B, have a DSM-5 diagnosis of recurrent MDD as defined by the MINI, are currently experiencing a current major depressive episode of at least 8 weeks and not more than 24 months in duration, and have a HAM-D17 total score ≥23 and a CGI-S score of ≥4 at both screening and on Day 1.
All chronic medications or interventions, especially those for depression, must have been stable for at least four weeks prior to Screening and remain stable throughout the clinical trial.
Use of a medically acceptable method of contraception throughout the duration of the clinical trial and for three months thereafter.
Willing to sign an informed consent document indicating that he/she understands the purpose of the clinical trial and the procedures that are required for the clinical trial and that he/she is willing to participate in the trial and complete all applicable assessments and comply with the protocol.

Exclusion Criteria: A participant who meets any of the following criteria at Screening (unless otherwise specified) will be excluded from this clinical trial:
Ongoing or history of any medical or surgical condition that, in the judgment of the Investigator, might jeopardize the participant's safety or interfere with the absorption, distribution, metabolism or excretion of Compound 1.
Known hypersensitivity to any component of the formulation of Compound 1.
Taking any of the following medicines: Bupropion, Buspirone, Midazolam, Alprazolam, Nefazodone, Trazadone, Carbamazepine, Clonazepam, Quazepam, Tiagabine, Aripiprazole, olanzapine, quetiapine, brexpiprazole, and Triazolam.
Use of any experimental or investigational drug or device within 30 days prior to first dose of Compound 1 or 5 half-lives of Compound 1, whichever is longer.
Clinically significant unstable medical or psychiatric condition, other than severe MDD and moderate to severe anxiety in the opinion of the Investigator.
Clinically significant laboratory abnormalities that in the opinion of the Investigator would jeopardize the safe conduct of the trial.
Clinically significant abnormalities in a 12-lead ECG at Screening, per the review of a cardiologist.
History of treatment resistant depression in the current episode; defined as having failed three adequate trials of antidepressant therapy with treatments from at least two different therapeutic classes for a sufficient amount of time over which a beneficial effect is generally expected as determined with the ATRQ.
History of a suicide attempt in the last 2 years.
History of bipolar disorder.
History of a psychotic episode in last 2 years, or any diagnosis of psychotic disorder such as schizophrenia, schizoaffective disorder.
History of alcohol abuse within the past two years or daily consumption of more than 4 standard alcohol-containing beverages for males or more than 2 standard alcohol-containing beverages for females.
Has ingested alcohol in the 24-hour period prior to Day 1 of the clinical trial or is unwilling to abstain from drinking alcohol throughout the treatment period.
History of substance abuse in the past 2 years or a positive drug screen. A positive cannabis screen can be repeated.
History of seizures in the past 5 years or being treated for seizures in the last 5 years.
Psychosocial or addictive disorders that would interfere with participant's ability to give informed consent or could compromise compliance with the protocol.
Any other significant disease, disorder or abnormalities, that, in the opinion of the Investigator, may either put the participant at risk because of participation in the clinical trial, may compound the result of the clinical trial, or affect the participant's ability to participate in the clinical trial.

### Dosing:

Part A: Patients in Part A were treated with Compound 1 for 14 consecutive days, unless dosing is halted by the Safety Review Committee (SRC). Patients were administered daily doses of Compound 1 for 7 days inpatient followed by 7 days outpatient.

In the 45 mg Cohort, patients were administered once daily dose of 45 mg of Compound 1 at bedtime for 7 days inpatient followed by once daily dose of 45 mg of Compound 1 at bedtime for 7 days outpatient.

In the 80 mg Cohort, patients were administered once daily dose of 80 mg of Compound 1 at bedtime for 7 days inpatient followed by once daily dose of 80 mg of Compound 1 at bedtime for 7 days outpatient.

Part B: Patients in Part B were treated with 40 mg (two 20 mg tablets) of Compound 1 for 28 consecutive days, unless dosing was halted by the Safety Review Committee (SRC). Patients were administered daily doses of Compound 1 for 14 days as outpatients.

Blood and urine will be obtained during each treatment period at designated times for PK and other analyses (see below). Standard safety assessments will also be measured during each treatment period.

### Formulation

The Compound 1 drug product will be formulated as a suspension having a composition as summarized in Table 5. The Compound 1 oral suspension is planned to contain from 1 to 20 mg/mL of Compound 1.

TABLE 5
Compound 1 Composition of Drug Product
Suspension, 1 mg/mL to 20 mg/mL

| | | Amount, |
|---|---|---|
| Ingredient | Purpose | mg/mL |
| Compound 1 | Active | 1-20 mg/mL |
| Hypromellose 2910, 4000cP, | Suspension | 5.0 |
| USP | stabilizer | |
| Poloxamer 188, USP | Dispersing agent | 5.0 |
| Purified Water, USP or higher | Excipient | q.s. |
| quality | | 1.0 mL |
| To make | | |

A placebo to match the Compound 1 oral suspension will be manufactured having substantially the same composition as the active drug product but without Compound 1 (active pharmaceutical ingredient). However, microcrystalline cellulose will be used to mimic the appearance of the suspended Compound 1. The composition of the Placebo is summarized in Table 6.

TABLE 6
Composition of Placebo Drug Product Suspension

| | | Amount, |
|---|---|---|
| Ingredient | Purpose | mg/mL |
| Microcrystalline Cellulose, NF | API simulant | 5.0-20.0* |
| (MCC) | | |
| Hypromellose 2910, 4000cP, | Suspension | 5.0 |
| USP | stabilizer | |
| Poloxamer 188, USP | Dispersing agent | 5.0 |
| Purified water, USP or higher | Excipient | q.s. |
| quality | | 1.0 mL |
| To make | | |

### Pharmacokinetic (PK) Assessments

PK parameters (e.g., Cₘₐₓ, Tₘₐₓ, occurrence of steady state, etc.) for MDD patients in each cohort will be compared to assess the suitability of Compound 1 suspension for the treatment of MDD. Data will be obtained from the blood plasma samples collected from each cohort according to the schedule provided.

Plasma samples will be analyzed to determine Compound 1 concentrations using a validated assay method. Pharmacokinetic variables (including but not limited to Cₘₐₓ, and Tₘₐₓ) will be calculated using non-compartmental analysis. PK parameters for Compound 1 will be derived from the plasma concentration data using non-compartmental analysis with Phoenix™ WinNonlin® v 8.0 (Pharsight Corporation, USA).

### Protocol:

Blood: Part A: For Part A, blood samples were collected on Days 1, 2, 3, 4, 5, 6, 7, 15 and 28 at the following time points: Days 1-7 at about 1 hour pre-dose; Days 1-7 at about 1 hour after dosing, Day 15 and Day 28. Part B: For Part B, blood samples will be collected on Days described for Part A.

The occurrence of steady state will be assessed by visual inspection of individual participant trough concentration time course. Accumulation ratio will be estimated by comparing the Day 7 and Day 1 trough plasma concentrations of Compound 1.

### Pharmacodynamic Assessment

During Parts A and B, the following clinical scores will be assessed to determine the depression symptoms experienced by subjects: total HAM-D value, MADRS, HAM-A, CGI and, in particular, the CGI-S and CGI-I subscales, PSQI, and SDQ.

Part A: In Part A, HAM-D, MADRS, HAM-A, CGI-S, and CGI-I values were collected in the morning of Days 1, 2, 3, 4, 5, 6, 7, 8, 15, 21 and 28. PSQI and SDQ values will be collected on Days 1, 8, 15, 21 and 28. Part B: In Part B, HAM-D, MADRS, HAM-A, CGI-S, CGI-I, PSQI and SDQ values will be collected on the days described for Part A.

HAM-D Response was defined as a reduction from baseline of ≥50% in total HAM-D score. HAM-D remission was defined as a total HAM-D score of ≤7.

### Safety Assessments/Monitoring

To monitor for possible adverse events, vital signs, hematology and clinical chemistry laboratory parameters, ECG readings, neurological examination findings, and EEG parameters and abnormal findings were recorded at each visit.

### Statistical Analysis

Efficacy analysis: For both parts of the clinical trial, the HAM-D total score and change from baseline values will be summarized by treatment group and time point. In addition, the change from baseline in HAM-D total score will also be analyzed using paired t-tests or similar methods. The null hypothesis of this test is that the mean difference in HAM-D total score between paired observations (i.e., pre- and post-treatment) is zero. Similar analysis methods will be used for all secondary and exploratory efficacy variables.

PK analysis: Plasma concentrations of Compound 1 will be summarized using descriptive statistics by time point. The occurrence of steady state will be assessed by visual inspection of individual participant trough concentration time course. Accumulation ratio will be estimated by comparing the Day 7 and Day 1 trough plasma concentrations of Compound 1.

A validated bioanalytical method will be utilized for the determination of plasma concentrations of Compound 1. Plasma samples may also be used for additional exploratory bioanalytical method development and/or metabolite characterization purposes only.

Plasma concentrations will be summarized using descriptive statistics. If the concentration of Compound 1 is reported as below the limit of quantitation (BLQ), a value of zero will be assigned for the purposes of calculating descriptive statistics. Individual and mean concentrations will be presented as BLQ if below the bioanalytical quantitation limit.

The PK population is defined as all participants with at least one valid bioanalytical plasma concentration of Compound 1.

### Part A Efficacy Results:

Efficacy (as determined by reduction Mean Change from Baseline in HAM-D score) was observed in patients that were undergoing their first course of antidepressant treatment, as well as in patients that failed previous courses of antidepressant treatment. Efficacy was observed in the absence of a background antidepressant (i.e., Compound 1 administered as monotherapy) and in presence of an antidepressant (i.e., Compound 1 administered in combination with another antidepressant).

45 mg Cohort: On Day 7, 11 of the 13 patients met the HAM-D Response or Remission criterion. On Days 7 and 15, the least squares Mean Change from Baseline in HAM-D score was −17.8 and −13.2, respectively. On Day 15, 8 of the 13 patients met the HAM-D Response or Remission criteria.

80 mg Cohort: On Day 7, all 7 patients met the HAM-D Response or Remission criterion. On Days 7 and 15, the least squares Mean Change from Baseline in HAM-D score was −20.0 and −16.0, respectively. On Day 15, 6 of the 7 patients met the HAM-D Response or Remission criteria.

The 80 mg dose was well tolerated with no change in Adverse Event profile compared to the 45 mg Cohort.

### Part A PK Results:

Using statistical modelling based upon the PK studies conducted in Example 1, the following PK parameters that correlate to the reduction in HAM-D score observed in the 45 mg and 80 mg Cohorts were predicted in Table 7:

TABLE 7
Predicted PK parameters

| Cohort | Model | Cₘₐₓ (ng/mL | AUC₀₋₂₄ (h · ng/mL) |
|---|---|---|---|
| 45 mg | Model 1 | 47.2-175 | 341-1420 |
| | Model 2 | 32-129 | 337-1410 |
| 80 mg | Model 1 | 83.9-312 | 606-2530 |
| | Model 2 | 56.9-229 | 599-2500 |
| | Model 3 | 150-556 | 1080-4510 |

### Example 3

Patients with severe major depressive disorder (MDD) aged 18 to 65 years were treated with an oral suspension or tablet of Compound 1 to study the effect of food and time of administration on the pharmacokinetics, as well as the safety and tolerability of these formulations.

### Design:

The clinical trial is composed of the following four parts:

Part A is an open-label, non-randomized, fixed-sequence crossover design to evaluate Compound 1 at 2 different administration times (4 p.m., approximately 4 hours after lunch and 2 hours prior to dinner, and at 8 p.m., approximately 4 hours after dinner). 16 participants were administered an oral suspension of Compound 1.

Part B is an open-label, randomized, crossover design to examine the effect of a high-fat, high-calorie meal on Compound 1 exposure following administration of the tablet comprising Compound 1 in up to 16 participants.

Part C is a randomized, participant- and Investigator-blinded, Sponsor-unblinded, placebo-controlled, parallel-group design to evaluate the pharmacokinetics and safety of single ascending doses of a Compound 1 citrate tablet. One cohort of 16 participants was planned, with 12 participants receiving Compound 1 and 4 participants receiving placebo. Part C also evaluated administration of a single dose level at 8 p.m., both 2 hours and 4 hours after dinner.

Part D is a randomized, participant- and Investigator-blinded, Sponsor-unblinded, placebo-controlled, parallel-group design to evaluate the pharmacokinetics and safety of multiple ascending doses of the Compound 1 citrate tablet. Three cohorts of 12 participants were planned, with 9 participants receiving Compound 1 and 3 participants receiving placebo. The exposure to 3 major human metabolites will also be quantified using validated methods.

### Results:

Preliminary pharmacokinetics data from Part A, where 45 mg and 60 mg of oral suspension were administered (Cohort 1), and Part C, where 40 mg (2×20 mg), 60 mg (3×20 mg) and 80 mg (4×20 mg) of the tablet formulation were administered, have been analyzed and are summarized below.

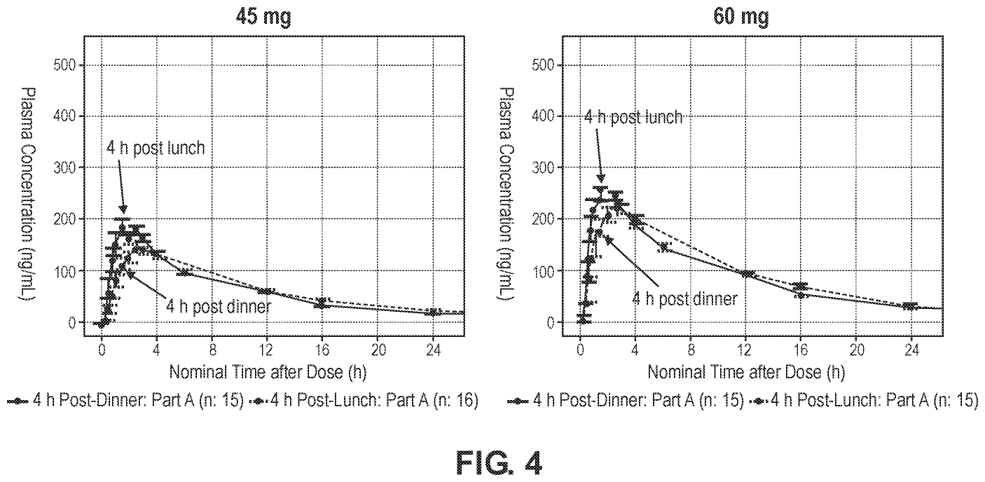

Administration of a suspension comprising Compound 1 at either 4 p.m. or 8 p.m. resulted in appearance of Compound 1 concentrations at the first post-dose timepoint assessed (0.25 hours) that remained detectable through to the last evaluated timepoint (72 hours). For both doses administered (45 mg and 60 mg), mean Compound 1 concentrations were higher within the first 2 to 3 hours following administration at 4 p.m. as compared to 8 p.m. (FIG. 4). Further, tₘₐₓ was delayed and Cₘₐₓ was reduced when Compound 1 was administered at 8 p.m. as compared to 4 p.m. (Table 8). Despite the reduction in Cₘₐₓ, AUC_{inf} was comparable between the 2 administration times for both dose levels tested. Mean t_{1/2} ranged from 11.1 hours to 12.6 hours.

TABLE 8
Summary of Plasma Pharmacokinetic Parameters after administration
of an oral suspension of Compound 1 at 4 p.m. and 8 p.m.

| | 45 mg 4 PM^{a} | | 45 mg 8 PM^{b} | | 60 mg 4 PM^{a} | | 60 mg 8 PM^{b} | |
|---|---|---|---|---|---|---|---|---|
| | (N = 16) | | (N = 15) | | (N = 15) | | (N = 15) | |
| Metric (Units) | Mean (SD) | | Mean (SD) | | Mean (SD) | | Mean (SD) | |
| AUC_{inf} (h · ng/mL) | 1800 | (530) | 1880 | (412) | 2800 | (601) | 2930 | (516) |
| Cₘₐₓ (ng/mL) | 207 | (47.9) | 157 | (32.5) | 282 | (39.0) | 249 | (42.2) |
| tₘₐₓ (h)^{c} | 1.5 | (0.75, 3.0) | 2.5 | (1.0, 4.0) | 1.5 | (0.75, 3.0) | 2.5 | (0.5, 4.0) |
| t_{1/2} (h) | 12.6 | (1.92) | 11.3 | (1.36) | 12.0 | (1.31) | 11.1 | (1.48) |

AUC_{inf} = area under the concentration-time curve from zero to infinity; AUCₜ = area under the concentration-time curve from time 0 to time t; Cₘₐₓ = maximum observed plasma concentration; h = hour; N = number of participants at each dose level; SD = standard deviation; t_{1/2} = terminal half-life; tₘₐₓ = time to maximum plasma concentration.
^{a}Dose administered 4 hours after lunch and 2 hours before dinner.
^{b}Dose administered 4 hours after dinner.
^{c}Median (minimum, maximum) reported for tₘₐₓ.

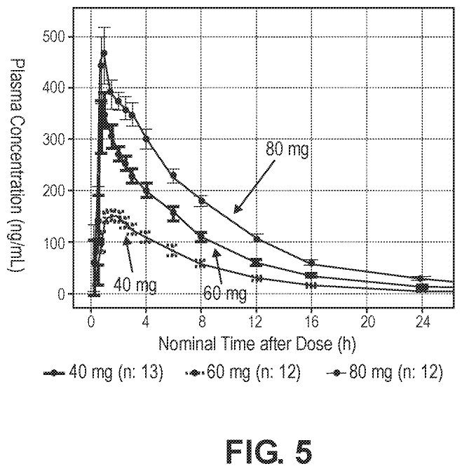

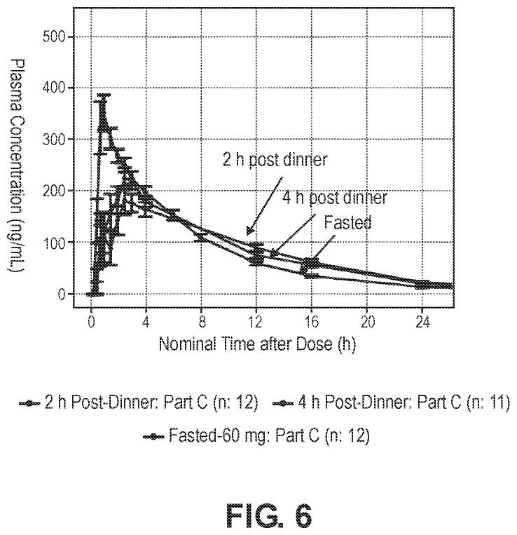

A tablet comprising Compound 1 was administered as single ascending doses of 40 mg, 60 mg and 80 mg under fasted conditions in the morning (FIG. 5). The 60 mg dosage was administered on two additional occasions at 8 p.m., once 4 hours after dinner and once 2 hours after dinner (FIG. 6). Administration of the Compound 1 citrate tablet under fasted conditions resulted in a rapid rise in concentrations with a median tₘₐₓ of 1.0 to 1.5 hours. A greater than dose-proportional increase in Compound 1 exposure was observed under fasted conditions with an approximate 3-fold increase in exposure over a 2-fold increase in dose (from 40 mg to 80 mg). However, the exposure was closer to dose-proportional between 60 mg and 80 mg, suggesting that the non-proportionality observed over the entire dose range occurred primarily as a result of the 40 mg dose.

Similar to the oral suspension, when the Compound 1 citrate tablet was administered at 8 p.m., regardless of the duration after dinner, tₘₐₓ was delayed to 2.5 hours, and Cₘₐₓ was reduced (about 40%) as compared to fasted conditions. However, AUC_{inf} was comparable across the 3 administration conditions tested. Mean t_{1/2} ranged from 9.6 to 11.9 hours and appeared to be independent of dose or administration time.

TABLE 9
Summary of Plasma Pharmacokinetic Parameters after administration
of an oral suspension of Compound 1 at 9 a.m. and 8 p.m.

| | 9 a.m. administration | | | 8 p.m. administration (60 mg) | |
|---|---|---|---|---|---|
| | 40 mg | 60 mg | 80 mg | 2 h after dinner | 4 h after dinner |
| | (N = 13) | (N = 12) | (N = 12) | (N= 12) | (N = 11) |
| Metric (Units) | Mean (SD) | Mean (SD) | Mean (SD) | Mean (SD) | Mean (SD) |
| AUC_{inf} | 1390 (347) | 2610 (488) | 3980 (654) | 2510 (465) | 2550 (541) |
| (h · ng/mL) | | | | | |
| Cₘₐₓ (ng/mL) | 181 (32.1) | 408 (120) | 546 (126) | 222 (67.8) | 261 (81.0) |
| tₘₐₓ (h)^{a} | 1.5 (0.75, 2.5) | 1.0 (0.75, 1.5) | 1.0 (0.75, 2.5) | 2.5 (1.0, 12.0) | 2.5 (1.0, 3.0) |
| t_{1/2} (h) | 11.9 (1.0) | 11.5 (1.9) | 11.4 (0.88) | 9.6 (1.7) | 11.4 (1.2) |

AUC_{inf} = area under the concentration-time curve from zero to infinity; AUCₜ = area under the concentration-time curve from time 0 to time t; Cₘₐₓ = maximum observed plasma concentration; h = hour; N = number of participants at each dose level; SD = standard deviation; t_{1/2} = terminal half-life; tₘₐₓ = time to maximum plasma concentration.
^{a}Median (minimum, maximum) reported for tₘₐₓ.

As demonstrated by the data above, evening dosing of Compound 1 resulted in reduced Cₘₐₓ and increased tₘₐₓ compared to morning dosing of Compound 1. Surprisingly, the total drug exposure remained unchanged.

### Example 4

The effect of Compound 1 on the quantitative EEG (qEEG) was measured in a Phase 1 multiple ascending dose (MAD) trial to understand the pharmacodynamic effect of Compound 1 on GABA_{A} receptor activation. The frequency and amplitude of the detected electrical signals in response to treatment with a brain-active compound provide insights into brain function and brain state (e.g., awake, deep sleep, etc.) or pharmacological response. For instance, changes in power in the beta-band frequency can be used as a pharmacodynamic biomarker of GABA_{A} receptor activation in response to a brain active compound.

### Protocol:

EEG was recorded for 5 minutes in quiet eyes open condition using a standard 10-20 EEG electrode placement system at pre-dose and 1, 2, 4, 8 and 24 hours after dosing. Each lead was referenced to the Oz electrode. Artifacts were removed via visual inspections. EEG spectra were computed using Fourier transformation. The alpha-band frequency was set to 8.5-12.5 Hz, the beta-band frequency was set to 12.5 to 30 Hz. The ratio between each post-dose value and the associated pre-dose value for each electrode was used to calculate the fold EEG change in each frequency band so that a ratio of 1 represents no change from baseline. The mean of the central 9 electrodes was used for each subject and timepoint for final analysis.

### Results:

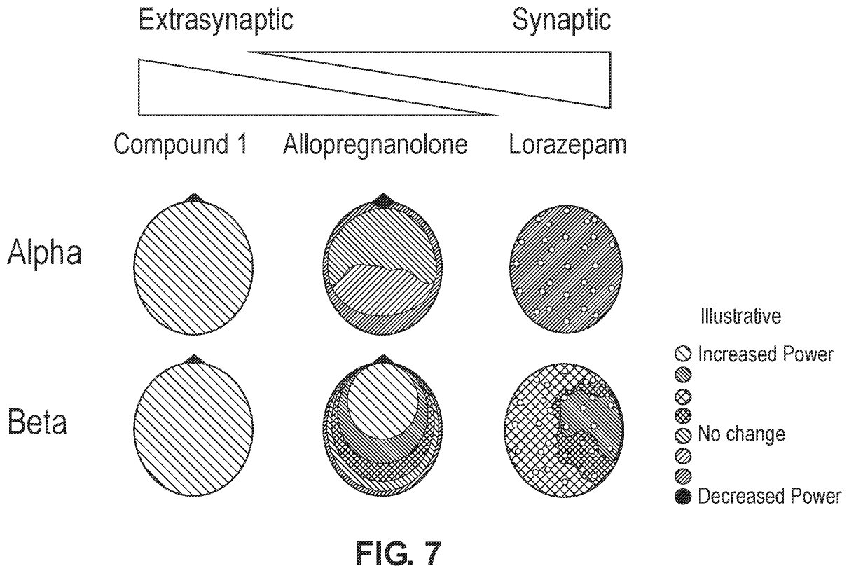

Changes in qEEG power were measured in volunteers to assess the effect of Compound 1 on GABA_{A} receptors in the brain on days 1 and 14 of this trial. Compound 1 produced marked increases in the power of the alpha-band and beta-band frequencies. Increases in the beta-band frequency are correlated with GABAergic activation, as previously shown by the marketed GABA_{A} PAMs, allopregnanolone and lorazepam. Compound 1 distinctly increases the alpha-band frequency, unlike allopregnanolone and lorazepam, which have shown to decrease the power of the alpha-band frequency (FIG. 7). It is believed this qEEG profile of Compound 1 is consistent with its extrasynaptic GABA_{A} receptor preference and differentiated pharmacological profile relative to benzodiazepines and other GABA_{A} PAMs in this class or NAS class.

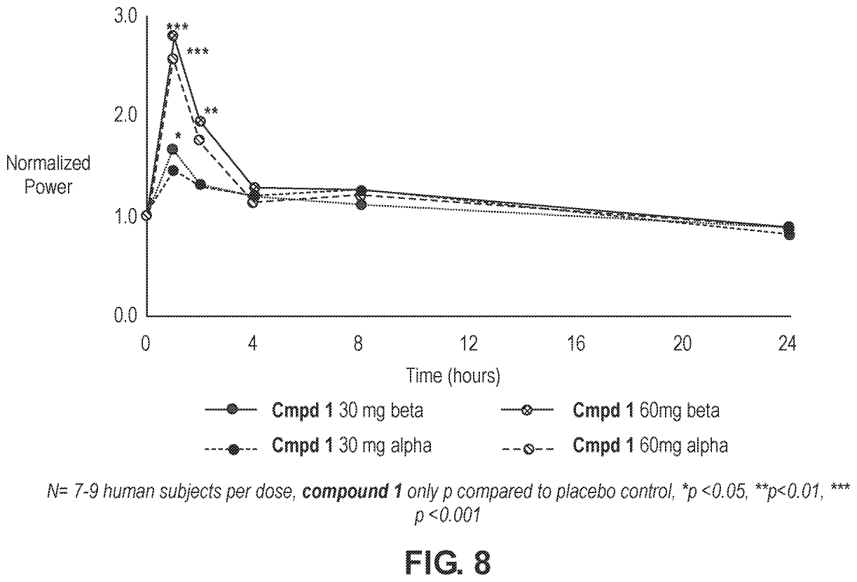

Moreover, increases in alpha-band and beta-band frequencies in the qEEG were strongly correlated with dose and Compound 1 levels in the blood. In these healthy volunteers at one hour post-dose on Day 1, Compound 1 dosed at 30 mg resulted in an average increase in qEEG alpha and beta power of approximately 1.5-fold and 1.6-fold compared to baseline. Compound 1 dosed at 60 mg resulted in an increase in this measure of 2.6-fold and 2.8-fold compared to baseline, respectively (FIG. 8). The effects on the qEEG alpha and beta power were sustained at Day 14. These data show that Compound 1 engaged GABA_{A} receptors in the brain and produced consistent effects on qEEG within the first hour after dosing with similar effects on Days 1 and 14. This finding also supports comparison and translation of the pharmacologic activity and qEEG data from the pre-clinical studies, where a 1.6-fold increase in beta power was associated with robust activity in animal models of anxiety and depression and was used to inform dose selection in subsequent clinical trials.

Notably, in the MAD study, Compound 1 showed increases in beta power up to 2.8-fold without achieving a maximum tolerated dose (MTD) or demonstrating any serious adverse events (SAEs) after a dose of 60 mg, and a higher dose of 80 mg dosed as a single dose in MDD patients for 14 days was also well-tolerated in a subsequent clinical trial. In a separate study, another molecule in development in the class resulted in degrees of sedation that were not tolerated at doses that resulted in increases in beta power by approximately 1.7-fold compared to baseline. This highlights the extrasynaptic GABA_{A} receptor preference and unique pharmacological profile of Compound 1 and its ability to achieve high levels of GABAergic activation with improved tolerability.

### Conclusions

From this study, it has been determined that Compound 1 is approximately 10-fold more selective PAM of the extrasynaptic form of GABA_{A} receptors compared to the synaptic form. In healthy volunteers, Compound 1 is observed to markedly increase qEEG power in the alpha-band and beta-band frequencies—unlike GABA_{A} receptor PAMs that only modulate synaptic GABA_{A} receptors, such as benzodiazepines, or that are equipotent at synaptic and extrasynaptic receptors, such as allopregnanolone, which decrease power in the alpha-band frequency. Without being bound by any particular theory, these data suggest that Compound 1 has a differentiated pharmacological profile to other GABA_{A} PAMs at therapeutic doses due to the relatively selective activation of extrasynaptic GABA_{A} receptors. By preferentially modulating extrasynaptic GABA_{A} receptors, it appears Compound 1 is able to uniquely activate the GABAergic target and can mediate antidepressant and anxiolytic activity without the significant sedation observed with less selective neuroactive steroids.

### Example 5

A rat model of tremor (Rat Harmaline Induced Tremor; rHIT) was used to assess the impact of Compound 1 on reducing the symptoms of tremor. Male SD rats (SLAC, Shanghai, China) having a body weight of about 250 g at the time of testing were fed ad libitum and kept in a 12:12 light-dark cycle room, with the lights on from 5 a.m. to 5 p.m, throughout the study. Rats were allowed to acclimate to standard laboratory conditions for at least 5-7 days prior to the experiment.

One day prior to testing, a naive rat was placed in the testing chamber, which was a plexiglass chamber with a piezoelectric plate attached at the bottom to transduce tremor behavior into an electrical signal. The rat was placed in the chamber in order to adjust the spontaneous activity baseline signal. The baseline was adjusted to be within the range of 3000-10000 V, which sensitivity level generally allows all the tremor signal to be detectable within the system.

On the day of testing, each rat was allowed to acclimate for at least thirty minutes in the test room. Prior to testing, rats were placed into the testing chamber for a five-minute offset to ensure the baseline activity was within the accepted range (3000-10000 V). At thirty minutes prior to harmaline administration (T=−30 minutes), rats were dosed intraperitoneally with either (1) 5 mL/kg of a vehicle control containing 15% Captisol; (2) 0.3 mg/kg (5 mL/kg) Compound 1; (3) 1 mg/kg (5 mL/kg) Compound 1; (4) 3 mg/kg (5 mL/kg) Compound 1; or (5) 10 mg/kg (5 mL/kg) Compound 1. Twenty minutes prior to harmaline administration (T=−20 minutes), an additional group of rats was dosed orally with 10 mg/kg propranolol. For each of the aforementioned six groups, n=15. Rats were then kept in the testing chamber to allow for a 20-minute baseline recording (T=−20 to 0 minutes) before harmaline treatment.

Next, at T=0 minutes, all groups were intraperitoneally administered 30 mg/kg (5 mL/kg) of harmaline formulated in a saline vehicle. The post-harmaline effect was measured for twenty minutes (T=10 to 30 minutes). The electrical signal of tremor was amplified 100× with A-M Systems (Model 1700) and digitized with CED-micro 1401 at a sampling rate of 512 Hz. The results were saved in smr format for offline analysis using Spike-2 software (version 7.07). As shown in FIG. 9, a dose dependent inhibition of tremor was observed.

Immediately after completion of the tremor recording, mice were transferred to a designated necropsy room where they were anesthetized with CO₂. Blood was collected via the left ventricle at about 60 minutes post-harmaline, and brain tissue was collected and stored on dry ice.

Piezoelectric plate signal analysis: For the power spectral density analysis, electrical tremor signal was processed with fast Fourier transform (FFT) at a resolution of 0.5 Hz bins. PZ power density was computed from 20 minutes before and 20 minutes after the harmaline injection in a frequency range of 1 to 40 Hz. Non-normalized power density after harmaline administration was plotted in 0.5 Hz bins. Changes of power density after harmaline administration were normalized to the average of the 10-minute baseline and the average of the 20-minute baseline of each rat in 0.5 Hz bins. The average power density over the 8-13 Hz band and in the 9-12 Hz band was expressed in percent change relative to the baseline value individually. The average maximum power density across the 8-13 Hz band in 0.5 Hz bins was also calculated.

For the power-time analysis, the electrical tremor signal was process with FFT in time bin of 1 minute through the frequency range of 8-13 Hz and 9-12 Hz. Non-normalized average power density over the 8-13 Hz band and the 9-12 Hz band from 20-minutes pre- to 20-minutes post-harmaline administration in 1 minute bins was plotted. Changes of power density after harmaline administration was normalized to the average of the 10-minute baseline and the average of the 20-minute baseline of each rat in a time bin of 1 minute. The area under the power change time curve (AUC) over the 20-minute period after harmaline administration was calculated for each rat.

Statistical analysis: Both one-way ANOVA and two-way ANOVA followed by Bonferroni post-hoc test were applied for absolute data and normalized data (e.g., vehicle/harmaline vs. propranolol/harmaline).

### Example 6—Coadministration of Compound 1 and Compound 2

Rat Harmaline Induced Tremor (rHIT): The rHIT model was used to assess the impact of the combination of Compound 1 and Compound 2 on reducing the symptoms of tremor. Male SD rats (SLAC, Shanghai, China) weighing about 250 g at the time of testing were housed in a 12:12 light-dark cycle room throughout the study, with the lights on from 5 a.m. to 5 p.m, and fed ad libitum.

Six study groups (n=15 per group) included:
(1) administration of 5 mL/kg of vehicle 1 (0.5% methyl cellulose, 0.1% Tween08 in water, p.o.), 60 minutes prior to harmaline, followed by administration of vehicle 2 (15% Captisol, 5 mL/kg, p.o.) 30 minutes prior to administration of harmaline, and finally administration of harmaline (30 mg/kg, 5 mL/kg, i.p.);
(2) administration of Compound 2 (3 mg/kg, 5 mL/kg, p.o.) 60 minutes prior to harmaline, followed by administration of vehicle 2 30 minutes prior to harmaline, and finally administration of harmaline (30 mg/kg, 5 mL/kg, i.p.);
(3) administration of vehicle 1 60 minutes prior to administration of harmaline, followed by administration of Compound 1 (3 mg/kg, 5 mL/kg, p.o.) 30 minutes prior to administration of harmaline, and finally administration of harmaline (30 mg/kg, 5 mL/kg, i.p.);
(4) administration of Compound 2 (0.3 mg/kg, 5 mL/kg, p.o.) 60 minutes prior to harmaline, followed by administration of Compound 1 (3 mg/kg, 5 mL/kg, p.o.) 30 minutes prior to administration of harmaline, and finally administration of harmaline (30 mg/kg, 5 mL/kg, i.p.);
(5) administration of Compound 2 (1 mg/kg, 5 mL/kg, p.o.) 60 minutes prior to harmaline, followed by administration of Compound 1 (3 mg/kg, 5 mL/kg, p.o.) 30 minutes prior to administration of harmaline, and finally administration of harmaline (30 mg/kg, 5 mL/kg, i.p.); and
(6) administration of Compound 2 (3 mg/kg, 5 mL/kg, p.o.) 60 minutes prior to harmaline, followed by administration of Compound 1 (3 mg/kg, 5 mL/kg, p.o.) 30 minutes prior to administration of harmaline, and finally administration of harmaline (30 mg/kg, 5 mL/kg, i.p.).

The rHIT methods described in Example 5 were used. Specifically, on the day of testing, each rat was allowed to acclimate for at least thirty minutes in the test room. Prior to testing, rats were placed into the testing chamber for a five-minute offset to ensure the baseline activity was within the accepted range (3000-10000 V). Rats were then kept in the testing chamber to allow for a 20-minute baseline recording (T=−20 to 0 minutes) before harmaline treatment. before dosing according to the six groups noted above. After the 20-minute baseline recording, rats were dosed with harmaline and the post-harmaline effect was measured for 20 minutes. The piezoelectric plate signal analysis was conducted as described above in Example 5.

Immediately after completion of the tremor recording, mice were transferred to a designated necropsy room where they were anesthetized with CO₂. Blood was collected via the left ventricle at about 60 minutes post-harmaline, and brain tissue was collected and stored on dry ice.

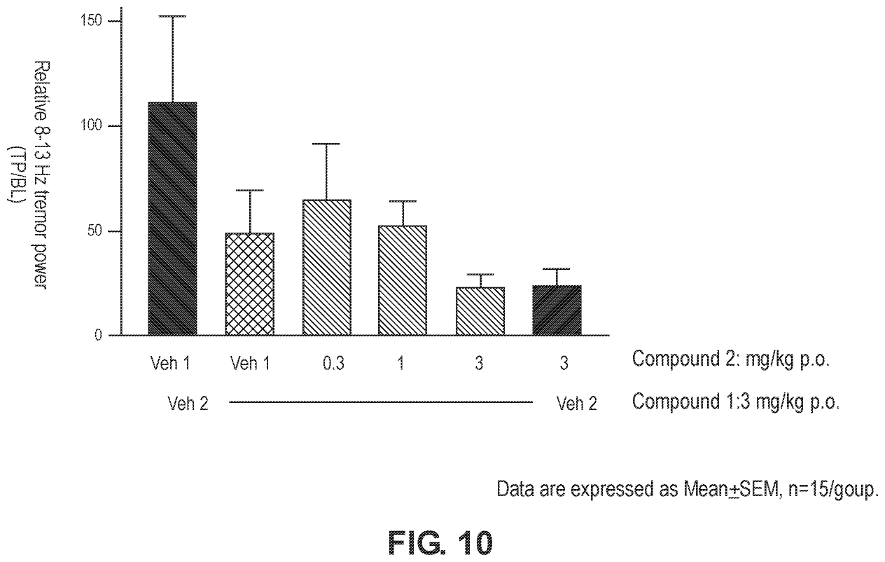

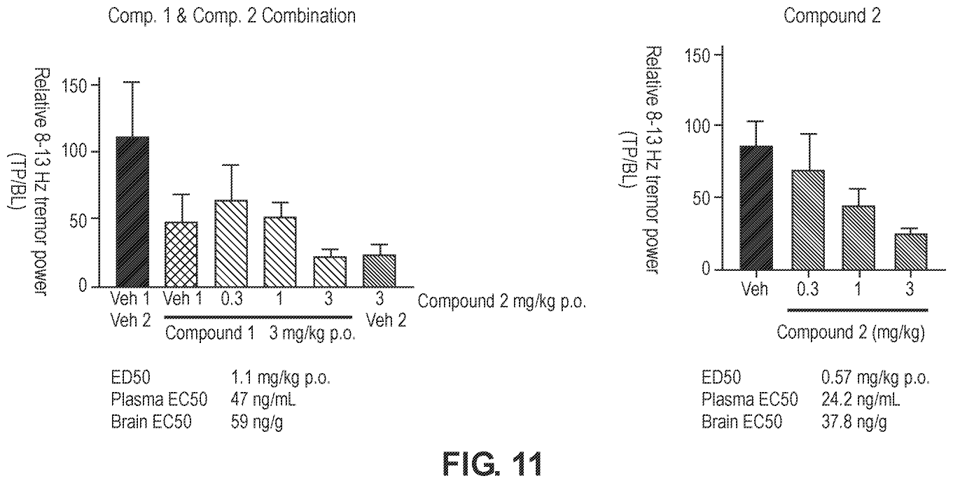

Results: A dose-dependent inhibition of tremor was observed (FIG. 10). While the addition of Compound 1 did not appear to enhance the tremor-reducing effects of Compound 2 (FIG. 11), because Compound 1 does not inhibit the efficacy of Compound 2 in treating tremor, it may be useful in treating comorbid symptoms such as anxiety and depression when used in combination with Compound 2 for treating tremor.

Spontaneous Locomotion Activity Test: Additionally, the effect of co-administration of Compound 1 and Compound 2 on spontaneous locomotion activity in male SD rats was evaluated. Male SD rats (SLAC, Shanghai, China) weighing about 250-300 g at the time of testing were housed in a 12:12 light-dark cycle room throughout the study, with the lights on from 5 a.m. to 5 p.m.

Formulations were prepared by making a stock solution of the highest dose and diluting down for the lower doses. For Compound 2, 0.5% methyl cellulose/0.1% Tween80 in water was added to Compound 2 to reach 0.6 mg/ml, and the vial was vortexed for 3 minutes and then sonicated in a water bath at room temperature for at least 30 minutes to obtain a clear solution. For Compound 1, 15% Captisol was added to Compound 1 to reach 0.6 mg/ml, and the mixture stirred at room temperature for 5 minutes. The solution was then vortexed for 3 minutes and sonicated for 45 minutes in a water bath at room temperature until a clear solution was obtained. All solutions were protected from light, and made fresh on each day of testing.

Four study groups (n=10 per group) included:
(1) administration of vehicle 1 (0.5% methyl cellulose/0.1% Tween80 in water, 5 mL/kg, p.o.) 60 minutes before testing, followed by administration of vehicle 2 (15% Captisol, 5 mL/kg, p.o.) 30 minutes before testing;
(2) administration of vehicle 1 60 minutes before testing, followed by administration of Compound 1 (3 mg/kg, 5 mL/kg, p.o.) 30 minutes before testing;
(3) administration of Compound 2 (3 mg/kg, 5 mL/kg, p.o.) 60 minutes before testing, followed by administration of vehicle 2 30 minutes before testing; and
(4) administration of Compound 2 (3 mg/kg, 5 mL/kg, p.o.) 60 minutes before testing, followed by administration of Compound 1 (3 mg/kg, 5 mL/kg, p.o.) 30 minutes before testing.

Dosing and behavioral observations were conducted by blinded observers. For the spontaneous locomotion test, naïve animals were acclimated in the test room for at least 30 minutes before dosing. Animals were dosed as indicated above at a volume of 5 mL/kg at the designated times. Spontaneous locomotion activity (sLMA) including travelling distance was measured for 30 minutes with a 1 minute sampling window (automatic tracking with video record in an isolated chamber). Results were reported in 5 minute bins. The light intensity was set at 45±5 Lux on the floor of the arena, and the plexiglass arena had a dimension of 40 cm×40 cm×30 cm.

For data analysis, a two-way ANOVA followed by Dunnett's post hoc test was used to detect significant differences in traveling distance in 5 minute bins between the vehicle and Compound 1 and Compound 2 groups. Additionally, ANOVA followed by Dunnett's test was used to detect significant differences in the total traveling distance of 30 minutes between the vehicle and Compound 1 and Compound 2 groups. All statistical analysis was conducted using GraphPadPrism 8.0.

Plasma and brain tissue samples were collected immediately after completion of the sLMA measurement, i.e., at the 90 minute time point for Compound 2 and the 60 minute time point for Compound 1.

### Example 7

Patients with essential tremor aged 18 to 65 years will be treated with an oral suspension of Compound 1 to study the safety, tolerability, pharmacokinetics, and efficacy of Compound 1 in the treatment of essential tremor (ET).

This clinical trial contains 2 parts. Part A is a randomized, double-blind, placebo-controlled, three-period, three-sequence, crossover design where participants will receive a single dose of 10 mg Compound 1, 20 mg Compound 2, or a matching placebo. Each participant will receive one dose of each treatment separated by a minimum 3-day washout between treatments. The sequence of treatment administration will be determined via randomization. Part A will consist of 3 study periods: Screening (up to 28 days), Intervention (approximately 9 days), and Safety Follow-up (approximately 3 days).

Part B is an open-label design where participants from Part A, after washout and confirmation of eligibility, may elect to participate in Part B. Determination of whether to proceed with Part B will be based on review of safety data from Part A. In Part B, all participants will receive 10 mg once every morning (QAM) for the first 14 days. Based on investigator judgment, the dose for Days 15 to 28 could be increased to 20 mg QAM. The decision to escalate to 20 mg or stay at 10 mg will be made by the Investigator on Day 14 based on safety and tolerability. Part B will consist of 3 study periods: Screening (up to 28 days, with a Part B screening visit only needed if the time between Day 1 of Part B and Visit 4 in Part A exceeds 28 days), Intervention (28 days), and Safety Follow-up (7 days). Confirmation of eligibility will occur prior to dosing on Day 1 of Part B.

Screening period: For Part A, the screening period will be up to 28 days. Prior to any procedures, participants will provide written informed consent. Key screening assessments include demographics, medical and disease history, prior and concomitant medications, physical examination, drug/alcohol screen, clinical laboratory evaluations, 12-lead ECG, vital signs, and assessments of ET severity using TETRAS Performance Subscale (TETRAS PS).

Following confirmation of eligibility, including review by the Eligibility Review Committee (ERC), participants will return to the clinic for the Baseline (Visit 2) assessments and for confirmation of eligibility. The ERC will integrate central video rating of the screening TETRAS PS with the past medical history of the participant and other screening assessments to determine eligibility of each participant for enrollment.

For Part B, participants who participate in Part A may continue to Part B, after providing written informed consent and following confirmation of study eligibility and completing selected screening assessments. During the screening period, participants will be asked to wear a CentrePoint Insight watch (ActiGraph Corp.) continuously for at least 7 Days prior to Day −1 and for the duration of the study (except for short intervals when the device is charging). This device will enable the continuous measurement of movement functions, general activity levels, and sleep patterns.

Intervention period: For Part A, participants who continue to meet all clinical trial entry criteria at Baseline (Visit 2) will be randomized in a 1:1:1 ratio to receive 1 of the 3 treatment sequences shown in Table 10 below.

TABLE 10
Treatment assignment by sequence

| | Period 1 | Period 2 | Period 3 |
|---|---|---|---|
| Sequence 1 | 10 mg | 20 mg | Placebo |
| Sequence 2 | 20 mg | Placebo | 10 mg |
| Sequence 3 | Placebo | 10 mg | 20 mg |

All study drug will be administered in the morning without food. Participants will refrain from consuming water from 1-hour pre-dose until 2 hours post-dose, excluding the amount of water consumed at dosing (240 mL). Participants may consume water ad libitum at all other times during the study. Food is allowed from 4 hours post-dose. On each dosing day, participants will take study drug in the clinic after pre-dose assessments and samples have been collected. There will be a 3-day (+3 days) washout between each dose of study drug. On each dosing day, efficacy and safety assessments will be performed. Efficacy assessments at these visits will include TETRAS PS or combined upper limb (CUL) assessment, which is comprised of items 4, 6, 7, and 8 from the TETRAS PS.

For Part B, participants who completed Part A and continue to meet all clinical trial entry criteria on Day 1 of Part B will be enrolled to receive 10 mg Compound 1 every morning for 14 days (Days 1 to 14) and then either: (1) escalate to 20 mg Compound 1 every morning for an additional 14 days (Days 15 to 28), or (2) stay at 10 mg Compound 1 every morning for an additional 14 days (Days 15 to 28). All study drug will be administered every morning (on an empty stomach).

On Visit 2 and Visit 4, efficacy and safety assessments will be performed, including a selection of the following endpoints: the TETRAS Activities of Daily Living (TETRAS ADL), CGI-S, CGI-I, Patient Global Impression of Change (PGI-C), BDI-II, and BAI. On Day 28, participants will return to the clinic for safety and efficacy assessments.

Participants will continue to wear the CentrePoint Insight watch (ActiGraph Corp.) for the duration of the Intervention Period. The built-in inertial sensors in the wearable device will passively measure their movement functions, general activity levels, and sleep patterns, and should be worn continuously day and night, except for brief intervals when the device is charging. Participants will be asked to record the time they took study drug each morning by pressing a button on the wearable device.

Safety follow-up period: For Part A, the safety follow-up period will be approximately 3 days (+2 days).

For Part B, the safety follow-up period will be approximately 7 days (±2 days).

Safety monitoring: For both Part A and Part B, safety will be monitored throughout the duration of the clinical trial by the evaluation of physical examination, SSS (Part A only), clinical laboratory evaluations (chemistry, hematology, and urinalysis), vital signs, 12-lead ECGs, and C-SSRS. AEs and concomitant medications will be monitored from the time of consent to the end of clinical trial participation.

Inclusion criteria: For both Part A and Part B, a participant should meet all the following criteria (at Screening unless otherwise specified) to be eligible to participate in this clinical trial:
Willing and able to understand and sign an informed consent document indicating that he/she understands the purpose of the clinical trial, understands the procedures that are required for the clinical trial, and is willing to participate in the clinical trial and comply with the protocol;
Is male or female aged at least 18 years of age;
Has a body mass index (BMI) between 18 and 40 kg/m², inclusive;
Has a clinical diagnosis of moderate to severe ET, including: (a) tremor syndrome of bilateral upper limb action tremor, (b) symptoms for at least 3 years in duration, (c) with or without tremor in other locations (e.g., head, voice, or lower limbs), and (d) if the symptoms and signs are judged by the investigator to be due to the diagnosis of ET, it is acceptable for them to also have 1 or more of the following ET plus signs: (i) mild dystonic posturing, (ii) mild rest tremor in the setting of advanced ET and in the absence of other features of Parkinsonism; (iii) intention tremor, and (iv) mild increase in tandem gait difficulty.
Has a TETRAS upper limb score (i.e., sum of bilateral upper limb items 4a, 4b, and 4c) of ≥10 as rated by the Investigator at Screening and Baseline. Note that for Part B, tremor severity can fluctuate, and it may be that a participant who met TETRAS upper limb criteria at Screening and Day 1 for Part A no longer meets the tremor severity inclusion criterion above at Screening or on Day 1 of Part B. If all the other eligibility criteria are still met such a participant would still be eligible for inclusion in Part B;
If receiving primidone or topiramate for ET, is willing and able to complete discontinuation no later than 14 days prior to Day 1 of Part A. If currently receiving any other medication for ET, is on a stable dose of any of these other medications for ET for 28 days prior to Screening and is willing to maintain stable doses throughout the clinical trial;
Is willing to use medically acceptable method of contraception; and
Has been assessed as an appropriate and suitable candidate by the ERC, Medical Monitor and Sponsor including central video review confirmation of the screening TETRAS UL score prior to Baseline.

Exclusion criteria: For both Part A and Part B, a participant who meets any of the following criteria (at Screening unless otherwise specified) will be excluded from this clinical trial:
Has a history or clinical evidence of other medical, neurological, or psychiatric condition that may explain or cause tremor, including but not limited to Parkinson's disease, Huntington's disease, Alzheimer's disease, cerebellar disease (including spinocerebellar ataxias), primary dystonia, Fragile X Tremor/Ataxia syndrome or family history of Fragile X syndrome, traumatic brain injury, psychogenic tremor, alcohol or benzodiazepine abuse or withdrawal, multiple sclerosis, polyneuropathy, and endocrine states such as hyperthyroidism or unstable treatment of hypothyroidism or medication, food, or supplement induced movement disorders (e.g., tremor related to beta agonists or caffeine), or other medical, neurological, or psychiatric conditions that may explain or cause tremor;
Has a lifetime history of seizures, including febrile seizures;
Has trauma to the nervous system within 3 months preceding the onset of tremor;
Has had prior magnetic resonance-guided focused ultrasound or surgical intervention for ET such as deep brain stimulation or thalamotomy;
Has had botulinum toxin injection for ET in the 6 months prior to Screening;
Is using the Cala trio health device for ET in the last 14 days prior to Baseline and throughout the study;
Is unwilling or unable to refrain from episodic use of medication(s)/substance(s) that might interfere with the evaluation of tremor during the trial. Stable use of medication(s)/substance(s) that might impact tremor, including caffeine and beta-agonist bronchodilators, is allowed so long as the tremor is judged by the Investigator to be primarily due to the participant's ET diagnosis.
Is unwilling or unable to refrain from use of any sleep aids (e.g., eszopiclone, zaleplon, and zolpidem) or anxiolytics that are known to be mediated by GABAergic mechanisms (e.g., benzodiazepines) within 24 hours prior to any clinic visit. Exception: Stable use (at least 28 days prior to Screening) of up to 2 ET non-tremor active, non-GABAergic antidepressants and anxiolytics is allowed during the clinical trial after discussion with the medical monitor and/or sponsor designee. Note for Part B only: Participants should avoid using sleep aids and anxiolytics that are known to be mediated by GABAergic mechanisms.
Is required to take any excluded medication of the protocol during the clinical trial. Chronic medication must be stable for at least 4 weeks prior to Screening. Any new non-urgent medications started during the trial and permitted should be discussed with the Sponsor before initiation, as practicable;
Has received any experimental or investigational drug, device, or other therapy within 30 days or 5 half-lives (whichever is longer) prior to Screening;
Has a known hypersensitivity to any component of the formulation of Compound 1;
Is unwilling or unable to refrain from alcohol 24 hours before and during clinical trial visits, or regular consumption of more than 2 standard alcohol-containing beverages per day for males or more than 1 standard alcohol-containing beverages per day for females and are unwilling to reduce consumption to the appropriate level during the Screening period and maintain this level throughout the Intervention and Follow-up Periods;
Has a history of substance use disorder consistent with DSM-5 criteria in the opinion of the Investigator;
Is at risk for suicide according to the Investigator's clinical judgment, such as active suicidal thoughts with intent or plan, or has made a suicide attempt in the past 12 months prior to Screening.
Is currently pregnant or breastfeeding or is planning to become pregnant during the clinical trial or within 3 days of the last study drug dose;
Is currently diagnosed with cancer, being treated for cancer, or has a history of cancer treatment within the last 5 years prior to Screening, Exception: Adequately treated basal cell carcinoma, adequately treated stage I squamous cell skin cancer, or adequately treated in situ cervical cancer within the 5 years prior to screening;
Has any of the following abnormal test result at Screening: a serum total bilirubin value >1.5×upper limit of normal (ULN); a serum alanine aminotransferase (ALT) or aspartate aminotransferase (AST) value >2×ULN. As an exception, participants that present with elevated bilirubin in the absence of elevations in ALT or AST that fits the pattern of Gilbert's syndrome may be enrolled after discussion with the medical monitor and/or sponsor designee if their conjugated bilirubin is below the ULN;
Has a QT interval with Fridericia correction method (QTcF) >450 msec, confirmed with 1 repeat testing as needed, at Screening or Baseline, or the participant has long QT syndrome or is being treated with Class 1A (e.g., quinidine, procainamide) or Class 3 (e.g., amiodarone, sotalol) antiarrhythmic drugs; or any abnormal ECG findings confirmed as clinically significant by the Investigator in consultation with the medical monitor and/or sponsor designee;
Has any other significant disease, disorder, laboratory abnormalities, or environmental factor that, in the opinion of the Investigator or sponsor designee, may either put the participant at risk due to participation in the clinical trial, may influence or confound the result of the clinical trial, or affect the participant's ability to participate in the clinical trial;
Has a pacemaker, implantable cardioverter-defibrillator or any other implanted stimulation device including a deep brain stimulator (DBS) stimulator;
Has a history of active (clinically significant) skin disorders;
Has a history of allergic response to silicones or adhesives; or
Has broken, damaged, or irritated skin or rashes near the sensor application sites.

All participants will receive Compound 1 or matching placebo (collectively referred to as study drug) in an oral tablet formulation. All study drug will be administered in fasting state for single doses and every morning for multiple day dosing.

Sample size: Approximately 15 participants will be randomized to 1 of 3 possible treatment sequences in a 1:1:1 manner to achieve approximately 12 evaluable participants in Part A.

Statistical methods: For all data displays, separate summaries will be generated for each part of the clinical trial. Safety, tolerability, PK, and efficacy variables will be summarized using descriptive statistics. Descriptive summaries for categorical variables will include counts and percentage. Descriptive summaries for continuous variables will include number of participants (n), mean, standard deviation (SD), median, minimum, and maximum. Summaries will be presented by timepoint, where appropriate.

Efficacy analyses: For Part A, reported values and change from pre-dose values in TETRAS CUL score will be summarized descriptively by part, treatment, and timepoint. The change from pre-dose for the TETRAS CUL score will be analyzed with mixed model repeated measures (MMRM) methods using the full analysis set. The model will include treatment, period, sequence, timepoint, and treatment-by-timepoint interaction as fixed effects and period-specific pre-treatment baseline as a covariate. The Kenward-Roger approximation (Kenward and Roger 1997) will be used to estimate denominator degrees of freedom. The comparison of interest will be the difference between each Compound 1 dose level and placebo conditions at each timepoint. Model based point estimates (i.e., least square [LS] means for each treatment condition and the treatment difference), 95% confidence intervals (CIs) for the difference, and p-value will be reported for each timepoint. Additional MMRM models may be used with pre-dose baseline and post-dose reported values included as response variables.

For Part B, reported values and change from baseline values in TETRAS ADL will be summarized descriptively by timepoint.

Safety analyses: Adverse event data will be coded using the Medical Dictionary for Regulatory Activities (MedDRA). The number and percentage of participants experiencing any treatment-emergent adverse event (TEAE) will be summarized by part, treatment, system organ class, and preferred term. SAEs and TEAEs leading to discontinuation of study drug will be summarized similarly. In addition, TEAEs will be summarized by maximum severity and relationship to study drug.

Reported values and change from pre-dose values in vital signs, ECG parameters, and SSS score will be summarized by part, treatment, and timepoint, where appropriate. Reported values and change from Screening values in hematology and clinical chemistry laboratory parameters will be listed. For the C-SSRS, the incidence of suicidal ideation/behavior at any time will be summarized by part and treatment for each C-SSRS ideation, behavior, and ideation/behavior indicator.

Pharmacokinetic analyses: For Part A, plasma concentrations of Compound 1 and its metabolites will be summarized by treatment and nominal sampling timepoint. For Part B, plasma concentrations of Compound 1 and its metabolites will be summarized by nominal sampling timepoint. Descriptive statistics for plasma concentrations will include n, number of participants with concentrations below the limit of quantification (BLQ), mean, SD, coefficient of variation (CV %), median, minimum, and maximum. For descriptive summaries, plasma concentrations reported as BLQ will be set to zero.

For Part A, PK parameters will be calculated using noncompartmental methods and summarized using descriptive statistics. Collected plasma concentration data may also contribute to population PK analyses.

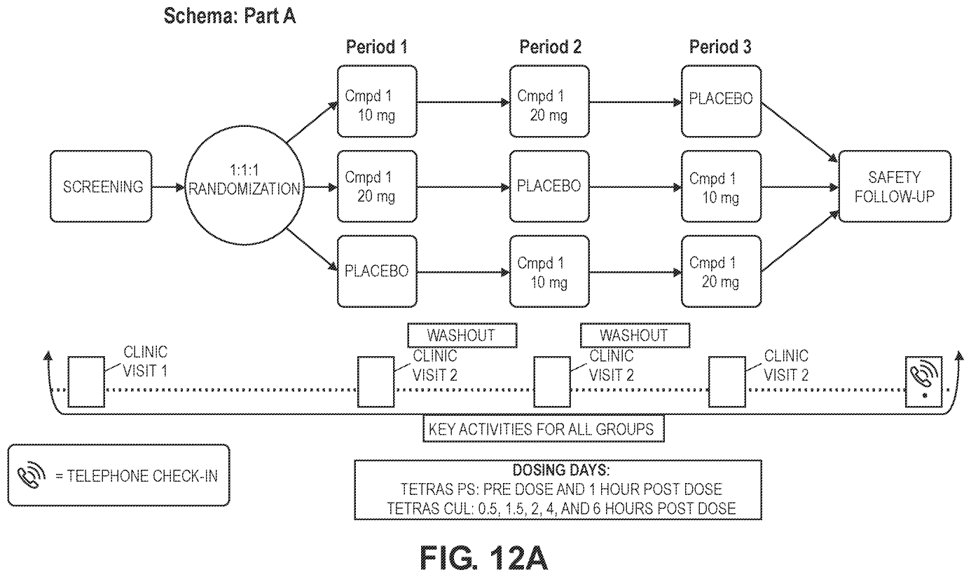

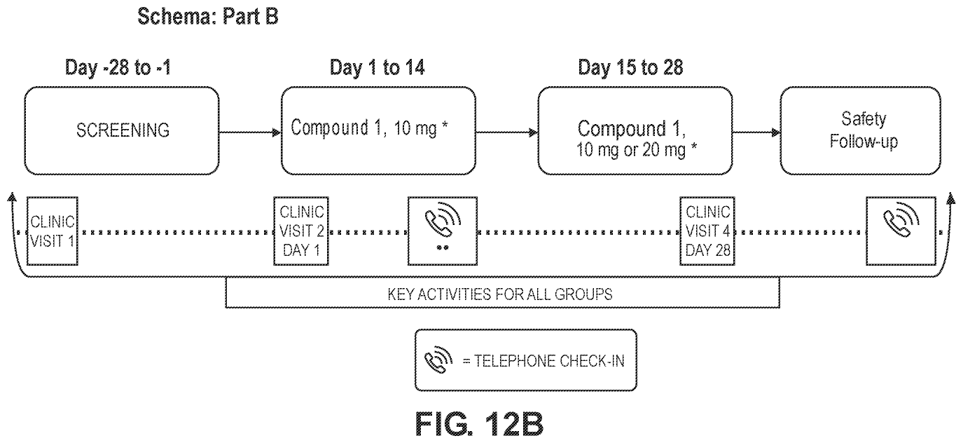

The schema for Part A and Part B of the protocol are illustrated in FIG. 12A and FIG. 12B, respectively.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as acknowledgment or any form of suggestion that they constitute valid prior art or form part of the common general knowledge in any country in the world.

What is claimed is:

1. A method of treating major depressive disorder (MDD), motor disorders, and/or musculoskeletal conditions in a patient in need thereof comprising orally administering a therapeutically effective amount of Compound 1: 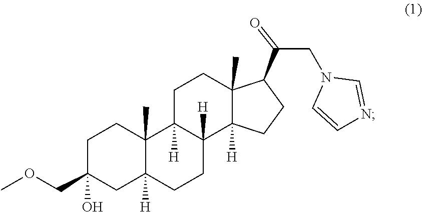
or a pharmaceutically acceptable salt thereof to a patient in need thereof, in the evening.

2. The method of claim 1, wherein the evening is about two hours after the evening meal.

3. The method of claim 1, wherein the evening is about four hours after the evening meal.

4. The method of any one of claims 1-3, wherein the evening is between about 5 p.m. and about 12 a.m.

5. The method of any one of claims 1-4, wherein the evening is about two hours prior to bedtime.

6. A method of reducing at least one side effect associated with administering a therapeutically effective amount of Compound 1: 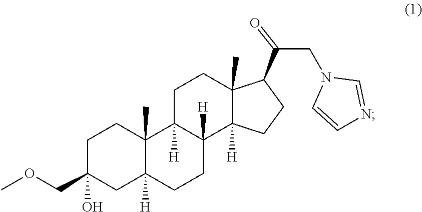
or a pharmaceutically acceptable salt thereof to a patient in need thereof, comprising administering Compound 1 in the evening, wherein the at least one side effect reduced is chosen from somnolence, sedation, dizziness, lightheadedness, fatigue, euphoria, feeling drunk, feeling abnormal, headache, cognitive disorder, disturbance of attention, memory impairment, slowed cognition, decreased alertness, constipation, or diarrhea.

7. The method of claim 6, wherein the at least one side effect associated with the administering of Compound 1 or a pharmaceutically acceptable salt are determined by the guidelines set forth in Guidance for Industry and Investigators: Safety Reporting Requirements for INDs and BA/BE Studies (2012), described in the ‘NIH-FDA Phase 2 and 3 IND/IDE Clinical Trial Protocol Template’, and reported with standardized terms according to the Medical Dictionary for Regulatory Activities.

8. A method of treating a motor disorder in a patient in need thereof comprising orally administering a therapeutically effective amount of Compound 1: 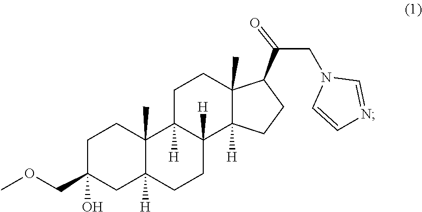
or a pharmaceutically acceptable salt thereof to a patient in need thereof.

9. The method of claim 8, wherein said administering is in the evening.

10. The method of claim 9, wherein the evening is about two hours after the evening meal.

11. The method of claim 9, wherein the evening is about four hours after the evening meal.

12. The method of any one of claims 9-11, wherein the evening is between about 5 p.m. and about 12 a.m.

13. The method of any one of claims 9-12, wherein the evening is about two hours prior to bedtime.

14. The method of claim 8, wherein said administering is during the day.

15. The method of any of claims 8-14, wherein the motor disorder is chosen from cerebellar tremor, intention tremor, dystonic tremor, essential tremor, orthostatic tremor, Parkinsonian tremor, physiological tremor, psychogenic tremor, or rubral tremor.

16. The method of any of claims 8-15, further comprising co-administering Compound 2: 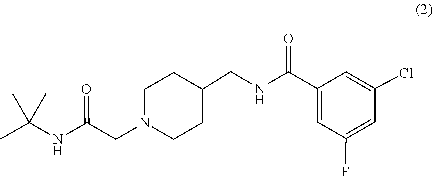 [SMILES: CC(C)(C)NC(=O)CN1CCC(CNC(=O)c2cc(F)cc(Cl)c2)CC1]
or a pharmaceutically acceptable salt thereof.

17. A method of treating a musculoskeletal condition in a patient in need thereof comprising orally administering a therapeutically effective amount of Compound 1: 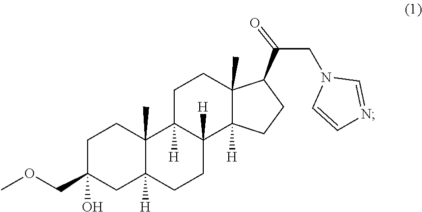
or a pharmaceutically acceptable salt thereof to a patient in need thereof.

18. The method of claim 17, wherein said administering is in the evening.

19. The method of claim 18, wherein the evening is about two hours after the evening meal.

20. The method of claim 18, wherein the evening is about four hours after the evening meal.

21. The method of any one of claims 18-20, wherein the evening is between about 5 p.m. and about 12 a.m.

22. The method of any one of claims 18-21, wherein the evening is about two hours prior to bedtime.

23. The method of claim 17, wherein said administering is during the day.

24. The method of any of claims 17-23, wherein the musculoskeletal condition is chosen from fibromyalgia, polymyalgia, chronic fatigue syndrome, or systemic exertion intolerance disease.

25. The method of claim 24, wherein the musculoskeletal condition is fibromyalgia.

26. A method of treating essential tremor in a patient in need thereof comprising orally administering a therapeutically effective amount of Compound 1: 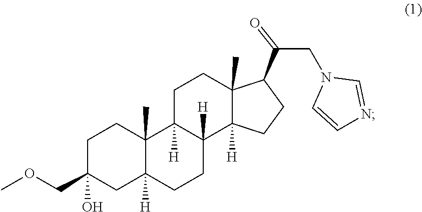
or a pharmaceutically acceptable salt thereof to a patient in need thereof.

27. The method of claim 26, wherein said administering is in the evening.

28. The method of claim 27, wherein the evening is about two hours after the evening meal.

29. The method of claim 27, wherein the evening is about four hours after the evening meal.

30. The method of any one of claims 27-29, wherein the evening is between about 5 p.m. and about 12 a.m.

31. The method of any one of claims 27-30, wherein the evening is about two hours prior to bedtime.

32. The method of claim 26, wherein said administering is during the day.

33. The method of any of claims 26-32, further comprising co-administering Compound 2: 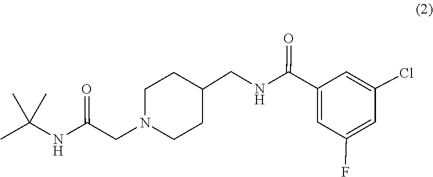 [SMILES: CC(C)(C)NC(=O)CN1CCC(CNC(=O)c2cc(F)cc(Cl)c2)CC1]
or a pharmaceutically acceptable salt thereof.
